(19) United States
(12) Patent Application Publication
(10) Pub. No.: US 2008/0275117 A1
(43) Pub. Date: Nov. 6, 2008

(54) Compositions and Methods Comprising Boswellia Species

(76) Inventors: Dan Li, Singapore (SG); Robert T. Gow, Naples, FL (US); George W. Sypert, Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/859,528

(22) Filed: Sep. 21, 2007

Related U.S. Application Data
(60) Provisional application No. 60/846,204, filed on Sep. 21, 2006.

(51) Int. Cl. A61K 31/22; A61P 29/00; A61P 19/02; C07H 1/08; A61K 31/19

(52) U.S. Cl. 514/546; 514/572; 536/128

(57) ABSTRACT

In certain aspects the invention features novel compositions and pharmaceutical preparations of the same. In certain embodiments, the compositions comprise α- and/or β-boswellic acid and/or their C-acetates in an amount greater than 65% by weight.

### RELATED APPLICATIONS

This application incorporates by reference, and claims the benefit of priority to U.S. Provisional Application No. 60/846,204, filed on Sep. 21, 2006.

### FIELD OF INVENTION

The present invention relates to compositions and methods for making compositions derived from Boswellia species (frankincense or olibanum) having uniquely elevated volatile oil, boswellic acids, and polysaccharide compounds, particularly, human oral delivery formulations, and methods for use of such compositions.

### BACKGROUND OF THE INVENTION

The oleo-gum-resin exudate of Boswellia species (Family Burseraceae, tribe Busereae, subtribe Boswellinae), also termed frankincense or olibanum, has been used for thousands of years in Ayurvedic medicine of India for the treatment of inflammatory diseases, including arthritis, bronchitis, colitis, and low back pain (1,2). The four main gum-resin producing species include B. carteri (East Africa), B. frerena (Somalia), B. sacra (Southern Arabia), and B. serrata (North-Western India). The oleo-gum-resins from India (B. serrata) and Africa (B. carteri) are the most commonly used for phytopharmaceutical or medicinal preparations (3).

### SUMMARY OF INVENTION

In certain aspects the invention features novel compositions and pharmaceutical preparations of the same. In certain embodiments, the compositions comprise α- and/or β-boswellic acid and/or their C-acetates in an amount greater than 65% by weight.

In one embodiment, the α- and β-boswellic acids or their C-acetates comprise alpha-boswellic acid (α-BA), acetyl-alpha-boswellic acid (α-ABA), beta-boswellic acid (β-BA), acetyl-beta-boswellic acid (β-ABA), 9.11-dehydro-alpha-boswellic acid (D-α-BA), acetyl-9.11-dehydro-alpha-boswellic acid (D-α-ABA), 9.11-dehydro-beta-boswellic acid (D-β-BA), acetyl-9.11-dehydro-beta-boswellic acid (D-β-ABA), lupeolic acid (LA), acetyl-lupeolic acid (ALA), 11-keto-beta-boswellic acid (β-KBA), and/or acetyl-11-keto-beta-boswellic acid (β-AKBA).

Further embodiments feature compositions comprising n-octyl acetate, incensole, incensole acetate, linalol, borneol, camphene, elemene, caryophyllene, incensole oxide, incensole oxide acetate, or combinations thereof in an amount of at least about 5, 10, 15, 20, 25, or 30% by weight.

Further embodiments feature compositions comprising a polysaccharide. In a further embodiment, the polysaccharide comprises dextran, glucose, arabinose, galactose, rhamnose, xylose or uronic acid in an amount of at least about 5, 10, 15, 20, 25, 30, or 35% by weight.

Further embodiments feature compositions comprising 1,3-di-t-butylbenzene, 1-undecanol, dodecanoic acid, 4-tetradecanol, viridiflorol, nerodidol isobutyrate, octadecanoic acid, butyl acetate, dimer of alpha-phellandrene, alpha-amyrenone, beta-amyrin, 3-epi-alpha-amyrin, 3-epi-beta-amyrin, or lupeenone.

Further embodiments feature compositions comprising combinations of the above compositions.

Further embodiments feature any of the aforementioned compositions wherein the amount of α- and/or β-boswellic acid and/or their C-acetates is greater than about 70, 75, 80, 85, 90, or 95% by weight.

In another aspect, the present invention features a composition comprising 30-70% by weight of α- and/or β-boswellic acid and/or their C-acetates; 5-25% by weight n-octyl acetate, incensole, incensole acetate, linalol, borneol, camphene, elemene, caryophyllene, incensole oxide, incensole oxide acetate, or combinations thereof; and 5-50% by weight polysaccharide.

In a further embodiment, the amount of α- and/or β-boswellic acid and/or their C-acetates is 40-60% by weight; the amount of n-octyl acetate, incensole, incensole acetate, linalol, borneol, camphene, elemene, caryophyllene, incensole oxide, incensole oxide acetate, or combinations thereof is 10-20% by weight; and the amount of polysaccharide is 30-40% by weight.

In a further embodiment, the α- and β-boswellic acids and/or their C-acetates comprise α-BA, β-ABA, β-BA, β-ABA, D-α-BA, D-α-ABA, D-β-BA, D-β-ABA, LA, ALA, β-KBA, or β-AKBA. In a further embodiment, the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

In a further embodiment, the polysaccharide comprises dextran, glucose, arabinose, galactose, rhamnose, xylose or uronic acid. In a further embodiment, the polysaccharide comprises dextran, glucose, arabinose, galactose, rhamnose, xylose and uronic acid.

In another aspect, the present invention features a method for making a Boswellia species extract having at least one predetermined characteristic comprising: sequentially extracting a Boswellia species plant material to yield an essential oil fraction, a boswellic acid or acetylated boswellic acid fraction, and a polysaccharide fraction, wherein the lipid soluble chemical constituents are derived by extracting plant feedstock material by solvent extraction; the essential oil fraction or sub-fraction is extracted from the lipid soluble extraction by supercritical chromatography extraction; the boswellic acid fraction is the remainder of the supercritical chromatography extraction process and may be acetylated to produce an acetylated boswellica acid fraction; and the polysaccharide fraction is derived by hot water extraction of the remainder of the lipid soluble chemical constituent extraction.

In a further embodiment, the method for essential oil extraction comprises: a) loading in an extraction vessel, an affinity adsorbent and a lipid soluble chemical constituent extract mixture; b) adding carbon dioxide under supercritical conditions; c) contacting the feedstock-affinity adsorbent mixture and the carbon dioxide for a time; and d) collecting an essential oil fraction in a collection vessel.

In a further embodiment, a supercritical chromatography carbon dioxide fractional separation system is used for fractionation, purification, and profiling (altering the essential oil chemical constituent compound ratios) of the essential oil fraction.

In a further embodiment, the method for boswellic acid fraction extraction comprises optimizing the supercritical chromatography extraction conditions to produce a highly concentrated boswellic acid (triterpenoid) residue extract.

In a further embodiment, the aforementioned method comprises the additional step of acetylating the purified boswellic acid fraction to produce a highly concentrated acetylated boswellic acid composition wherein about 90% of the non-acetylated boswellic acids have been converted into their acetylated forms.

In a further embodiment, the method for polysaccharide fraction extraction comprises: a) contacting a remainder of a feedstock material from a lipid soluble chemical constituent solvent extraction or ground Boswellia oleo-gum-resin material with a hot water solution for a time sufficient to extract polysaccharide chemical constituent; and b) separating and purifying the solid polysaccharides from the solution by ethanol precipitation.

In another aspect, the present invention features any of the aforementioned compositions further comprising a pharmaceutical carrier. The compositions of the present invention may comprise pastes, resins, oils, beverage, liquid infusion or decoction, powders, and dry flowable powders. Such products are processed for many different uses, including, but not limited to, a fast dissolve tablet or other oral delivery form. The Boswellia species compositions taught herein can be formulated alone or in combinations with other compounds such as other botanical materials, herbal remedies, pharmaceutical agents, food, dietary supplement, or beverages. In a further embodiment, the pharmaceutical composition further comprises boswellia constituents in combination with a synergistic amount of curcumin. The Boswellia species compositions can be used for treatment and prevention of physiological, psychological and medical conditions.

The compositions of the present invention are useful in providing the physiological, psychological, and medical effects including, but not limited to, anti-inflammatory, anti-osteoarthritis, anti-rheumatoid arthritis, low back pain, anti-spasmodic, analgesia, anti-bronchitis, anti-bronchial asthma, prevention and treatment of chronic colitis, ulcerative colitis, ileitis, and Crohn's disease, cancer prevention and treatment, immune enhancement, anti-oxidant activity, atherosclerotic cardiovascular disease and stroke prevention and therapy, and anti-viral and anti-herpes activity.

In another aspect, the present invention features a method of treating arthritis in a subject comprising administering to a subject in need thereof a therapeutically effective amount of the aforementioned pharmaceutical compositions.

In another aspect, the present invention features a boswellia species extract comprising a fraction having a Direct Analysis Real Time (DART) mass spectrometry chromatogram of any one of FIG. 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, or 92.

In a further embodiment, the extract comprises a compound selected from the group consisting of essential oils, boswellic acids, acetylated boswellic acids, and polysaccharides.

In a further embodiment, said compound is selected from the group consisting of the α- and β-boswellic acids and/or their C-acetates comprise alpha-boswellic acid (α-BA), acetyl-alpha-boswellic acid (α-ABA), beta-boswellic acid (β-BA), acetyl-beta-boswellic acid (β-ABA), 9.11-dehydro-alpha-boswellic acid (D-α-BA), acetyl-9.11-dehydro-alpha-boswellic acid (D-α-ABA), 9.11-dehydro-beta-boswellic acid (D-β-BA), acetyl-9.11-dehydro-beta-boswellic acid (D-β-ABA), lupeolic acid (LA), acetyl-lupeolic acid (ALA), 11-keto-beta-boswellic acid (β-KBA), acetyl-11-keto-beta-boswellic acid (β-AKBA), and combinations thereof.

In a further embodiment, the amount of boswellic acids or acetylated boswellic acids is greater than 65% by weight.

In a further embodiment, the amount of essential oil is from 65% to 90% by weight.

In a further embodiment, the polysaccharide is selected from the group consisting of glucose, arabinose, galactose, rhamnose, xylose uronic acid and combinations thereof. In a further embodiment, the amount of polysaccharide is greater than 15% by weight. In a further embodiment, the amount of polysaccharide is from 25% to 90% by weight. In a further embodiment, the amount of polysaccharide is from 50% to 90% by weight. In a further embodiment, the amount of polysaccharide is from 75% to 90% by weight.

In another aspect, the present invention features a food or medicament comprising any one of the aforementioned boswellia species extracts.

These embodiments of the present invention, other embodiments, and their features and characteristics, will be apparent from the description, drawings and claims that follow.

### BRIEF DESCRIPTION OF THE DRAWINGS

FIG. 1 depicts an exemplary method for the preparation of a concentrated extract solution from Boswellia feedstock.

FIG. 2 depicts an exemplary method for the preparation of a purified Boswellia fraction using a polymer adsorbent.

FIG. 3 depicts an exemplary method for the preparation of acetylated Boswellic acid fraction.

FIG. 4 depicts an exemplary method for the preparation of polysaccharide fractions.

FIG. 5 depicts the chemical structures of α- and β-boswellic acids and their C-acetates.

FIG. 6 depicts chemical constituents of Boswellia species.

FIG. 7 depicts major compounds identified in Boswellia oleo-gum-resin (7,8,11-11b, 14).

FIG. 8 (A-E) depicts selected major compounds identified in Boswellia essential oil.

FIG. 9 depicts selected chemical composition of Boswellia carteri.

FIG. 10 depicts selected physical properties of silica gel affinity adsorbents.

FIG. 11 depicts HPLC analysis results of boswellic acid reference standards at a concentration of 1.0 mg/mL in methanol.

FIG. 12 depicts colorimetric analysis of dextran standards.

FIG. 13 depicts boswellia Et-OH leaching extraction yield and boswellic acid yield.

FIG. 14 depicts extraction yields of each stage of processing in typical examples of multi-stage SCCEP silica gel extraction/fractionation at fixed temperatures of 40° C. and 80° C.

FIG. 15 depicts HPLC analysis results of boswellic acid purity of residue from SCCEP silica gel processing.

FIG. 16 depicts HPLC analytical results of boswellic acid purity of residue from SCCEP surface modified silica gel processing.

FIG. 17 depicts HPLC analytical results of acetylated purified boswellic acid fraction.

FIG. 18 depicts polysaccharide fraction extraction yield and purity. [Table 13P]

FIG. 19 depicts ingredients for formulations described in (A) Example 7 and (B) Example 8.

FIG. 20 depicts DART-MS of 80% ethanol extract (HS700) at room temperature.

FIG. 21 (A-B) depicts compounds from the proprietary chemical database that have been identified in 80% ethanol extract of B. carteri (HS700).

FIG. 22 depicts a DART-MS of the 100% ethanol (HS701) crude extract at room temperature.

FIG. 23 (A-B) depicts compounds from the proprietary chemical database that have been identified in 80% ethanol extract of B. carteri (HS701).

FIG. 24 depicts a DART-MS of the 40° C. distilled water crude extract (HS702).

FIG. 25 (A-B) depicts compounds from the proprietary chemical database that have been identified in the 40° C. distilled water crude extract (HS702).

FIG. 26 depicts a DART-MS of 20% ethanol (40° C.) extract (HS703).

FIG. 27 (A-B) depicts compounds from the proprietary chemical database that have been identified in the 20% ethanol (40° C.) extract (HS703).

FIG. 28 depicts a DART-MS of 40% ethanol crude extract at 40° C.

FIG. 29 (A-B) depicts compounds from the proprietary chemical database that have been identified in the 40% ethanol crude extract at 40° C. (HS704).

FIG. 30 depicts a DART-MS of the 60% ethanol crude extract at 40° C.

FIG. 31 (A-B) depicts compounds from the proprietary chemical database that have been identified in the 60% ethanol crude extract at 40° C. (HS705).

FIG. 32 depicts a DART-MS of the 80% ethanol extract at 40° C. (HS706).

FIG. 33 (A-B) depicts compounds from the proprietary chemical database that have been identified in the 80% ethanol extract at 40° C. (HS706).

FIG. 34 depicts a DART-MS of the 100% ethanol crude extract at 40° C.

FIG. 35 (A-B) depicts compounds from the proprietary chemical database that have been identified in 100% ethanol crude extract at 40° C. (HS707).

FIG. 36 depicts DART-MS of the 80% ethanol crude extract at 60° C.

FIG. 37 (A-B) depicts compounds from the proprietary chemical database that have been identified in the 80% ethanol crude extract at 60° C. (HS708).

FIG. 38 depicts a DART-MS of the 100% ethanol crude extract at 60° C. (HS709) from HS00005 feedstock.

FIG. 39 (A-B) depicts compounds from the proprietary chemical database that have been identified in the 100% ethanol crude extract at 60° C. (HS709).

FIG. 40 depicts a DART-MS of HS710; Bosswellia carteri extract HS700 after acetylation.

FIG. 41 (A-B) depicts compounds from the proprietary chemical database that have been identified in HS710.

FIG. 42 depicts a DART TOF-MS spectrum of Boswellia carteri polysaccharide precipitate.

FIG. 43 (A-B) depicts identified compounds in Boswellia carteri polysaccharide precipitate.

FIG. 44 depicts a DART TOF-MS spectrum of the S1 extract.

FIG. 45 (A-B) depicts identified compounds in the S1 extract.

FIG. 46 depicts a DART TOF-MS spectrum of the S2 extract.

FIG. 47 (A-B) depicts identified compounds in the S2 extract.

FIG. 48 depicts a DART TOF-MS spectrum of the S3 extract.

FIG. 49 (A-B) depicts identified compounds in the S3 extract.

FIG. 50 depicts a DART TOF-MS spectrum of the S4 extract.

FIG. 51 (A-B) depicts identified compounds in the S4 extract.

FIG. 52 depicts a DART TOF-MS spectrum of the S5 extract.

FIG. 53 (A-B) depicts identified compounds in the S5 extract.

FIG. 54 depicts a DART TOF-MS spectrum of the S6 extract.

FIG. 55 (A-B) depicts identified compounds in the S6 extract.

FIG. 56 depicts a DART TOF-MS spectrum of the S7 extract.

FIG. 57 (A-B) depicts identified compounds in the S7 extract.

FIG. 58 depicts a DART TOF-MS spectrum of a boswellia species extraction process using silica gel 633, stage 1.

FIG. 59 depicts identified compounds in the boswellia species extraction process using silica gel 633, stage 1.

FIG. 60 depicts a DART TOF-MS spectrum of a boswellia species extraction process using silica gel 633, stage 2.

FIG. 61 (A-B) depicts identified compounds in the boswellia species extraction process using silica gel 633, stage 2.

FIG. 62 depicts a DART TOF-MS spectrum of a boswellia species extraction process using silica gel 633, stage 3.

FIG. 63 depicts identified compounds in the boswellia species extraction process using silica gel 633, stage 3.

FIG. 64 depicts a DART TOF-MS spectrum of a boswellia species extraction process using silica gel 635, stage 1.

FIG. 65 (A-B) depicts identified compounds in the boswellia species extraction process using silica gel 635, stage 1.

FIG. 66 depicts a DART TOF-MS spectrum of a boswellia species extraction process using silica gel 635, stage 2.

FIG. 67 (A-B) depicts identified compounds in the boswellia species extraction process using silica gel 635, stage 2.

FIG. 68 depicts a DART TOF-MS spectrum of a boswellia species extraction process using silica gel 635, stage 3.

FIG. 69 (A-B) depicts identified compounds in the boswellia species extraction process using silica gel 635, stage 3.

FIG. 70 depicts a DART TOF-MS spectrum of a boswellia species extraction process using silica gel 645, stage 1.

FIG. 71 (A-B) depicts identified compounds in the boswellia species extraction process using silica gel 645, stage 1.

FIG. 72 depicts a DART TOF-MS spectrum of a boswellia species extraction process using silica gel 645, stage 3.

FIG. 73 (A-B) depicts identified compounds in the boswellia species extraction process using silica gel 645, stage 2.

FIG. 74 depicts a DART TOF-MS spectrum of a boswellia species extraction process using silica gel 645, stage 3.

FIG. 75 depicts identified compounds in the boswellia species extraction process using silica gel 645, stage 3.

FIG. 76 depicts a DART TOF-MS spectrum of a boswellia species extraction and fractionation using SCCEP with TKR-methacrylate, stage 1.

FIG. 77 (A-B) depicts identified compounds in the boswellia species extraction process using SCCEP with TKR-methacrylate, stage 1.

FIG. 78 depicts a DART TOF-MS spectrum of a boswellia species extraction and fractionation using SCCEP with TKR-methacrylate, stage 2.

FIG. 79 (A-C) depicts identified compounds in the boswellia species extraction process using SCCEP with TKR-methacrylate, stage 2.

FIG. 80 depicts a DART TOF-MS spectrum of a boswellia species extraction and fractionation using SCCEP with TKR-methacrylate, stage 3.

FIG. 81 (A-B) depicts identified compounds in the boswellia species extraction process using SCCEP with TKR-methacrylate, stage 3.

FIG. 82 depicts a DART TOF-MS spectrum of a boswellia species extraction and fractionation using SCCEP with TKR-STYR-ABS, stage 1.

FIG. 83 (A-B) depicts identified compounds in the boswellia species extraction process using SCCEP with TKR-STYR-ABS, stage 1.

FIG. 84 depicts a DART TOF-MS spectrum of a boswellia species extraction and fractionation using SCCEP with TKR-STYR-ABS, stage 2.

FIG. 85 (A-C) depicts identified compounds in the boswellia species extraction process using SCCEP with TKR-STYR-ABS, stage 2.

FIG. 86 depicts a DART TOF-MS spectrum of a boswellia species extraction and fractionation using SCCEP with TKR-STYR-ABS, stage 3.

FIG. 87 (A-C) depicts identified compounds in the boswellia species extraction process using SCCEP with TKR-STYR-ABS, stage 3.

FIG. 88 depicts a DART TOF-MS spectrum of a boswellia species extraction and fractionation using SCCEP with TKR-C18-15-100A, stage 1.

FIG. 89 (A-C) depicts identified compounds in the boswellia species extraction process using SCCEP with TKR-C18-15-100A, stage 1.

FIG. 90 depicts a DART TOF-MS spectrum of a boswellia species extraction and fractionation using SCCEP with TKR-C18-15-100A, stage 2.

FIG. 91 (A-C) depicts identified compounds in the boswellia species extraction process using SCCEP with TKR-C18-15-100A, stage 2.

FIG. 92 depicts a DART TOF-MS spectrum of a boswellia species extraction and fractionation using SCCEP with TKR-C18-15-100A, stage 3.

FIG. 93(A-B) depicts identified compounds in the boswellia species extraction process using SCCEP with TKR-C18-15-100A, stage 3.

### DETAILED DESCRIPTION OF THE INVENTION

### Definitions

The articles “a” and “an” are used herein to refer to one or to more than one (i.e., to at least one) of the grammatical object of the article. By way of example, “an element” means one element or more than one element.

As used herein, the term “acetylated boswellic acid sub-fraction” comprises the ether, alcohol, or ethyl acetate soluble boswellic acid compounds obtained or derived from Boswellia species that have an enriched concentration of acetylated boswellic acids. Other chemical constituents of Boswellia may also be present in these extraction fractions.

As used herein, “Boswellia” refers to the oleo-resin plant material derived from the Boswellia species botanical. The term Boswellia is also used interchangeably with Boswellia species and means these plants, clones, variants, spores, etc.

As used herein, the term “Boswellia constituent” shall mean a chemical compound found in Boswellia species and shall include all such chemical compounds identified above, including but not limited to the essential oil chemical constituents, boswellic acids, and polysaccharides.

As used herein, the term “boswellic acid fraction” refers to an extraction product that comprises the ether, alcohol, or ethyl acetate soluble boswellic acid compounds obtained or derived from Boswellia and related species, further comprising, but not limited to, compounds such as acetyl-beta-boswellic acid.

The term “compound” refers to multiples or moles of molecules or one or more compounds. In addition, the term “compound” refers to a chemical constituent possessing distinct chemical and physical properties, whereas “compounds” refer to more than one chemical constituent compound.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “essential oil fraction” comprises lipid soluble, water insoluble compounds obtained or derived from Boswellia and related species including, but not limited to, the chemical compound classified as duva-3,9,13-trien-1,5alpha-diol-1-acetate.

As used herein, the term “essential oil sub-fraction” comprises lipid soluble, water insoluble compounds obtained or derived from Boswellia and related species including, but not limited to, the chemical compound classified as duva-3,9,13-trien-1,5alpha-1-acetate having enhanced concentrations of specific compounds found in the essential oil of Boswellia species.

As used herein, “feedstock” generally refers to raw plant material, comprising whole plants alone, or in combination with one or more constituent parts of a plant comprising leaves, roots, including, but not limited to, main roots, tail roots, and fiber roots, stems, bark, leaves, seeds, oleo-resin, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to effect the size and physical integrity of the plant material. Occasionally, the term “feedstock” may be used to characterize an extraction product that is to be used as feed source for additional extraction processes.

As used herein, the term “fraction” means the extraction composition comprising a specific group of chemical compounds characterized by certain physical, chemical properties or physical or chemical properties.

As used herein, the term “one or more compounds” means that at least one compound, such as, but not limited to, duva-3,9,13-trien-1,5alpha-diol-1-acetate (a lipid soluble essential oil chemical constituent of Boswellia species), or acetyl-beta-boswellic acid (an ether or alcohol soluble pentacyclic triterpenic acid of Boswellia species) or a polysaccharide molecule (a water soluble-ethanol insoluble chemical constituent of Boswellia species) is intended, or that more than one compound, for example, duva-3,9,13-trien-1,5alpha-diol-1-acetate and acetyl-beta-boswellic acid is intended. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents.

As used herein, the term “polysaccharide fraction” comprises water soluble-ethanol insoluble polysaccharide compounds obtained or derived from Boswellia and related species.

As used herein, the term “profile” refers to the ratios by percent mass weight of the chemical compounds within an extraction fraction or sub-fraction or to the ratios of the percent mass weight of each of the three Boswellia chemical constituent fractions in a final Boswellia extraction composition.

As used herein, the term “purified” fraction or composition means a fraction or composition comprising a specific group of compounds characterized by certain physical-chemical properties or physical or chemical properties that are concentrated to greater than 50% of the fraction's or composition's chemical constituents. In other words, a purified fraction or composition comprises less than 50% chemical constituent compounds that are not characterized by certain desired physical-chemical properties or physical or chemical properties that define the fraction or composition.

A “subject” refers to a primate (e.g. human), bovine, ovine, porcine, rodent, feline or canine.

The term “synergistic” is art recognized and refers to two or more components working together so that the total effect is greater than the sum of the components.

The term “treating” is art-recognized and refers to curing as well as ameliorating at least one symptom of any condition or disorder.

### Compositions

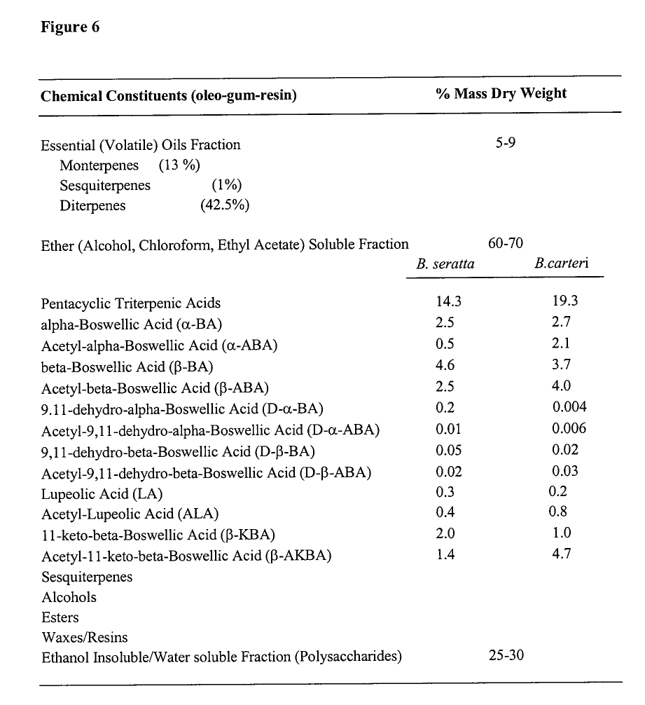

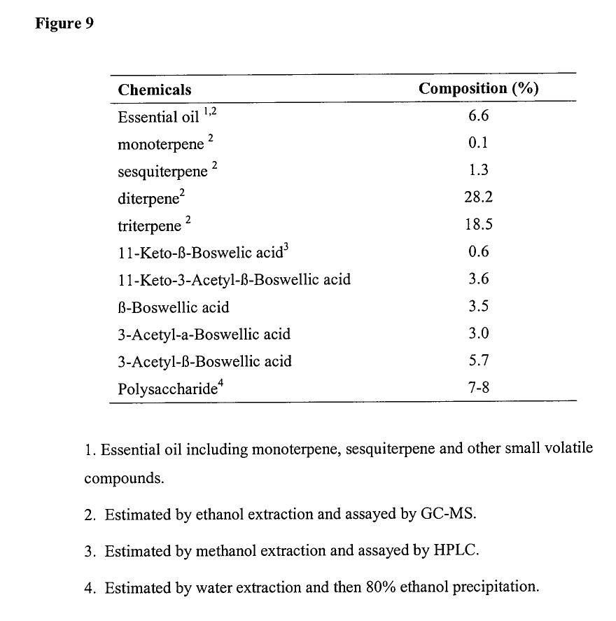

Recent experimental and clinical studies have demonstrated the bioactive properties of the various chemicals and chemical fractions gum resin exudate from the Boswellia species. The principal known bioactive chemicals of the Boswellia species gum resin are listed in FIG. 6 (4-10). In addition, the chemical constituents of B. carteri are listed in FIG. 9 (see Exemplification section).

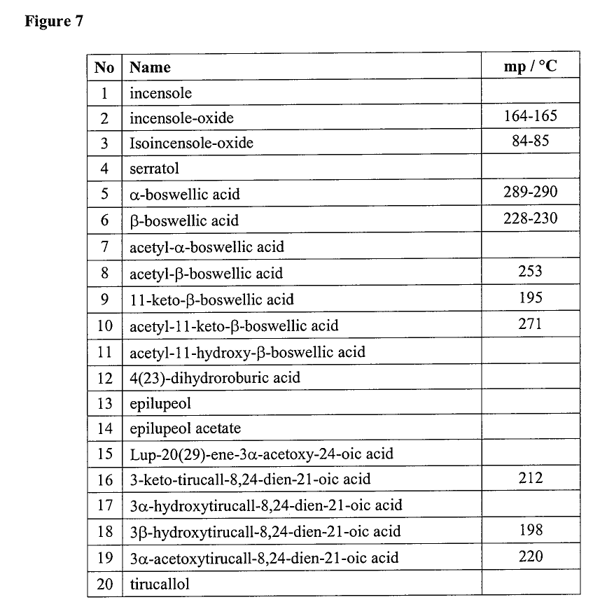

Frankincense is a complex mixture composed of about 5-9% highly aromatic essential oil (monoterpenes and sesquiterpenes), 65-85% alcohol soluble (diterpenes and triterpenes) and water soluble gums that includes polysaccharides. The major monoterpenes are α-pinene, β-myrcene, and limonene. The major sesquiterpenes are β-caryopheyllene, α-capaene, α-humulene, and caryophyllene oxide. The major chemical constituents found in the non-volatile fraction are cambrane and verticillane diterpenes, tetracyclic triterpenes with damarane or titucallane skeletons and pentacyclic triterpenoids belonging to the oleanane, ursane, or lupine groups. α- and β-boswellic acids and their C-acetates are the principal bioactive chemical constituents reported in the majority of scientific papers. The major identified terpenes found in frankincense are shown in FIG. 7.

In brief, the beneficial medicinal activities documented in scientific experimental and clinical studies include the following: anti-inflammatory activities [Boswellic acids fraction-BAF, polysaccharide fraction-PF, essential oil fraction-EOF, extract (1-3, 10-20)]; anti-osteoarthritis, anti-rheumatoid, anti-low back pain, anti-spasmodic, analygesia-effect equal to or greater than non-steroidal anti-inflammatory drugs (NSAIDs) [EOF, BAF, PF [1-3, 10-23)]; anti-bronchial asthma [EOF, BAF, PF, extract (1-3, 10-20, 24,25)]; anti-colitis, anti-ulcerative colitis, anti-Crohn's disease, anti-ileitis [EOF, BAF, PF, extract (1-3, 10-20, 26-29)]; anti-cancer [EOF, BAF, extract (10, 30-34)]; anti-oxidant, anti-atherosclerosis [EOF, BAF, PF, extract (10, 19, 20)]; immunological enhancement [EOF, BAF, extract (10, 35, 36)]; and anti-viral, anti-herpes [EOF, PF, extract (10, 35, 37)]. In addition, Boswellia species extracts are generally consider safe with no known significant toxicities.

Experimental studies of acetyl-boswellic acids, particularly acetyl-11-keto-beta-Boswellic acid (AbBA), indicate potent anti-inflammatory and anti-cancer activities (38-41). In view of this and the relative ease by which Boswellic acids can be purified and isolated, experimental research and commercial extracts have emphasized the acetyl boswellic acids. In fact, currently available commercial extraction products are standardized and labeled as to the content of the boswellic acids ignoring other chemical constituents. Commercially available products may contain 37.5% boswellic acids per dose and some sources claim up to 65% boswellic acids (42).

The present invention comprises compositions of isolated and purified fractions of essential oils (or essential oil sub-fractions), boswellic acids (or boswellic acid sub-fractions), and polysaccharides from one or more Boswellia species. These individual fraction compositions can be combined in specific ratios (profiles) to provide beneficial combination compositions and can provide reliable or reproducible extract products that are not found in currently know extract products. For example, an essential oil fraction or sub-fraction from one species may be combined with an essential oil fraction or sub-fraction from the same or different species or with a boswellic acid fraction or sub-fraction from the same or different species, and that combination may or may not be combined with a polysaccharide fraction from the same or different species of Boswellia.

The present invention comprises compositions that have predetermined amounts of at least one of the essential oil, boswellic acid, or polysaccharide fractions or sub-fractions. Embodiments comprise compositions of Boswellia and related species having at least one of an essential oil, boswellic acid, or polysaccharide fraction concentration that is in an amount greater than that found in the native Boswellia and related species plant material or currently available Boswellia species extract products. Embodiments also comprise compositions wherein one or more of the fractions, including essential oils, boswellic acids, or polysaccharides, are found in a concentration that is less than that found in native Boswellia species.

Compositions of the present invention can comprise a concentration of essential oils in the range of from about 0.001 or 0.01 to 20 times the concentration of native Boswellia species, and/or a concentration of desired boswellic acids in the range of from about 0.001 or 0.01 to 20 times the concentration of native Boswellia species, and/or a concentration of water soluble-ethanol insoluble polysaccharides in the range of from about 0.001 or 0.01 to 20 times the concentration of native Boswellia species. Furthermore, compositions of the present invention comprise sub-fractions of the essential oil chemical constituents having at least one or more of chemical compounds present in the native plant material essential oil that is in amount greater or less than that found in native Boswellia plant material essential oil chemical constituents.

For example, the chemical compound, verticiol, may have its concentration increased in an essential oil sub-fraction to 6.29% by % mass weight of the sub-fraction from its concentration of 3.13% by % mass weight of the total essential oil chemical constituents in the native Boswellia plant material. In contrast, verticiol may have it's concentration reduced in an essential oil sub-fraction to about 0.82% by % mass weight of the sub-fraction from it's concentration of about 3.13% by % mass weight of the total essential oil chemical constituents in the native plant material, a 10-fold decrease in concentration.

Compositions of the present invention may comprise compositions wherein the concentration of specific chemical compounds in such novel essential oil sub-fractions is either increased by about 1.1 to about 5 times or decreased by about 0.1 to about 5 times that concentration found in the native Boswellia essential oil chemical constituents. In addition, compositions of the present invention can comprise fractions of the boswellic acid chemical constituents wherein the concentration of the boswellic acids are increased as a % mass weight of the fraction greater than that found in the native Boswellia species or conventional extraction products.

For example, the concentration of the boswellic acids may be increased in a purified boswellic acid fraction to about 84% by mass weight of the fraction compared to a concentration of about 14-19% by mass weight in the native Boswellia species feedstock, a 4-fold increase in purity of the Boswellic acids. Moreover, the concentration of the acetylated forms of the boswellic acids may be increased in an acetylated and purified acetylated boswellic acid fraction, resulting in a novel boswellic acid profile. For example, the concentration ratio by % mass weight of E-KBA to β-AKBA may be increased from about 1-2:4 in native Boswellia species olibanum resin feedstock or conventional extraction products to about O-trace: 13 in an acetylated purified boswellic acid fraction.

Compositions of the present invention can comprise combinations of one or more extraction compositions taught herein. Such extracted Boswellia species compositions may comprise any one, two, or all three of the concentrated extract fractions depending on the beneficial biological effect(s) desired for the given product.

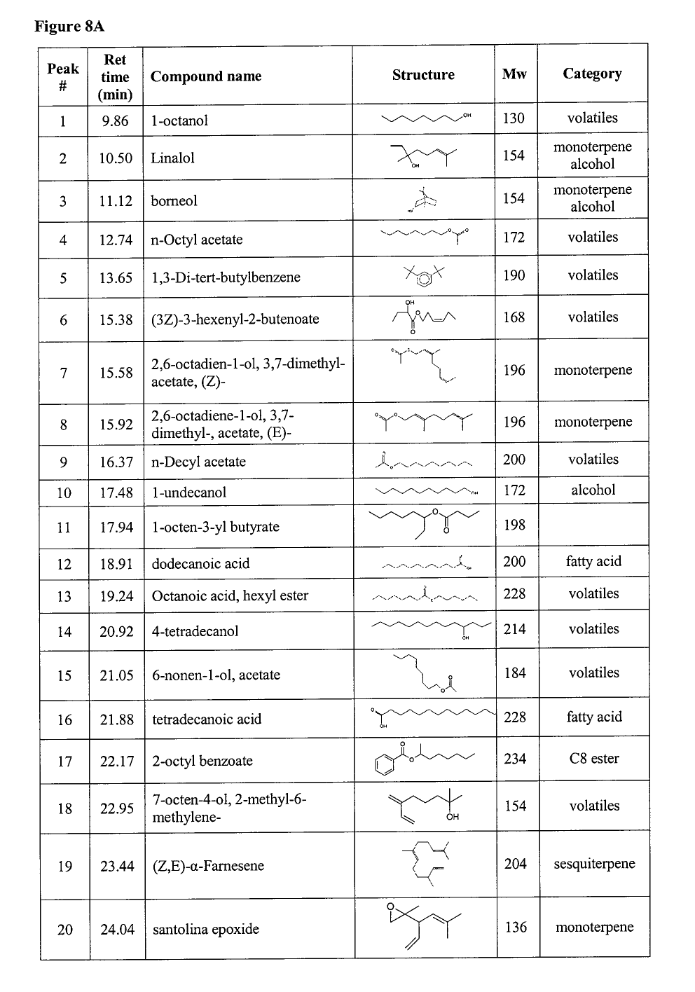

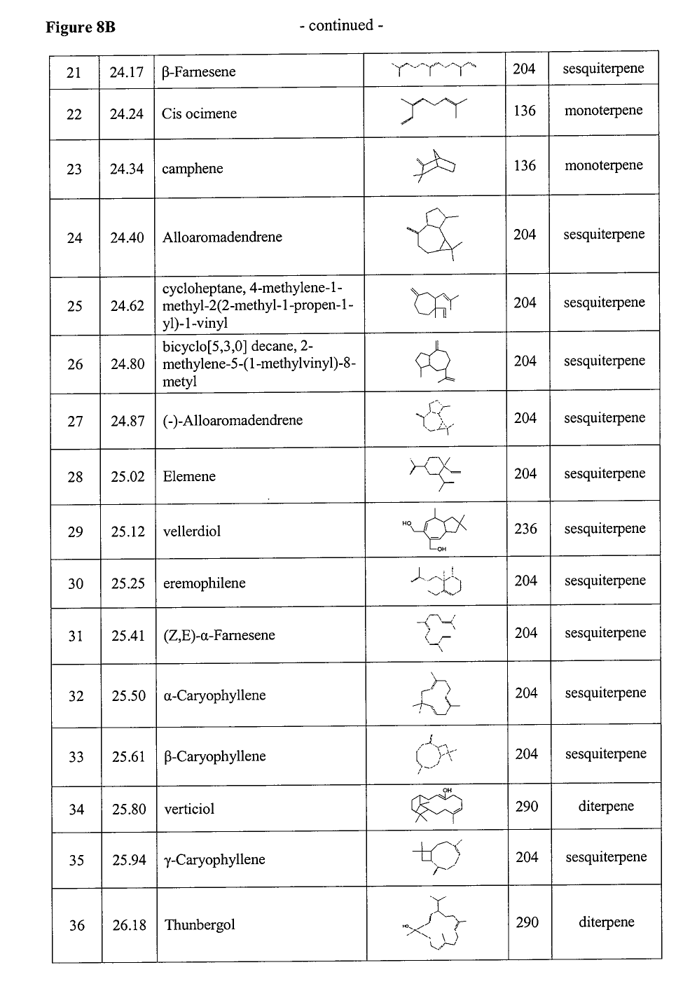

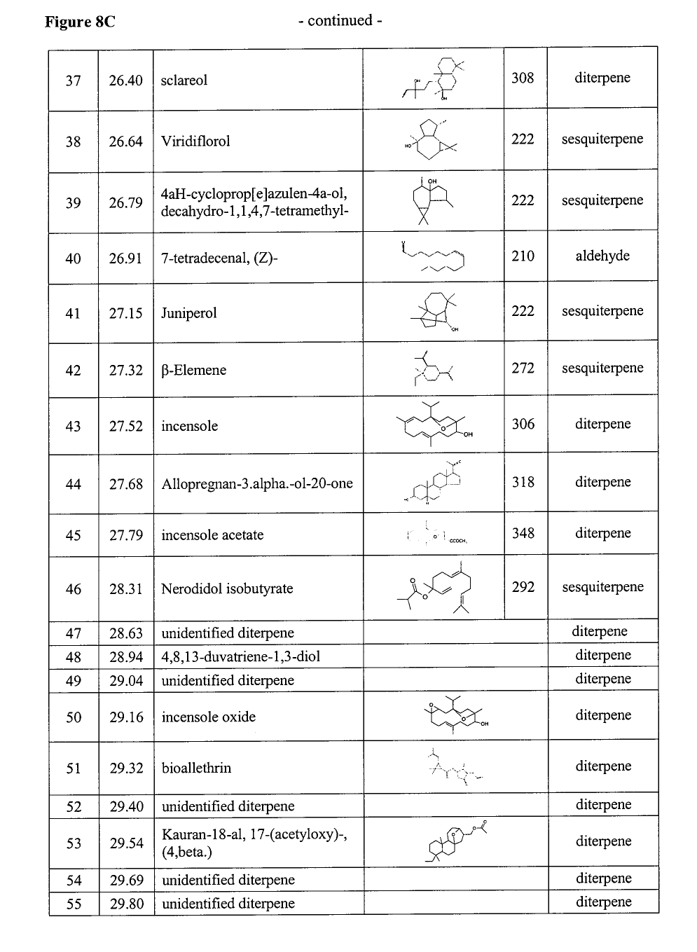

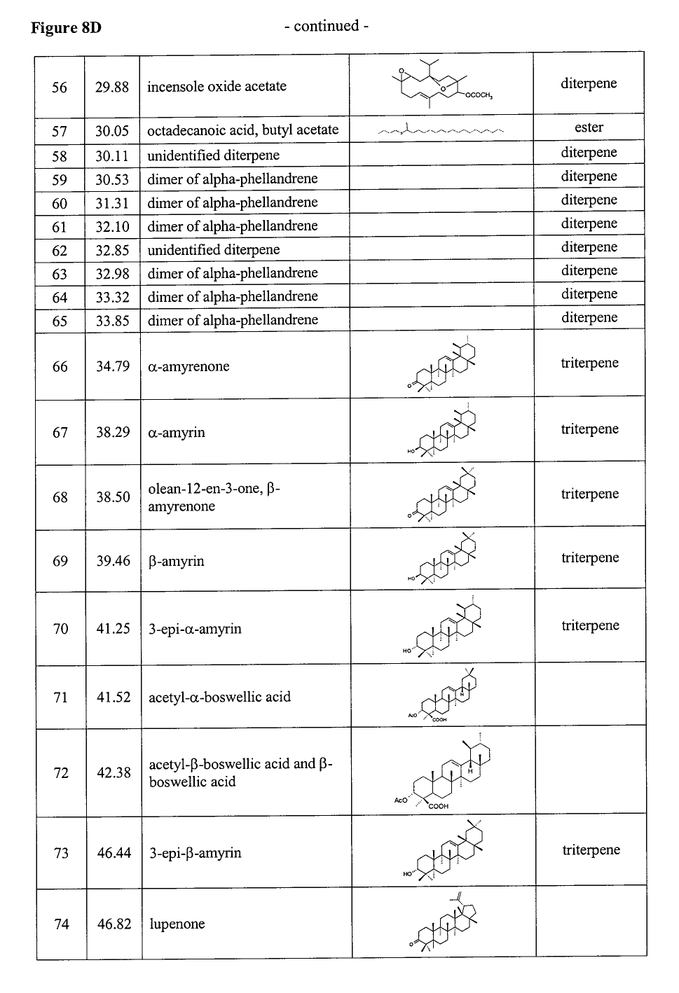

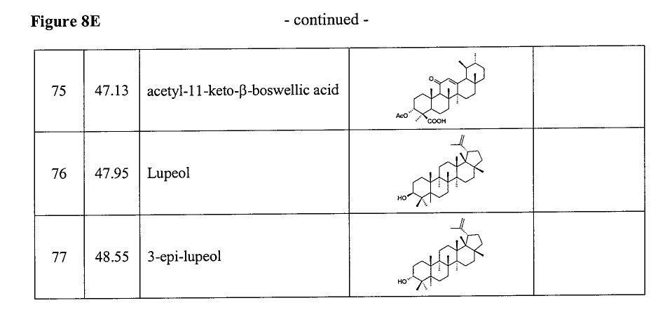

One embodiment comprises a Boswellia essential oil fraction having the components, as shown by GC-MS chromatograms in FIG. 8.

Another embodiment comprises a boswellic acid fraction having the components and GC-MS retention time peaks of about 13.7 (1,3-di-tert-butylbenzene), 17.5 (1-undecanol), 18.9 (dodecanoic acid), 20.9 (4-tetradecanol), 26.6 (viridiflorol), 27.3 (O-elemene), 27.5 (incensole), 28.3 (nerodidol isobutyrate), 29.0 (unknown 2, diterpene), 29.2 (incensole oxide), 30.1 (octadecanoic acid, butyl acetate), 32.1 (dimer of alpha-phellandrene), 34.8 (alpha-amyrenone), 39.5 (beta-amyrin), 41.3 (3-epi-alpha-amyrin), 41.5 (acetyl-alpha-boswellic acid), 42.4 (acetyl-beta-boswellic acid & beta-boswellic acid), 46.4 (3-epi-beta-amyrin), 46.8 (lupeenone), 47.1 (acetyl-11-keto-beta-boswellic acid), 48.0 (lupeol), and 48.6 (3-epi-lupeol) minutes using the GC-MS analytical methods as taught in the present invention and/or the components as shown by HPLC with retention times of about 15.1 (β-KBA), 20.1 (Unknown 1), 26.7 (β-AKBA), 30.5 (Unknown 2), 34.9 (β-BA), 36.5 (Unknown 3), 47.1 (α-ABA), and 49.1 (β-ABA) using the reference standards and the HPLC analytical methods as taught in the present invention.

A third embodiment of a composition comprises an acetylated boswellic acid fraction having the components and GC-MS peak times of about 27.5 (incensole), 27.8 (incensole acetate), 28.3 (nerodidol isobutyrate), 29.2 (incensole oxide), 29.4 (unidentified diterpene), 41.2 (3-epi-α-amyrin), 41.5 (acetyl-α-boswellic acid), 42.4 (acetyl-β-boswellic acid and β-boswellic acid), 46.4 (3-epi-β-amyrin), 46.8 (lupenone), 47.1 (acetyl-11-keto-β-boswellic acid), and 48.6)(3-epi-lupeol) using the GC-MS analytical methods as taught in the present invention and/or the components as shown by HPLC with retention times of about 15.0 (β-KBA), 20.2 (Unknown 1), 26.5 (β-AKBA), 30.3 (Unknown 2), 33.9 (β-BA), 36.1 (Unknown 3), 46.7 (α-ABA), and 4818 (β-ABA) using the reference standards and HPLC analytical methods and analytical methods as taught in the present invention.

A fourth embodiment of a composition comprises a polysaccharide fraction composition, having a purity of greater than 500 mg/g 5K dextran equivalence based on the colormetric analytical methods as taught in the present invention.

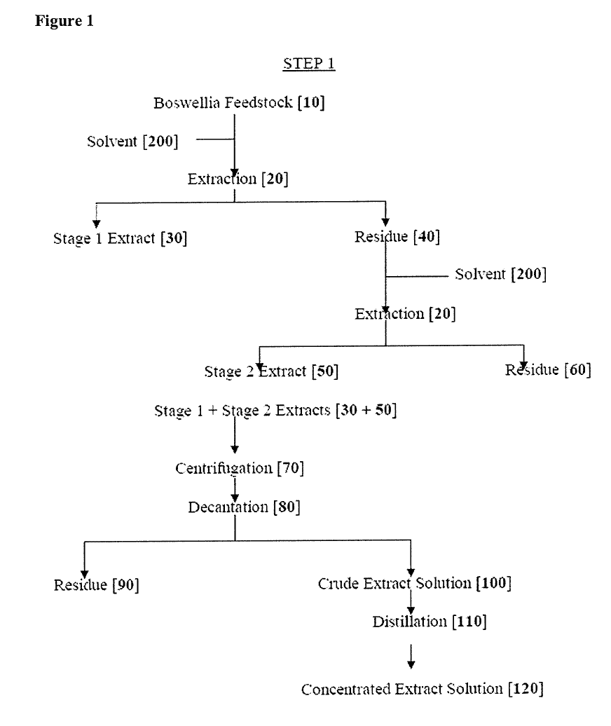

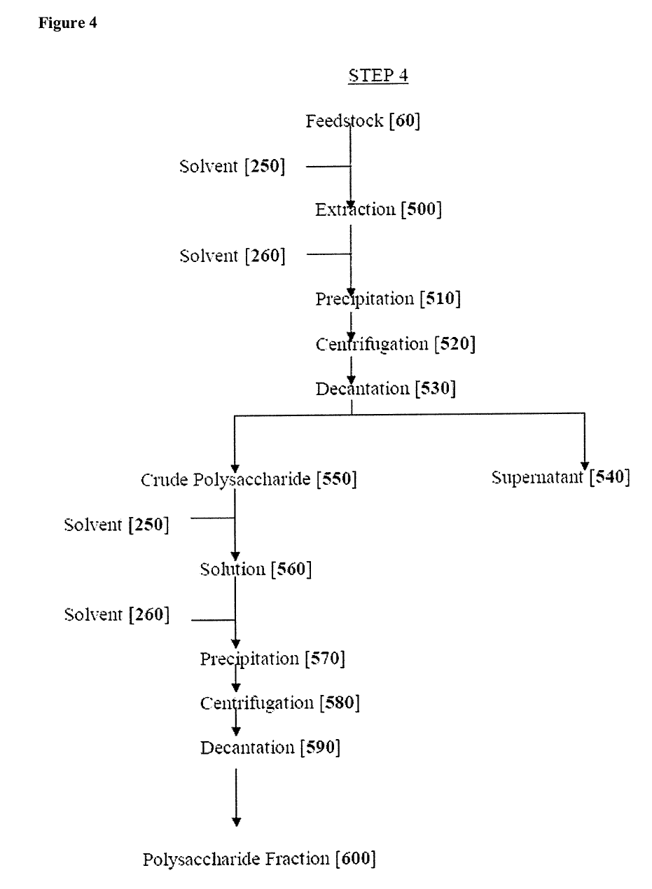

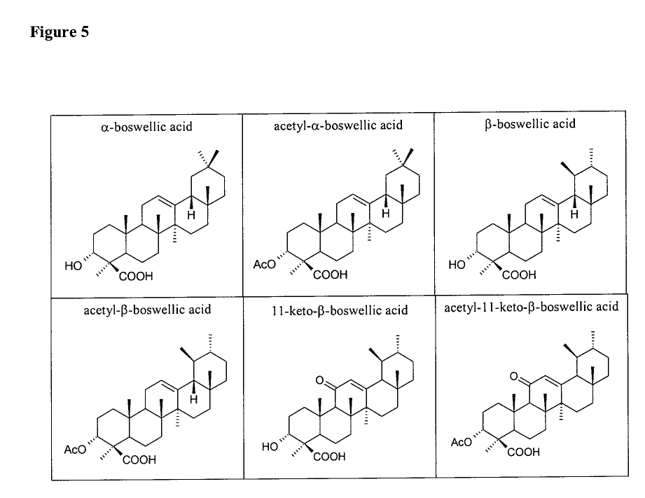

PURITY OF COMPOSITIONS. Using the methods as taught in Step 1 (see FIG. 1) of this invention, an ethanol soluble crude lipophillic fraction is achieved with a 46.9% mass weight yield from the original Boswellia species feedstock having a 98% concentration of total lipid soluble Boswellia species chemical constituents. This yield equates to an about 70% yield by mass weight of the lipid soluble chemical constituents found in the native Boswellia oleo-gum-resin feedstock. Moreover, this absolute alcohol solvent extraction Step 1 preserves the valuable water soluble-ethanol insoluble polysaccharide chemical constituents in the residue for subsequent extract (FIG. 4).

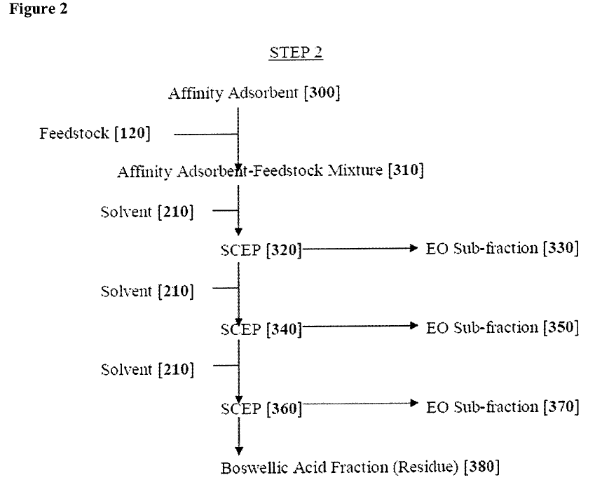

In performing the described extraction methods of Step 2 (see FIG. 2), it was found that greater than 80% yield by mass weight of the essential oil chemical constituents having greater than 95% purity of the essential oil chemical constituents in the original dried Boswellia oleo-gum-resin feedstock of the Boswellia species can be extracted in a single-step or multi-step essential oil SCCEP extract fraction (Step 2). Using the methods as taught in Step 2 (SCCEP Extraction and Fractionation Processes), the essential oil yield was reduced due to the fractionation of the essential oil chemical constituents into highly purified (>90%) essential oil sub-fractions. In addition, the SCCEP extraction and fractionation process as taught in this invention permits the ratios (profiles) of the individual chemical compounds comprising the essential oil chemical constituent fraction to be altered such that unique essential oil sub-fraction profiles can be created for particular medicinal purposes. For example, the concentration of the essential oil chemical constituent element may be increased while simultaneous reducing the concentration of incensole or visa versa. Finally, Step 2 SCCEP processes preserve the triterpenoids, particularly the boswellic acids, in a purified boswellic acid fraction having an about 8% yield by mass weight based on the original Boswellia feedstock with a boswellic acid purity of about 80-86% by mass weight of the extract composition.

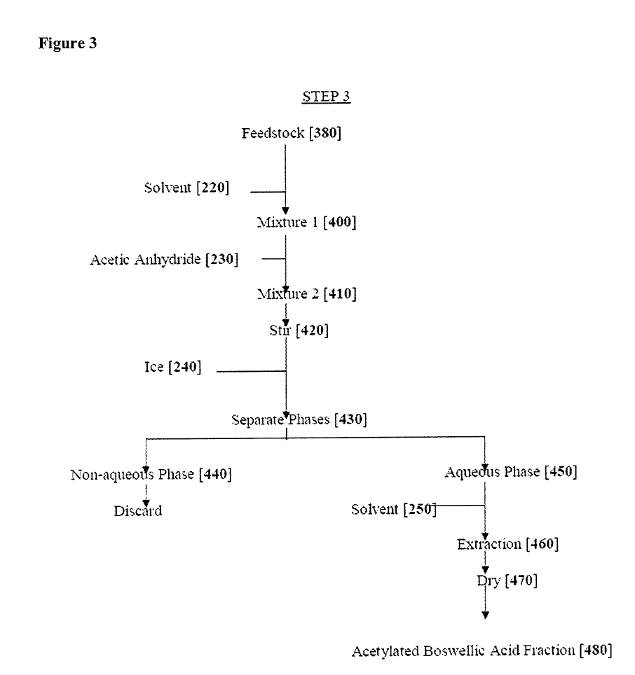

Using the methods as taught in Step 3 (see FIG. 3) of this invention, a purified boswellic acid fraction (boswellic acid concentration of 80-86% by mass weight of the fraction) may be acetylated such that greater than 90% of the non-acetyl boswellic acids may be converted to their acetylated forms with preservation of boswellic acid purity in the acetylated purified boswellic acid fraction at about 80-86% by mass weight of the fraction.

Using the methods as taught in Step 4 (see FIG. 4) of this invention (Polysaccharide fraction extraction and purification), it appears that greater than 95% yield by dry mass weight of the water soluble-ethanol insoluble polysaccharide chemical constituents of the original dried oleo-gum-resin feedstock of the Boswellia species can be extracted in the polysaccharide fraction. Moreover, the purity of the polysaccharide fraction is 500-800 mg/gm dextran standard equivalents using a colormetric analytical method indicating a greater than 95% purity of the purified polysaccharide fraction.

Finally, the methods as taught in the present invention permit the purification (concentration) of the Boswellia species essential oil chemical constituent fractions to be as high as 99% by mass weight of the essential oil chemical constituents, novel essential oil fractions or sub-fractions to be as high as 99% mass weight of the essential oil chemical constituents, a novel boswellic acid fraction to be as high as 87% by mass weight of the triterpenoid chemical constituents, a novel acetylated boswellic acid fraction to be as high as 87% by mass weight of the triterpenoid chemical constituents, and a novel polysaccharide fraction to be as high as 99% by mass weight of the desired chemical constituents in the respective fractions. The specific extraction environments, rates of extraction, solvents, and extraction technology used depend on the starting chemical constituent profile of the source material and the level of purification desired in the final extraction products.

COMPOSITIONS RELATIVE TO NATURAL BOSWELLIA. Embodiments also comprise compositions wherein one or more of the fractions, including the essential oil, the boswellic acids, or polysaccharides, are found in a concentration that is less than that found in native Boswellia plant material. For example, compositions of the present invention comprise compositions where the concentration of the essential oil chemical constituents is from 0.001 to 20 times the concentration of native Boswellia plant material, and/or compositions where the concentration of boswellic acids or acetylated boswellic acids is from 0.0001 to 4 times the concentration of native Boswellia plant material, and/or polysaccharides is from 0.001 to 15 times the concentration of native Boswellia plant material. In making a combined composition, from about 0.001 mg to about 500 mg of a purified essential oil fraction, can be used. Additionally, from about 0.001 mg to about 1000 mg of a purified boswellic acid fraction or purified acetylated boswellic acid fraction can be used. Further, from about 0.001 mg to about 1000 mg of the purified water-soluble ethanol insoluble polysaccharide fraction can be used.

An embodiment of such compositions comprise predetermined concentrations of the extracted and purified chemical constituent fractions wherein the Boswellia species essential oil fraction/boswellic acid or acetylated boswellic fraction, essential oil fraction/polysaccharide fraction, and boswellic acid or acetylated boswellic acid fraction/polysaccharide fraction concentration (% dry weight) profiles (ratios) are greater or less than that found in the natural dried plant material or conventional Boswellia species extraction products.

Alteration of the concentration relationships (chemical profiles) of the beneficial chemical constituents of the individual Boswellia species permits the formulation of unique or novel Boswellia species extract composition products designed for specific human conditions or ailments. For example, a novel and powerful Boswellia composition for anti-inflammatory activity, osteo-arthritis, rheumatoid disease, low back pain disorders, muscular spasm disorders, and analgesia could have a greater purified boswellic acid, particularly acetylated boswellic acids, composition and purified polysaccharide composition and a reduced essential oil composition by % mass weight than that found in the Boswellia native plant material or conventional known extraction products. In contrast, a novel Boswellia composition for immunological enhancement could have a greater purified essential oil composition and purified boswellic acid composition and a reduced polysaccharide composition by % mass weight than that found in the Boswellia native plant material or conventional known extraction products. Another example of a novel Boswellia composition profile for prevention and treatment of colitis, ulcerative colitis, Crohn's disease, and ulcerative ileitis could be a composition profile with greater purified essential oil, boswellic acid composition and purified polysaccharide composition than that found in native Boswellia plant material or known conventional Boswellia extraction products.

SUBFRACTIONS. A further embodiment of the invention is compositions comprising novel sub-fractions of the essential oil chemical constituents wherein the concentration of specific chemical groups such as, but not limited to, volatile oil chemicals and diterpene chemicals have their respective concentrations increased or decreased in novel extraction composition products.

Another embodiment of the invention is compositions comprising novel fractions of the purified boswellic acid chemical constituents wherein the concentration of specific chemical groups such as, but not limited to, acetyl boswellic acids have their respective concentrations increased in novel extraction compositions.

### Methods of Extraction

The following methods as taught may be used individually or in combination with the disclosed method or methods known to those skilled in the art.

The starting material for extraction is plant material from one or more Boswellia species. The plant material may be any portion of the plant, though the oleo-gum-resin is the most preferred starting material.

The Boswellia species plant material may undergo pre-extraction steps to render the material into any particular form, and any form that is useful for extraction is contemplated by the present invention. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the Boswellia species oleo-gum-resin material into a fine powder. The starting material or material after the pre-extraction steps can be dried or have moisture added to it.

SUPERCRITICAL FLUID EXTRACTION OF BOSWELLIA. In general, methods of the present invention comprise, in part, methods wherein Boswellia species is extracted with a solvent extraction step such as, but not limited to, alcohol, ether, acetone, hexane, benezene, ethyl acetate, chloroform, dichloromethane, and the like or their mixtures that is followed by supercritical fluid extraction (SFE) with carbon dioxide as the SFE solvent (SCCO₂) and one or more solvent extraction steps such as, but not limited to, water, hydroalcoholic, and affinity polymer adsorbent extraction processes. Additional other methods contemplated for the present invention comprise extraction of Boswellic species plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, compositions of the present invention may be prepared by a method comprising the steps depicted schematically in FIGS. 1-4.

The invention includes processes for concentrating (purifying) and profiling the essential oil and other lipid soluble compounds from Boswellia plant material using SCCO₂ technology. The invention includes the fractionation of the lipid soluble chemical constituents of Boswellia into, for example, an essential oil fraction of high purity (high essential oil chemical constituent concentration). Moreover, the invention includes a SCCO₂ process wherein the individual chemical constituents within an extraction sub-fraction may have their chemical constituent ratios or profiles altered. For example, SCCO₂ fractional separation of the chemical constituents within an essential oil sub-fraction permits the preferential extraction of certain essential oil compounds relative to the other essential oil compounds such that an essential oil extract sub-fraction can be produced with a concentration of certain compounds greater than the concentration of other compounds.

Extraction of the essential oil chemical constituents of the Boswellia species with SCCO₂ as taught in the present invention eliminates the use of toxic organic solvents and provides simultaneous fractionation of the extracts. Carbon dioxide is a natural and safe biological product and an ingredient in many foods and beverages.

Because Boswellia oleo-gum-resin is a complex mixture of lipophilic substances such as the terpenes, SCCO₂ is the best method for extracting these chemical constituents. It is non-toxic, allows supercritical extractions at relatively low pressures and low temperatures, and solvent removal from the extraction product is not necessary. In general, SCCO₂ behaves like lipophilic solvent but when compared to liquid solvents, it has the advantage that its selectivity or solvent power is adjustable and can be set to conditions that range from gas-like to liquid-like properties. However, the Boswellia oleo-gum resin is highly viscous and sticky, clogging extraction tubing, and thus, making it difficult to extract directly from raw feedstock material using SFE technologies. Therefore, a novel SCCO₂ extraction process entitled “supercritical chromatographic extraction process (SCCEP)” was developed for the present invention. The first step in this process is leaching extraction of Boswellia oleo-gum-resin raw feedstock material using an organic solvent such as, but not limited to, ethanol. The second step is to load the crude solvent leaching extract onto an affinity adsorbent such as, but not limited to, silica gel. The third step is staged de-adsorbtion or elution of the chemical constituents using SCCO₂ at different CO₂ densities. Different densities of CO₂ achieved by either altering the SCCO₂ conditions such as pressure and temperature can be used to elute different fractions of the lipophilic substances characterized by different chemical constituent profiles.

Elution of lipophilic chemical constituents such as, but not limited to, the terpenes from the affinity adsorbent using SCCEP is a highly selective process for fractional separation of these chemical constituents. Several affinity adsorbents could be chosen such as silica gel. Kieselgur, cellulose, bentonite, and magnesium silicate, however, silica gel appears to have the best properties for elution of the Boswellia lipophilic molecules since it yields maximum selectivity in terpene separation. For example, the degree of bonding of different chemical compound groups varies depending on the affinity adsorbent used in the process. Using silica gel as the affinity adsorbent, oxygenated terpenes are adsorbed onto the polar SIOH sites as well as the non-polar SiCH₃ sites due to the non-polar parts of the molecule. In contrast, hydrocarbon terpenes and other organic molecules are adsorbed on the non-polar site only forming relatively weak bonds. In a silica gel column saturated with non-polar mobile phase such as CO₂, moderately polar analytes adsorbed by the polar silica surfaces would elute selectively in order of increasing polarity. For more polar compounds, elution could be accelerated by gradient elution with miscible organic solvents of increasing polarity such as ethyl acetate. Therefore, SCCEP can selectively extract or elute these adsorbed chemical constituents from the affinity adsorbent using different SFE conditions.

A schematic diagram of the methods of extraction of the biologically active chemical constituents of Boswellia is illustrated in FIGS. 1-4. The extraction process is typically, but not limited to, 4 steps. For reference in the text, when the bold number appears in brackets [x], the numbers refers to the numbers in the Figures. The analytical methods used in the extraction process are presented in the Exemplification section.

STEP 1: SOLVENT LEACHING EXTRACTION OF BOSWELLIA ESSENTIAL OIL (LIPOPHILIC) CHEMICAL CONSTITUENTS. A generalized description of the first step in the extraction of the lipophilic (essential oil) chemical constituents from Boswellia species plant material using solvent leaching is diagrammed in FIG. 1—Step 1. The feedstock [10] is the native Boswellia species ground oleo-gum-resin. This feedstock may be leaching extracted in one or more stages. The solvent is an organic solvent [200] such as, but not limited to, ethanol. In this method, the Boswellia species feedstock and the extraction solvent are loaded into an extraction vessel [20], heated, and stirred. It may be heated to about 80° C. or to about 60-70° C. The extraction is carried out for about 1-5 hours, for about 2-4 hours, or for about 2-3 hours. The solid residue [40 & 60] is saved for further processing (Step 4). If multiple stages are used, the extraction solutions are combined [30+50]. The extraction solution slurry is centrifuged [70] and/or filtered and decanted [80]. The supernatant extract solution [60] containing the lipophilic chemicals is collected for mass balance, GC-MS assay, and further processing. The supernatant extract [100] is concentrated using distillation [110]. The concentrated extract solution [120] is used for further processing (Step 2). The methods of the invention are further taught by the experimental in Example 1.

STEP 2. SUPERCRITICAL CHROMATOGRAPHIC EXTRACTION PROCESS (SCCEP). As taught herein, purified lipid soluble sub-fraction extraction products from Boswellia and related species may be obtained by contacting a solvent extract of Boswellia feedstock with a solid affinity adsorbent such as, but not limited to, silica gel so as to preferentially adsorb desired chemical constituents contained in the solvent extract onto the affinity adsorbent. The non-bound as well as the bound chemical constituents are subsequently eluted by the SCCEP methods taught herein. Although various eluants may be employed to recover the desired chemical constituents from the solution and adsorbent, in one aspect of the present invention, the eluant comprises carbon dioxide under supercritical conditions (SCCEP).

Preferably, the Boswellia species feedstock material has undergone a one or more preliminary purification process such as, but not limited to, the processes described in Step 1 prior to contacting the chemical constituent containing concentrated solvent extract with the affinity adsorbent material.

Using the affinity adsorbent SCCEP process as taught in the present invention results in highly purified lipid soluble chemical constituents of the Boswellia species that are remarkably free of other chemical constituents that are normally present in natural plant material or in available extraction products. For example, the processes taught in the present invention can result in purified triterpene sub-fractions that contain total triterpene chemical constituents in excess of 85% by dry mass weight.

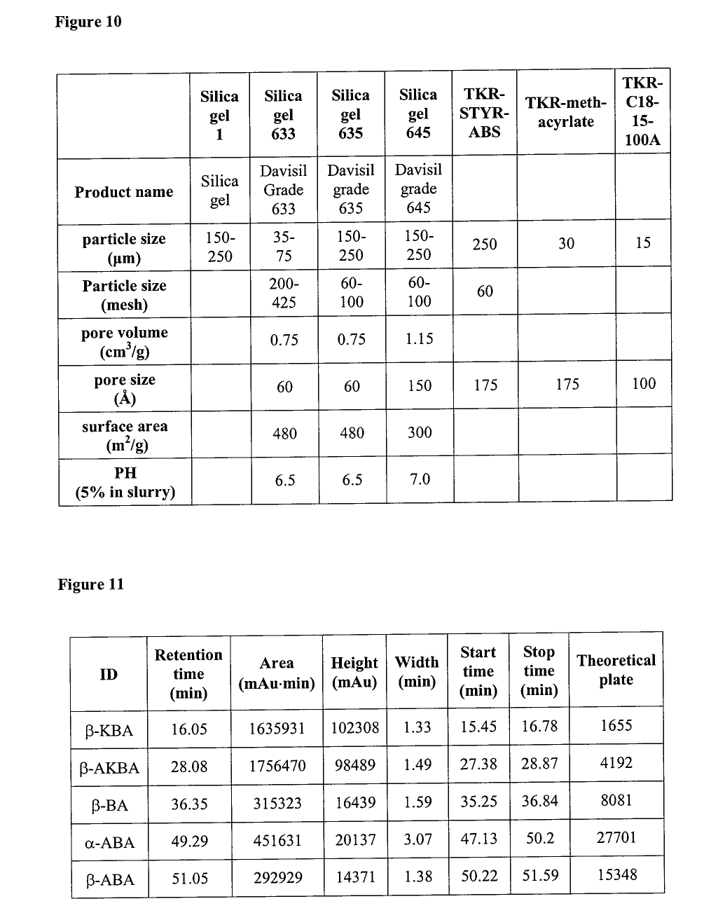

A generalized description of the extraction/fractionation of the essential oil chemical constituents from the concentrated ethanol extract of the Boswellia species using SCCEP is diagrammed in FIG. 2—Step 2. The feedstock [120] is the concentrated crude Boswellia species ethanol extract solution. The appropriate weight of clean affinity adsorbent material (1 gm silica gel/1.33 mL of concentrated 400 mg/mL solvent extract solution) is mixed in a clean vessel. The extraction solvent [210] is pure carbon dioxide. Ethyl acetate or ethanol may be used as a co-solvent. The feedstock mixed with the affinity adsorbent [310] are loaded into a into a SFE extraction vessel [320]. After purge and leak testing, the process comprises liquefied CO₂ flowing from a storage vessel through a cooler to a CO₂ pump. The CO₂ is compressed to the desired pressure and flows trough the feedstock in the extraction vessel where the pressure and temperature are maintained at the desired level. The pressures for extraction range from about 60 bar to 800 bar and the temperature ranges from about 35° C. to about 90° C. The SCCO₂ extractions taught herein are preferably performed at pressures of at least 60 bar and a temperature of at least 35° C., and more preferably at a pressure of about 80 bar to 500 bar and at a temperature of about 40° C. to about 80° C. The time for extraction at each condition (a single stage of extraction) ranges from about 30 minutes to about 2.5 hours, to about 1 hour. The solvent to feed ratio is typically about 50 to 1 for each of the SCCO₂ extractions. The CO₂ is recycled. The extracted, purified, and profiled lipid soluble chemical constituents [330] are then collected in a collector or separator, saved in a light protective glass bottle, and stored in a dark refrigerator at 4° C. The Boswellia feedstock [10] material may be extracted in a one step process (FIG. 2, Step 2 [320]) wherein the resulting extracted and purified Boswellia lipid soluble fraction [330] is collected in a one collector SFE or SCCO₂ system or in multiple fractionation stages (FIG. 2, Step 2 [320→340→360→etc.]) wherein the extracted purified and profiled Boswellia lipid soluble sub-fractions [330, 350, 370, etc.] are separately and sequentially collected in a one collector SFE system. Alternatively, as in a fractional SFE system, the SCCEP extracted Boswellia feedstock material may be segregated into collector vessels (separators) such that within each collector there is a differing relative percentage lipid soluble chemical constituent composition (profile) in each of the purified essential oil sub-fractions collected. The final residue (remainder) [380] is collected, saved and used as a purified boswellic acid (triterpene) fraction composition or for further processing to obtain a purified, highly-acetylated boswellic acid fraction composition (Step 3). An embodiment of the invention comprises extracting the Boswellia species feedstock material using multi-stage SCCO₂ extraction at a pressure of 60 to 700 bar and at a temperature between 35 and 90° C. and collecting the extracted Boswellia material after each stage. A second embodiment of the invention comprises extracting the Boswellia species feedstock material using fractionation SCCO₂ extraction at pressures of 60 to 700 bar and at a temperature between 35 and 90° C. and collecting the extracted Boswellia material in collector vessels at predetermined conditions (pressure, temperature, and density) and predetermined intervals (time). The resulting extracted Boswellia purified lipid soluble sub-fraction compositions from each of the multi-stage extractors or in differing collector vessels (fractional system) as well as the residue containing concentrated boswellic acids can be retrieved and used independently or can be combined to form one or more Boswellia lipid soluble compositions comprising a predetermined lipid soluble chemical constituent concentration that is higher or lower than that found in the native plant material or in conventional Boswellia extraction products. Typically, the total yield of the lipid soluble sub-fractions from Boswellia species using a multi-stage SCCEP extraction in each stage is about 0.5 to about 5.0% by % mass weight based on the original Boswellia species feedstock having a lipid soluble chemical constituent purity of greater than 95% by mass weight of the extract. Moreover, the total yield of the residue boswellic acid fraction following the multi-stage SCEP extraction is about 8-12% by mass weight based on the original Boswellia species feedstock having a triterpene purity of greater than 85% by mass weight of the dry extract. Examples as well as the results of such extraction processes are found below and in FIGS. 9 and 10.

Based on multiple experiments, the extraction yield is highly dependent on CO₂ density and temperature. The highest extraction yields were found at 40° C. and 90 bar, and 80° C. and 160-250 bar. The high extraction yield at 40° C. and 90 bar indicates that these conditions are optimal for maximally extracting the lower molecular weight lipid soluble chemical constituents (the essential oils) and preserving the triterpenes (boswellic acids) in the residue composition.

Based on GC-MS analysis of the lipid soluble extracts and residue, there are four major lipid soluble Boswellic species compound groups found in these extract fractions: 1) Volatile compounds including the small volatile molecules and monoterpenes. Representative compounds of this group include n-octyl acetate and the monterpenes, linalool, borneol, and camphene; 2) Sesquiterpenes, the principal compound are an isomer of elemene and an isomer of caryophyllene; 3) Diterpenes, the major compounds are incensole, incensole acetate, icensole oxide, and incensole oxide acetate; and 4) The triterpene boswellic acids, the major such compounds identified, are the pentacyclic triterpenoids belonging to the oleanane, ursane, and lupine sub-groups.

In the SCCEP Boswellia essential oil fraction or sub-fraction extracts, the major chemical constituents are n-octyl acetate, incensole, incensole acetate, incensole oxide, and incensole oxide acetate. The incensole compounds are macrocylic diterpenoids with a cambrane skeleton are isolated from the neutral fraction of the oleo-resin but were found to be difficult to extract using conventional steam distillation. These findings are consistent with prior report the scientific literature using solvent extraction processes. As taught in the present invention, SCCEP processing can profile the Boswellic species lipid soluble chemical constituents. For example, in a multi-stage SCCEP wherein the temperature is held constant at 40° C., 100 bar may be used to elute the volatile compounds, 150 bar may be used to elute the sesquiterpenes and relatively non-polar diterpenes, and 200 bar used to elute the relatively more polar diterpenes while preserving the triterpenes in the residue, greater than 80% by mass weight of the boswellic acids in the boswellic acid fraction residue. Therefore, SCCEP can profile the Boswellic species lipid soluble chemical constituents in these different extract fractions.

In the SCCEP Boswellia boswellic acid fraction extract (residue), 12 pentacyclic triterpenes were identified by their mass spectrum. SCCEP elution of the triterpenoid compounds was influenced by the number and the type of functional groups. In general, elution decreased with increasing molecular weight of the triterpene. Lupane type triterpenoids were more difficult to elute than the ursane and oleanane isomers. The configuration of C-3 was important for elution with the β-more resistant to elution than the α-configuration. Triterpene compounds of a same sub-group or family eluted according to the following order: 3-α-alcohol, 3-ketone, 3-β-alcohol with carboxylic function at C-24 and last the corresponding O-acetate. Due to the large molecular weight of the triterpenoids, nonpolar CO₂ is not an efficient solvent for extracting the triterpenoids efficiently. Therefore, high concentrations of the desirable triterpenoids are retained in the residue (boswellic acid fraction) with a purity of greater than 83% by mass weight of the extract composition. In order to preserve the triterpenoids in the residue extract composition, mild SFE conditions (temperature of about 40° C. and pressures of less than 200 bar) should be used to elute and fractionate the essential oil chemical constituents.

Similar fractionation results are obtained by using different silica gel affinity adsorbents in the SCCEP processing of the concentrated ethanol leaching extract of Boswellia species. These data confirm the hypothesis that the combination of supercritical CO₂ with silica gel affinity adsorbent chromatography is an excellent technique for extraction and fractionation of the lipid soluble Boswellia species lipid soluble chemical constituents. Of these silica gel adsorbents, silica gel 635, high particle size (150-250 mesh) and surface area (480 m²/gm) is the most optimal for processing the lipid soluble chemical constituents of Boswellia species. Although a higher concentration of Boswellic acids is found in the SCCEP residue of silica gel 635 processing, a significant amount of diterpenes of about 10% by mass weight are also present in the residue. Therefore, consideration of surface modified silica gel affinity adsorbent should be considered for the SCCEP processing of Boswellia species lipid soluble chemical constituents.

In general, a higher purity of the total boswellic acids and triterpenes in the residue (boswellic acid fraction) of SCCEP extraction and fractionation may be achieved by using surface modified silica gel affinity adsorbents. Using surface modified silica gel affinity adsorbents such as TKR-STYR-ABS for SCCEP extraction and fraction of the Boswellia species lipid soluble chemical constituents, it is possible to purify the boswellic acids in the boswellic acid fraction to greater than 80% by mass weight of the fraction. Interestingly, using TKR-styrene-ABS modified silica gel, the major diterpenes, incensole and incensole acetate are processed differently wherein incensole acetate is substantially enriched in the Stage 1 extract fraction and incensole is preferentially enriched in the Stage 2 extract fraction. Although some of the boswellic acids are extracted in the stage 3 fraction by using 5% ethyl acetate as a co-solvent, the use of the co-solvent is important for the removal of polar diterpenes and sesquiterpenes in order to purify the boswellic acids in the boswellic acid fraction (residue).

STEP 3. ACETYLATION OF PURIFIED BOSWELLIC ACID FRACTION. Scientific evidence indicates that the acetylated forms of the boswellic acids exhibit the most potent anti-inflammatory activity and anti-cancer activity. Therefore, the purified boswellic acid fractions obtained from SCCEP processing were acetylated to increase the concentration of boswellic acids having greater beneficial bioactivity.

A generalized description of the acetylation of the purified boswellic acid fraction from obtained from SCCEP of the Boswellica species olibanum is diagrammed in FIG. 3—Step 3. The feedstock [380] for this extraction process may be the purified boswellic acid fraction or residue of SCCEP processing of Step 2 described previously. The feedstock is dissolved in a solvent [220], pyridine, at about 1 gm of feedstock to 10 mL of solvent. To this mixture 1 [400] is added acetic anhydride [230], at about 1 gm of feedstock to about 1.25 gm of acetic anhydride. This mixture 2 [420] is stirred [420] for about 20 hours and then quenched with ice, at about 1 gm of feedstock to about 10 mL of ice [240]. After separation of the phases [240], the aqueous phase [450] was extracted [460] with an organic solvent [250]. The organic solvent may be diethyl either. The organic solvent extraction of the aqueous phase may be performed as many times as deemed necessary to maximize the yield of product. The combined ethyl acetate organic phase is then dried [470] in a vacuum oven. The dried product is the acetylated purified boswellic acid fraction [480].

This process may result in excellent acetylation of the boswellic acids of purified boswellic acid fractions. For example, essentially all of the 11-keto-β-boswellic acid (β-KBA) and β-boswellic acid (β-BA) may be converted to acetyl-11-keto-β-boswellic acid and acetyl-β-boswellic acid, respectively. Although a reference standard could not be obtained for α-boswellic acid HPLC analysis, the high concentration of acetyl-α-boswellic acid in the acetylated purified boswellic acid fractions indicates that essentially all of the α-boswellic acid has been converted to its acetylated form.

STEP 4. POLYSACCHARIDE FRACTION EXTRACTION PROCESSES. The polysaccharide extract fraction of the chemical constituents of Boswellia species has been defined in the scientific literature as the “water soluble, ethanol insoluble extraction fraction”. A generalized description of the extraction of the polysaccharide fraction from extracts of Boswellia species using water solvent leaching and ethanol precipitation processes is diagrammed in FIG. 4—Step 4. The feedstock [60] is the solid residue from the alcoholic leaching extraction process of Step 1. This feedstock is leaching extracted in two stages. The solvent is distilled water [250]. In this method, the Boswellia species residue [50] and the extraction solvent [250] are loaded into an extraction vessel [500] and heated and stirred. It may be heated to 100° C., to about 80° C., or to about 60-80° C. The extraction is carried out for about 1-5 hours, for about 2-4 hours, or for about 2-3 hours. Anhydrous ethanol [260] is then used to make a final solution concentration of 80% ethanol. A precipitation [510] occurs. The solution is centrifuged [520], decanted [530] and the supernatant residue [540] is discarded. The precipitate product [550] is the crude polysaccharide fraction that may be analyzed for polysaccharides using the colormetric method by using Dextran 5,000-410,000 molecular weight as reference standards. The purity of the extracted polysaccharide fraction is about 500 mg/g 5K dextran standard equivalent with a total yield of 25.6% by % mass weight of the original native Boswellia oleo-gum-resin feedstock. For additional purification of the fraction, the precipitate is dissolved in water [250] and then absolute ethanol [260] is added to this solution [560] to make a second 80% ethanol solution to precipitate the purified polysaccharides [570]. This solution is centrifuged [580], decanted [590], and the precipitate saved and dried as the purified polysaccharide fraction [600]. The purify of the final polysaccharide extract fraction is about 800 mg/g 5K dextran standard equivalent with a total yield of about 9.2% by mass dry weight based on the original Boswellia species oleo-gum-resin feedstock. The methods of the present invention are further taught by the experimental example below.

The Boswellia species crude polysaccharide yield was 26.5% by mass weight based on the raw feedstock with a purity of 300-500 mg/g dextran standard equivalents. The purified polysaccharide fraction yield was 9.2% by mass weight based on the raw feedstock with a purity of 500-810 mg/g dextran standard equivalents. These data indicate that the purity of the final polysaccharide fraction composition is greater than 95% with the predominant Boswellia species polysaccharides having molecular weights in the order of 5,000 D. Based on a large number and variety of experimental approaches, it is quite reasonable to conclude that the 9.2% yield is almost 100% of the water soluble-ethanol insoluble polysaccharide present in the natural Boswellia species oleo-gum-resin used in the present invention.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

### Pharmaceutical Formulations

According to a further aspect of the invention, the novel extracted Boswellia species plant material or a novel Boswellia species extract composition can be further processed to dry, flowable powder. The powder can be used as a dietary supplement that can be added to various edible products. The powder or the final predetermined unique extract compositions of the Boswellia species are also suitable for use in a rapid dissolve tablet.

According to a particular aspect of the present invention, the extracted Boswellia species compositions are produced to have a predetermined essential oil, boswellic acids or acetylated boswellic acids, and polysaccharide concentrations that are greater than that found in the natural plant material or conventional Boswellia species extract products and/or predetermined novel profiles of the three major bioactive chemical constituents of the Boswellia species, wherein the ratios (profiles) of the amounts (% dry weight) of essential oil/boswellic acid or acetylated boswellic acid and/or essential oil/polysaccharide and/or boswellic acid or acetylated boswellic acid/polysaccharide are greater or less than the chemical constituent profiles found in the natural Boswellia species plant material or known Boswellia species extraction products. Such novel compositions are particularly well suited for delivery in the oral cavity of human subjects, e.g., via a rapid dissolve tablet.

In one embodiment of a method for producing a Boswellia species extraction powder, a dry extracted Boswellia species composition is mixed with a suitable solvent, such as but not limited to water or ethyl alcohol, along with a suitable food-grade material using a high shear mixer and then spray air-dried using conventional techniques to produce a powder having grains of very small Boswellia species extract particles combined with a food-grade carrier.

In a particular example, an extracted Boswellia species composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spay air-drying. Typical amounts are in the range of between about 5 to about 10 liters per kilogram of extracted Boswellia species material. The solution of extracted Boswellia species composition, maltodextrin and ethyl alcohol is spray air-dried to generate a powder with an average particle size comparable to that of the starting carrier material.

In a second embodiment, an extracted Boswellia species composition and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extracted Boswellia species composition material is combined with food grade material about one and one-half times by weight of the extracted Boswellia species composition, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extracted Boswellia species composition material is then heated until it flows like a heavy oil. Preferably, it is heated to about 50° C. The heated extracted Boswellia species composition is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extracted Boswellia species composition material is introduced into a high shear mixer. The mixture of extracted Boswellia species composition, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extracted Boswellia species composition material, magnesium carbonate, silica and optional emulsifying agent and optional sweetener has an average particle size comparable to that of the starting carrier and a predetermined extraction Boswellia species composition.

According to another embodiment, an extracted Boswellia species composition material is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The Boswellia species extraction composition material is heated until it flows like a heavy oil (preferably heated to 50° C.). The heated Boswellia species extraction composition is then added incrementally to the whey protein and inert carrier that is being mixed in the food processor-type mixer. The mixing of the Boswellia species extraction composition and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of Boswellia species extraction composition, whey protein, and inert carrier is introduced incrementally into the cold water containing high shear mixer while mixing. Sweetening agents or other taste additives of up to 5% by weight can be added at this stage if desired. After mixing is completed, the mixture is dried using freeze drying or refractive window drying. The resulting dry flowable powder of Boswellia species extraction composition, whey protein, inert carrier and optional sweetener has a particle size of about 150 to about 700 micrometers and an unique predetermined Boswellia species extraction composition.

In a further embodiment, a predetermined Boswellia species extraction composition is dissolved in a SFE CO₂ fluid that is then absorbed onto a suitable food-grade carrier such as maltodextrin, dextrose, or starch. Preferably, the SFE CO₂ is used as the solvent. Specific examples include starting with a novel extracted Boswellia species composition and adding from one to one and a half times the extracted Boswellia species material by weight of the food-grade carrier having a particle size of between about 100 to about 150 micrometers. This mixture is placed into a chamber containing mixing paddles and which can be pressurized and heated. The chamber is pressurized with CO₂ to a pressure in the range between 1100 to about 8000 psi and set at a temperature in the range of between about 20 to about 100° C. The exact pressure and temperature are selected to place the CO₂ in a supercritical fluid state. Once the CO₂ in the chamber is in the supercritical state, the Boswellia species extraction composition is dissolved. The mixing paddles agitate the carrier powder so that it has intimate contact with the supercritical CO₂ that contains the dissolved Boswellia species extract material. The mixture of supercritical CO₂, dissolved Boswellia species extraction material, and the carrier powder is then vented through an orifice in the chamber which is at a pressure and temperature that does not support the supercritical state for the CO₂. The CO₂ is thus dissipated as a gas. The resulting powder in the collection vessel is the carrier powder impregnated with the predetermined novel Boswellia species extraction composition. The powder has an average particle size comparable to that of the starting carrier material. The resulting powder is dry and flowable. If needed, the flow characteristics can be improved by adding inert ingredients to the starting carrier powder such as silica up to about 2% by weight as previously discussed.

In the embodiments where the extract composition of the Boswellia species with a predetermined composition or profile is to be included into a oral fast dissolve tablet as described in U.S. Pat. No. 5,298,261 (herein expressly incorporated by reference), the unique extract can be used “neat”, that is, without any additional components which are added later in the tablet forming process as described in the patent cited. This method obviates the necessity to take the unique Boswellia species extract composition to a dry flowable powder that is then used to make the tablet.

Once a dry Boswellia species extraction composition powder is obtained, such as by the methods discussed herein, it can be distributed for use, e.g., as a dietary supplement or for other uses. In a particular embodiment, the novel Boswellia species extraction composition powder is mixed with other ingredients to form a tableting composition of powder which can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339 (herein expressly incorporated by reference), can be used. The tablets can then be removed from the mold and dried, preferably by air-drying for at least several hours at a temperature high enough to drive off the solvent used to wet the tableting powder mixture, typically between about 70° C. to about 85° C. The dried tablet can then be packaged for distribution.

Methods and compositions of the present invention comprise compositions comprising unique Boswellia specie extract compositions in the form of a paste, resin, oil, or powder. An aspect of the present invention comprises compositions of liquid preparations of unique Boswellia species extract compositions. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

An aspect of the present invention comprises compositions of a dry powder Boswellia species extraction composition. Such dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

Although the extraction techniques described herein are discussed in terms of Boswellia species, it should be recognized that compositions of the present invention can also comprise, in the form of a dry flowable powder or other forms, extracts from other plants such as, but not limited to, varieties of gymnemia, turmeric, cinnamon, guarana, cherry, lettuce, Echinacia, piper betel leaf, Areca catechu, muira puama, ginger, willow, suma, kava, horny goat weed, ginko bilboa, mate, garlic, puncture vine, arctic root astragalus, eucommia, gastropodia, and uncaria, or pharmaceutical or nutraceutical agents.

The present invention comprises compositions comprising unique Boswellia species extract compositions in tablet formulations and methods for making such tablets. A tableting powder can be formed by adding about 1 to 40% by weight of the powdered Boswellia species extract composition, with between 30 to about 80% by weight of a dry water-dispersible absorbant such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to 100 mesh.

The present invention comprises compositions comprising tablet formulations and methods for making such tablets. Preferably, the tablet has a formulation that results in a rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed in U.S. Pat. Nos. 5,464,632; 6,106,861; 6,221,392; 5,298,261; 6,221,392; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1 and 1.5%. Additional components include maltodextrin (Maltrin, M-500) at between 1 and 5%. These amounts are solubilized in water and used as a starting mixture to which is added the Boswellia species extraction composition, along with flavors, sweeteners such as Sucralose or Acesulfame K, and emulsifiers such as BeFlora and BeFloraPlus which are extracts of mung bean.

A particularly preferred tableting composition or powder contains about 10 to 60% by of the Boswellia species extract composition powder and about 30 to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used. A sweetener can also be included, preferably in an amount between 3 to about 40% by weight depending on the desired sweetness. Preferred sweetening substances include sugar, saccharin, sodium cyclamate, aspartame, and Stevia extract used singly or in combination, although other sweeteners could alternatively be used. Flavoring such as mint, Boswellia, citrus (e.g., lemon or orange), mocha, and others can be also included, preferably in an amount between about 0.001 to about 1% by weight. If a coloring is desired, natural and/or synthetic colors can be added, preferably in an amount of between about 0.5 to about 2% by weight.

Typically, this tableting composition will maintain its form without the use of a binder. However, if needed, various binders are suitable and can be added in an amount of between about 5 to about 15% or as necessary. Preferred binders are acacia or gum arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM® (available from R. T. Vanderbilt Co., Inc. of Norwalk, Conn.), larch arabogalactan, gelatin, Kappa carrageenan, copolymers of maleic anhydride with ethylene or methyl ether.

A tablet according to this aspect of this invention typically does not require a lubricant to improve the flow of the powder for tablet manufacturing. However, if it is so desired, preferred lubricants include talc, magnesium stearate, calcium stearate, stearic acid, hydrogenated vegetable oils, and carbowax in amount of between about 2% to about homogeneous, the tablet may alternatively be comprised of regions of powdered Boswellia species 100% by weight.

Similarly, a disintegrant does not appear necessary to produce rapid dissolve tablets using the present tablet composition. However, a disintegrant can be included to increase the speed with which a resulting tablet dissolves in the mouth. If desired, between about 0.5% to about 1% by weight of a disintegrant can be added. Preferred disintegrants include starches, clays, cellulose, algins, gums, crosslinked polymers (including croscarmelose, crospovidone, and sodium starch glycolate), VEEGUM® HV, agar, bentonite, natural sponge, cation exchange resins, aliginic acid, guar gum, citrus pulp, sodium lauryl sulphate in an amount of about 0.5% to about 1% of the total mass of the tablet.

It is also generally unnecessary to buffer the tablet composition. However, a buffer may be beneficial in specific formulations. Preferred buffering agents include mono- and disodium phosphates and borates, basic magnesium carbonate and combinations of magnesium and aluminum hydroxide.

In a preferred implementation, the tableting powder is made by mixing in a dry powdered form the various components as described above, e.g., active ingredient (Boswellia species extract composition), diluent, sweetening additive, and flavoring, etc. An overage in the range of about 10 to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the Boswellia species extract composition in the form of a dry flowable powder in a single oral dosage is typically in the range of about 40 to about 1000 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 to about 1,500 mg. In addition to disk, wafer or coin shapes, the tablet can be in the form of a cylinder, sphere, cube, or other shapes. Although the tablet is preferably extract composition separated by non-Boswellia species extract regions in periodic or non-periodic sequences, which can give the tablet a speckled appearance with different colors or shades of colors associated with the Boswellia species extract regions and the non-Boswellia species extract region.

Compositions of unique Boswellia species extract compositions may also comprise Boswellia species compositions in an amount between about 10 mg and about 5000 mg per dose. The essential oil composition of the novel Boswellia species extract composition can vary wherein the essential oil fraction is in an amount between about 0.01 and about 200.0 mg. The total boswellic acid or acetylated boswellic acid fraction composition of the novel Boswellia species extract compositions can vary between about 1 and about 3000 mg per dose wherein the % mass weight of the boswellic acid or acetylated boswellic acid constituents in the unique Boswellia species extraction composition are greater in relation to the % mass weight than that found in the natural Boswellia species plant material or conventional Boswellia species extracts and beverages. The Boswellia species polysaccharide composition of the novel Boswellia species extract composition can vary between about 1.0 and about 2000 mg wherein the % mass weight of the polysaccharide constituents are substantially increased in relation to the % mass weight of polysaccharides found in the natural Boswellia species plant material or conventional Boswellia species extracts or beverages. Furthermore, the % mass weight ratios of the three principal beneficial bioactive chemical constituents (essential oil, boswellic acids or acetylated boswellic acids, and polysaccharides) derived from the Boswellia species may be altered to yield additional novel Boswellia species extract composition profiles for human oral delivery using the doses ranges mentioned previously. Finally, the % mass weight of the individual essential oil chemical constituent compounds may be altered (profiled) to yield novel essential oil fraction composition profiles for human oral delivery using dose ranges as noted. In addition, the purified boswellic acid chemical constituents may be acetylated to yield novel acetylated boswellic acid fraction composition profiles for human oral delivery using dose ranges as noted.

An exemplary 500 mg tablet contains about 300.0 mg powdered predetermine unique Boswellia species extract composition, about 25.0 mg extract of Stevia, about 50.0 mg carboxymethylcellulose, and about 125.0 mg of lactose (see Example 1). An additional exemplary formation for 300 mg Boswellia species extraction composition tablets can be found in Example 2.

The present invention comprises methods of using compositions comprising unique Boswellia species extraction compositions disclosed herein. Methods of providing dietary supplementation are contemplated. Such compositions may further comprise vitamins, minerals and antioxidants. Compositions taught herein can also be used in the methods of treatment of various physiological, psychological, and medical conditions. The standardized, reliable and novel Boswellia species extraction compositions of the present invention are used to prevent and treat type inflammatory disorders, osteo-arthritis, rheumatoid diseases, low back and neck pain, tendonitis, myositis, and muscle spasms. The standardized, reliable, and novel Boswellia species extraction composition can also be used to prevent and treat cardiovascular and cerebrovascular disease. Cardiovascular and cerebrovascular benefits include anti-atherosclerosis and anti-oxidant activity, The novel Boswellia species extraction compositions are used to provide prevention and treatment of colitis, ulcerative colitis, Chrohn's disease, and ileitis. Moreover, the Boswellia species extraction compositions of the present invention are used to provide analgesia. Additional beneficial Boswellia species extract properties include anti-bronchial asthma, immunological enhancement, anti-viral activity including herpes simplex and herpes zoster, and anti-cancer. These and other related pathologies are prevented or treated by administering an effective amount of the novel Boswellia species extraction compositions of the present invention.

The novel Boswellia species extraction compositions may be administered daily, for one or more times, for the effective treatment of acute or chronic conditions. One method of the present invention comprises administering at least one time a day a composition comprising Boswellia species constituent compounds. Methods also comprise administering such compositions more than one time per day, more than two times per day, more than three times per day and in a range from 1 to 15 times per day. Such administration may be continuously, as in every day for a period of days, weeks, months, or years, or may occur at specific times to treat or prevent specific conditions. For example, a person may be administered Boswellia species extract compositions at least once a day for years to enhance mental focus, cognition, and memory, or to prevent and treat type 2 diabetes mellitus, to prevent cardiovascular disease stroke, or to treat gastro-intestinal disorders, or to treat inflammatory disorders and arthritis including gout, or to treat the common cold, bacterial and fungal infections.

### Delivery Systems

Administration modes useful for the delivery of the compositions of the present invention to a subject include administration modes commonly known to one of ordinary skill in the art, such as, for example, powders, sprays, ointments, pastes, creams, lotions, gels, solutions, patches and inhalants.

In one embodiment, the administration mode is an inhalant which may include timed-release or controlled release inhalant forms, such as, for example, liposomal formulations. Such a delivery system would be useful for treating a subject for SARS, bird flu, and the like. In this embodiment, the formulations of the present invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the formulations or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydrofluorocarbons (HFCs) such as 134a and 227. Traditional chlorofluorocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

In another embodiment, the delivery system may be a transdermal delivery system, such as, for example, a hydrogel, cream, lotion, ointment, or patch. A patch in particular may be used when a timed delivery of weeks or even months is desired.

In another embodiment, parenteral routes of administration may be used. Parenteral routes involve injections into various compartments of the body. Parenteral routes include intravenous (iv), i.e. administration directly into the vascular system through a vein; intraarterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is sometimes preferred over oral ones when part of the formulation administered would partially or totally degrade in the gastrointestinal tract. Similarly, where there is need for rapid response in emergency cases, parenteral administration is usually preferred over oral.

### Methods of Treatment

Methods of the present invention comprise providing novel Boswellia compositions for treatment and prevention of human disorders. For example, a novel Boswellia species composition for treatment of arthritis, rheumatic diseases, low back pain, muscle spasm, inflammatory disorders, bronchial asthma, ulcerative colitis and atherosclerosis may have an increased essential oil fraction composition concentration, an increased boswellic acid fraction composition concentration, and an increased polysaccharide fraction composition concentration, by % weight, than that found in the Boswellia species native plant material or conventional known extraction products.

A novel Boswellia species composition for immunological enhancement and for cancer prevention and therapy may have an increased essential oil and boswellic acid fraction composition and a reduced polysaccharide fraction composition, by % weight, than that found in the native Boswellia species plant material or conventional known extraction products. Another example of a novel Boswellia species composition, for treatment of viral disorders such as, but not limited to, herpes simplex and zoster comprises a composition having an increased essential oil fraction composition concentration, an increased polysaccharide composition, and a reduced boswellic acid oil fraction composition than that found in native Boswellia species plant material or known conventional extraction products.

A method of treatment includes methods of treating arthritis comprising administering to a subject in need thereof a therapeutically effective amount of a Boswellia composition of the present invention. In a particularly preferred embodiment, the Boswellia composition further comprises a synergistic amount of similarly obtained extracts of Curcuma species, in particular the Curcuma component curcumin. Methods of extracting Curcuma species are fully described in the provisional patent application filed by the inventors on Sep. 21, 2006, and is hereby incorporated in its entirety. The synergism refers to the increased effect extracts of Boswellia and Curcuma combined have on arthritis compared to the effect each extract has individually.

### EXEMPLIFICATION

The invention now being generally described, it will be more readily understood by reference to the following examples, which are included merely for purposes of illustration of certain aspects and embodiments of the present invention, and are not intended to limit the invention.

### Materials

Botanicals: Boswellia carteri produced in Africa were purchased from Oman, FIG. 9 shows chemical composition measured in house.

Organic Solvents: Ethanol, denatured with 4.8% isopropanol (02853); Ethanol (64-17-5), absolute, (02883); acetonitrile (75-05-8); chromasolv gradient grade for HPLC, ≧99.9% (3451); dichloromethane (75-09-2); pyridine (110-86-1), biotech grade solvent 99% (49445-3); and water (7732-18-5), HPLC grade, (95304). All were purchased from Sigma-Aldrich.

Acids and Bases: Acetic anhydride (108-24-7), min. 98% (A6404); 4-dimethylaminopyridine (DMAP) (1122-58-3); purum, >98% (39450); phenol (108-95-2), purified by redistillation≧99% (328111); phosphoric acid (7664-38-2); ACS reagent, 85 wt % in water (438081); and sulfuric acid (7664-93-9), ACS reagent, 95-98% in water (320501). All were purchased from Sigma-Aldrich.

Chemical Reference Standards: Boswellia standard kit (Kit-00002600-005), including β-boswellic acid (β-BA), acetyl-α-boswellic acid (α-ABA), acetyl-β-boswellic acid (β-ABA), 11-keto-β-boswellic acid (β-KBA) and acetyl-11-keto-β-boswellic acid (β-AKBA), were purchased from Chromadex, Co.

Silica Gel Affinity Adsorbents: Four types of silica gel with different particle size and pore size were used in the present invention. Silica gel 1 (catalog No: 80004) was purchased from Natland International Corporation (www.natland.com). Davisil® Silica gel grade 633 (236772), 635 (236779), and 645 (236837) were purchased from Sigma Aldrich. Surface modified silica gels TKR-STYR-ABS (lot #TKR-06), TKR-C18-15-100A (lot #TKR-03), and TKR-methacrylate (lot #TKR-01) were kindly provided by Technikrom, Inc. The properties are shown in FIG. 10.

### Methods

GAS CHROMATOGRAPHY-MASS SPECTROSCOPY (GC-MS) ANALYSIS. GC-MS analysis was performed at Shimadzu GCMS-QP2010 system. The system includes high-performance gas chromatograph, direct-coupled GC/MS interface, electro impact (EI) ion source with independent temperature control, and quadrupole mass filter. The system is controlled with GC-MS solution Ver. 2 software for data acquisition and post run analysis. Separation was carried out on a Agilent J&W DB-5 fused silica capillary column (30 m×0.25 mm i.d., 0.25 μm film (5% phenyl, 95% dimethylsiloxane) thickness (catalog: 1225032, serial No: U.S. Pat. No. 5,285,774H) using the following temperature program. The initial temperature was 50° C., held for 2 min, then it increased to 250° C. at rate of 8° C./min, then it increased to 320° C. at rate of 3° C./min. No hold time was performed at the upper limit. The total run time was approximately 50 minutes. The sample injection temperature was 250° C. The 1 μl of sample was injected using an auto injector in splitless mode for 1 minute. The carrier gas was helium and the flow rate was controlled by pressure at 55 KPa. Under such pressure, the flow rate was 1.02 mL/min and linear velocity was 36.6 cm/min. MS ion source temperature was 230° C., and GC-MS interface temperature was 300° C. MS detector was scanned between m/z of 40 and 650 at scan speed of 1250 AMU/second with an ionizing voltage at 70 eV. Solvent cutoff temperature was 3.5 min.

HIGH PERFORMANCE LIQUID CHROMATOGRAPHY (HPLC) ANALYSIS. The chromatographic system used was a Shimadzu high Performance Liquid Chromatographic LC-10AVP system equipped with LC10ADVP pump with SPD-M 10AVP photo diode array detector.

The extraction products obtained were measured on a reversed phase Synergi Max-RP column (150×4.6 mm I. D., 5μ, 80 Å) (Phenomenex, Part #: 00G-4053-E0, serial No: 2217520-3, Batch No.: 5243-17). The injection volume was 10 μl and the flow rate of mobile phase was 1 ml/min. The mobile phase consisted of A (0.05% aqueous phosphoric acid, v/v) and B (0.05% phosphoric acid in acetonitrile). The gradient was programmed as follows: with the first 7 minutes, B maintains at 65%, 7-35 min, solvent B increased linearly from 65 to 90%, and 35-43 min, B maintains at 90%, then 43-50 min, B linearly from 90 to 100%, held at 100% for 5 min, and in another 5 min, B linearly from 100 to 65%. β-KBA and β-AKBA were detected at wavelength of 254 nm; β-BA, α-ABA, and β-ABA were detected at wavelength of 210 nm.

Methanol stock solutions of 6 standards were prepared by dissolving weighted quantities of standard compounds into methanol at 5 mg/mL. The mixed reference standard solution was then diluted step by step to a series of solutions at final concentrations of 2, 1, 0.75, 0.5, 0.1 mg/mL, respectively. All the stock solutions and working solution were used within 7 days and stored in +4° C. chiller and brought to room temperature before use. The solutions were used to identify and quantify the compounds in Boswellia. A linear fit ranging from 0.01 to 10 μg was found. The regression equations and correlation coefficients were as follows: β-KBA: peak area/100=116916×C (μg/μl)+1105.1, R²=0.994 (N=6); β-AKBA: peak area/100=126395×C (μg/μl)+1213.1, R²=0.995 (N=6); β-BA: peak area/100=23346×C (μg/μl)+184.8, R²=0.997 (N=6); β-ABA: peak area=area/100=31193×C (μg/μl)+591.3, R²=0.994 (N=6); β-ABA: peak area=area/100=27159×C (μg/μl)−97.2, R²=0.993 (N=6). HPLC analysis results are shown in FIG. 11. The contents of the reference standards in each sample were calculated by interpolation from the corresponding calibration curves based on the peak area.

POLYSACCHARIDE ANALYSIS. The spectrophotometer system used was a Shimadzu UV-1700 ultraviolet visible spectrophotometer (190-1100 nm, 1 mm resolution) has been used in the present invention.

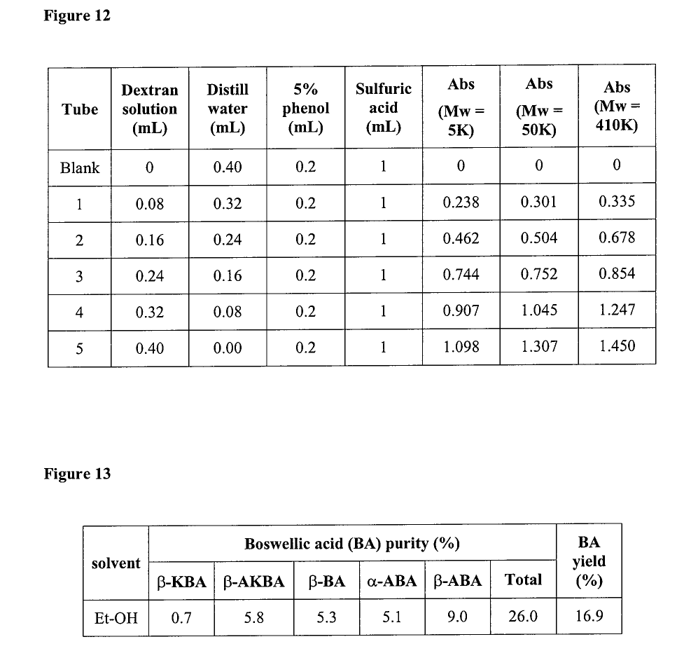

A calorimetric method was used for polysaccharide analysis. Make 0.1 mg/mL stock dextran (Mw=5000, 50,000 and 410,000) solutions. Take 0.08, 0.16, 0.24, 0.32, 0.40 mL of stock solution and make up volume to 0.4 mL with distilled water. Then add in 0.2 mL 5% phenol solution and 1 ml concentrated sulfuric acid. The mixtures were allowed to stand for 10 minutes prior to performing UV scanning. The maximum absorbance was found at 488 nm. Then set the wavelength at 488 nm and measure absorbance for each sample. The results are shown in FIG. 12. The standard calibration curves were obtained for each of the dextran solutions as follows: Dextan 5000, Absorbance=0.01919+0.027782 C (μg), R²=0.97 (N=5); Dextan 50,000, Absorbance=0.0075714+0.032196 C (μg), R²=0.96 (N=5); and Dextan 410,000, Absorbance=0.03481+0.036293 C (μg), R²=0.98 (N=5).

DIRECT ANALYSIS IN REAL TIME (DART) MASS SPECTROMETRY FOR POLYSACCHARIDE ANALYSIS. The Jeol AccuTOF-MS DART settings were loaded as follows: DART Needle voltage=300 V; Electrode 1 voltage=150 V; Electrode 2 voltage=250 V; Temperature=250° C.; He Flow Rate=1.20 LPM.

The following Jeol AccuTOF mass spectrometer settings were loaded: Ring Lens voltage=5 V; Orifice 1 voltage=10 V; Orifice 2 voltage=5 V; Peaks voltage=1000 V (for resolution between 100-1000 amu); Orifice 1 temperature was turned off.

The samples were introduced by placing the closed end of a borosilicate glass capillary tube into the Bosswellia serrata or B. carteri extracts, and the coated capillary tube was passed through the He plasma until signal was observed in the total-ion-chromatogram (TIC). The sample was removed and the TIC was brought down to baseline levels before the next sample was introduced. A polyethylene glycol 600 (Ultra Chemicals, Kingston R.I.) was used as an internal calibration standard giving mass peaks throughout the desired range of 100 to 1000 amu.

The DART mass spectra of each extract was searched against a proprietary chemical database and used to identify many chemicals present in the Bosswellia serrata extracts. Search criteria were held to the [M+H]⁺ ions to within 10 amu of the calculated masses. The identified compounds are reported with greater than 90% confidence.

### Example 1

ETHANOL LEACHING EXTRACTION OF BOSWELLIA LIPOPHILIC CHEMICAL CONSTITUENTS. A typical experimental example of solvent leaching extraction of the lipophilic chemical constituents of Boswellia species is as follows: 100 gm of ground (140 mesh) Boswellia species oleo-gum-resin was extracted in two stages using 1000 mL of absolute ethanol for two hours at 65° C. with stirring. The solvent/feed ratio of each stage was 5:1. After extraction, the two extract solutions were combined and the ethanol slurry was centrifuged at 2000 rpm for 10 minutes. The supernatant was collected as “crude extract” and measured using mass balance, HPLC, and GC-MS. The crude extract was then concentrated using rotary evaporation-distillation and recycling of the ethanol solvent to produce an ethanol solution of 200 mL, a 5-fold increase in concentration of the extract. The concentrated ethanol solution yields a high viscosity optimizing the loading of the solution onto the affinity adsorbent of Step 2 processing. The major identified chemical compounds and their GC-MS peak area results are found in FIG. 8. The results of the HPLC analysis of the boswellic acids in the crude ethanol leaching fraction is shown in FIG. 13.

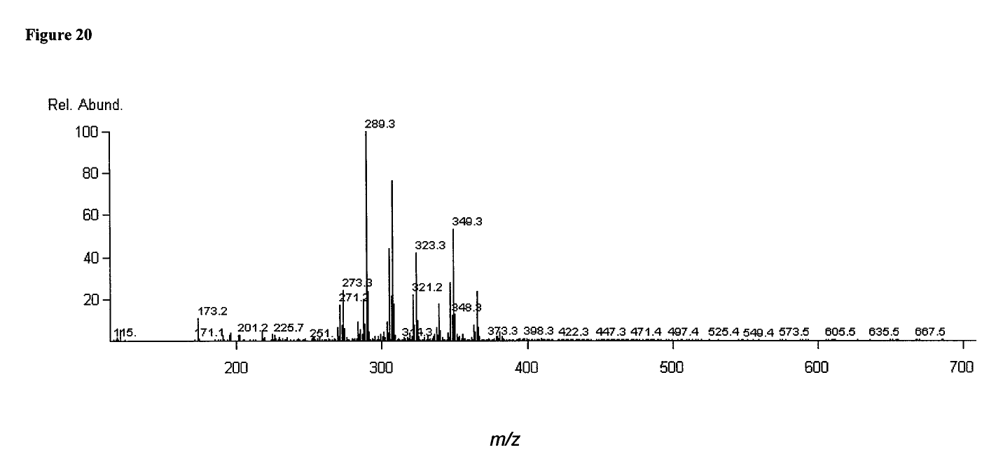

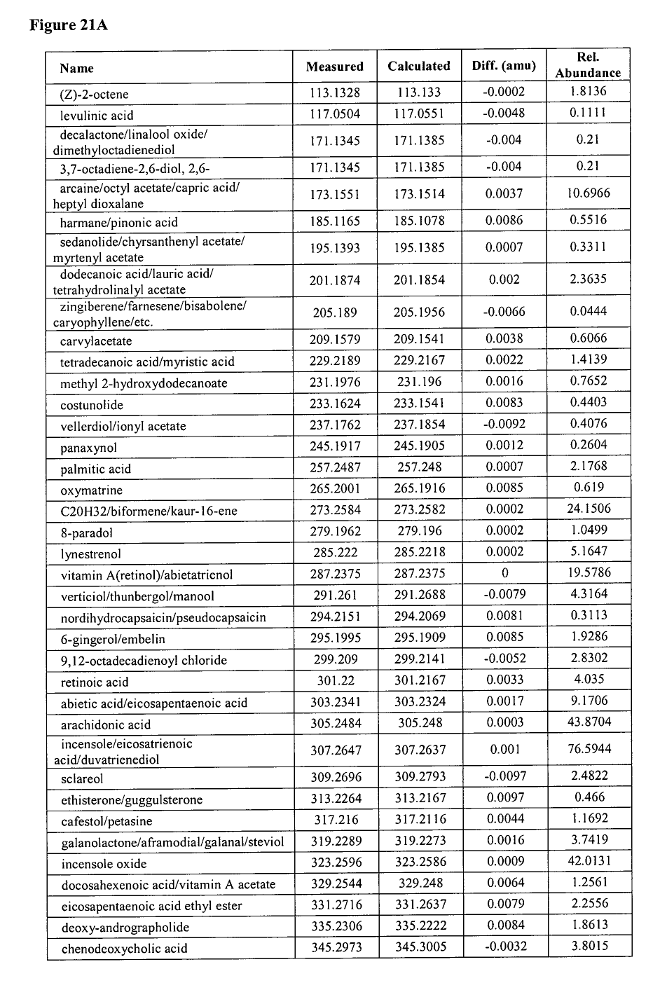

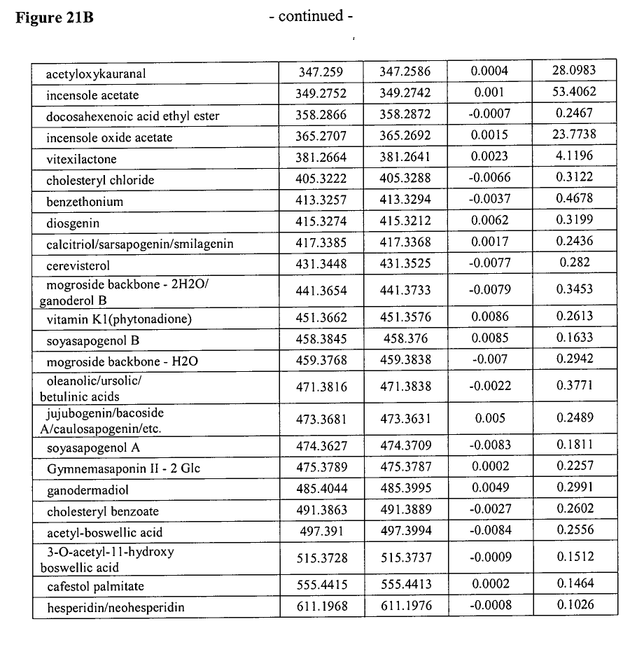

A. 80% ETHANOL EXTRACT OF BOSWELLIA CARTERI AT ROOM TEMPERATURE. Acetyl boswellic acid and 3-O-acetyl-11-hydroxy boswellic acid are present in this extract, but at relatively low abundance. A few of the major compounds (by relative abundance) in HS700 are capric acid, biformene, vitamin A, arachidonic acid, incensole (and incensole derivatives), and acetyloxykauranal. Hesperidin, a higher molecular weight flavonoid (m/z [M+H]+=611.1976), was the only flavonoid identified in HS700. Betulinic acid, 6-gingerol, capsaicin, and ganoderols were also identified in HS700. See FIGS. 20 and 21.

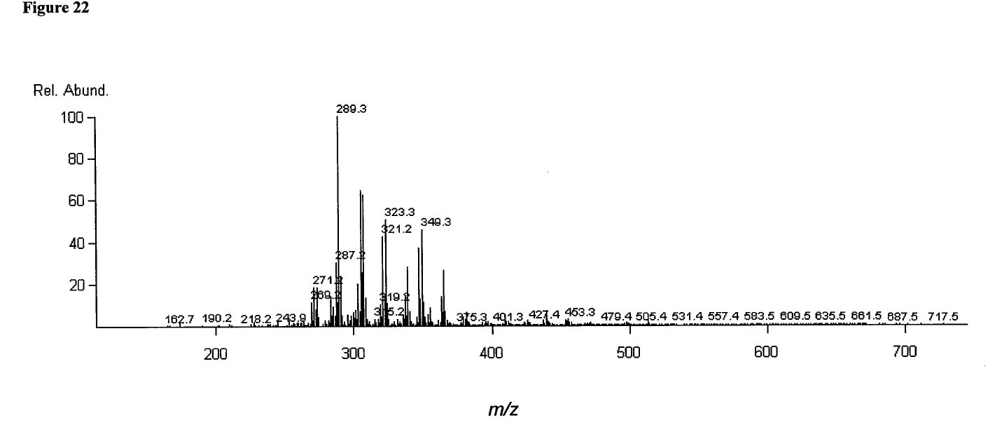

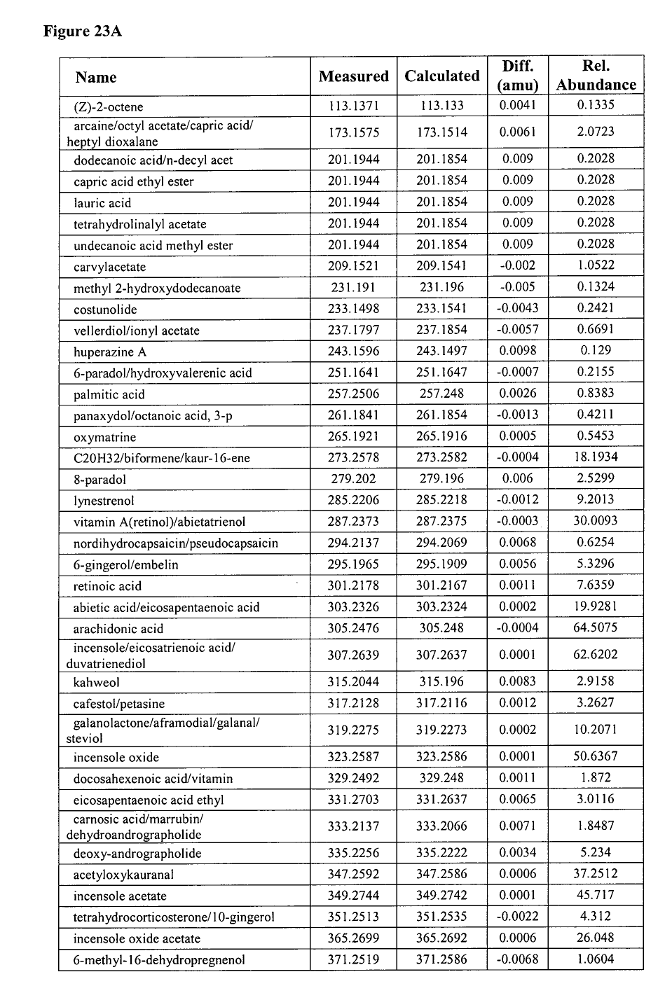

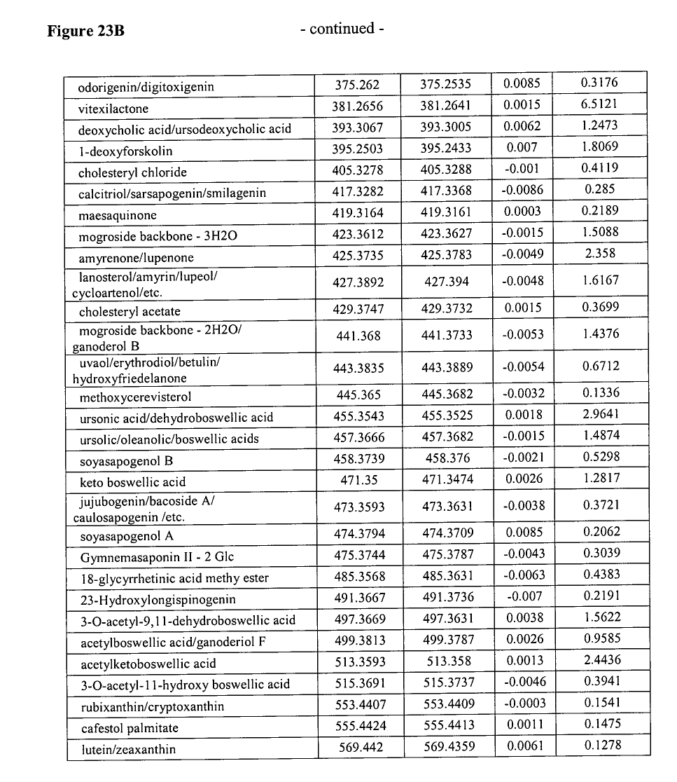

B. 100% ETHANOL EXTRACT OF BOSWELLIA CARTERI AT ROOM TEMPERATURE. The extract had a similar DART TOF-MS profile as the 80% ethanol extract (see A above) in terms of the most abundant compounds identified. The boswellic acids identified in the extract include dehydroboswellic acid (DHBA), boswellic acid (BA), ketoboswellic acid (KBA), 3-O-acetyl-9,11-dehydroboswellic acid (ADHBA), acetylboswellic acid (ABA), acetylketoboswellic acid (AKBA), and 3-O-acetyl-11-hydroxyboswellic acid (AHBA). Lutein was identified at m/z [M+H]+=569.442. See FIGS. 22 and 23.

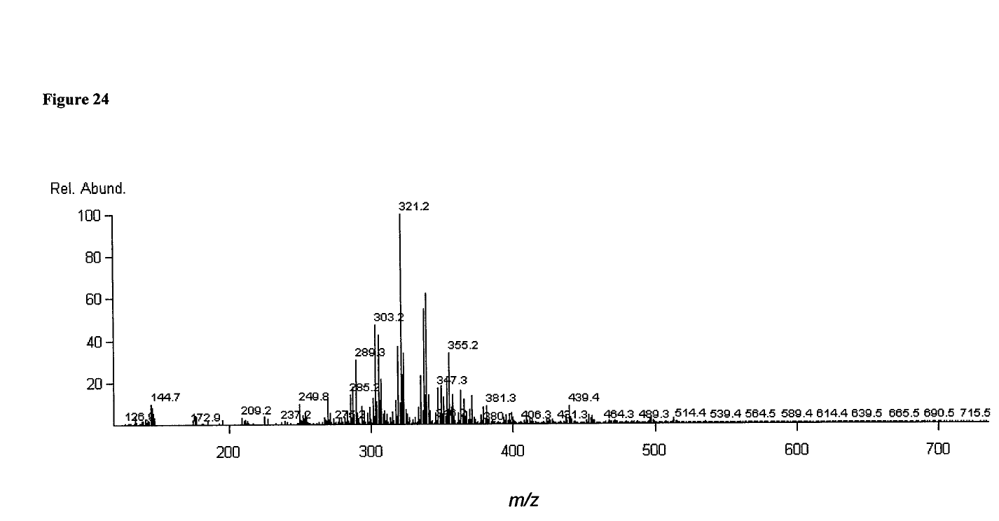

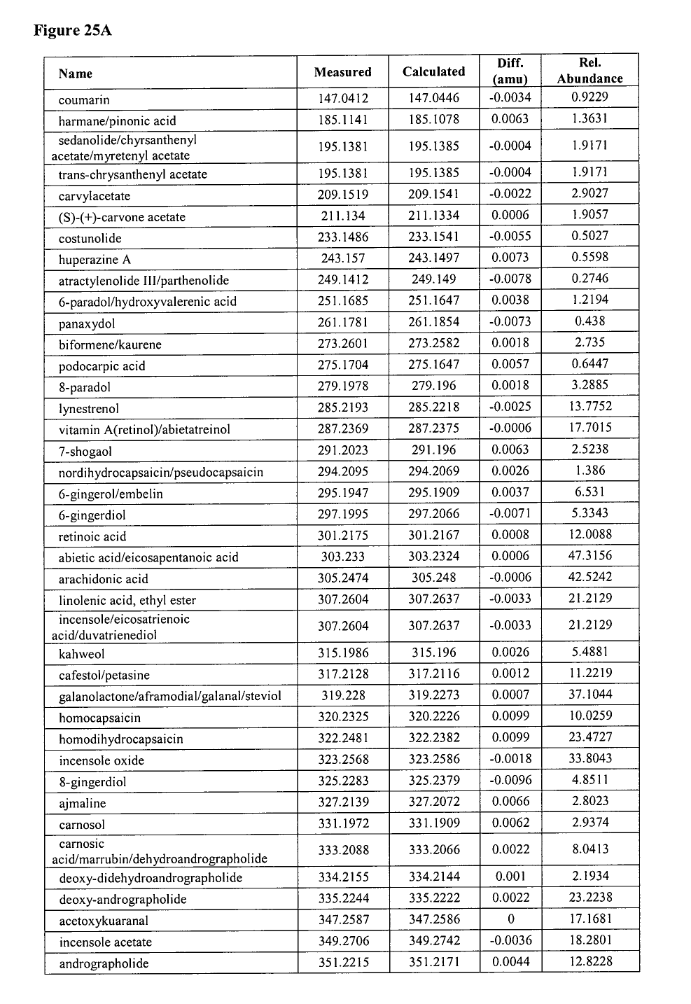

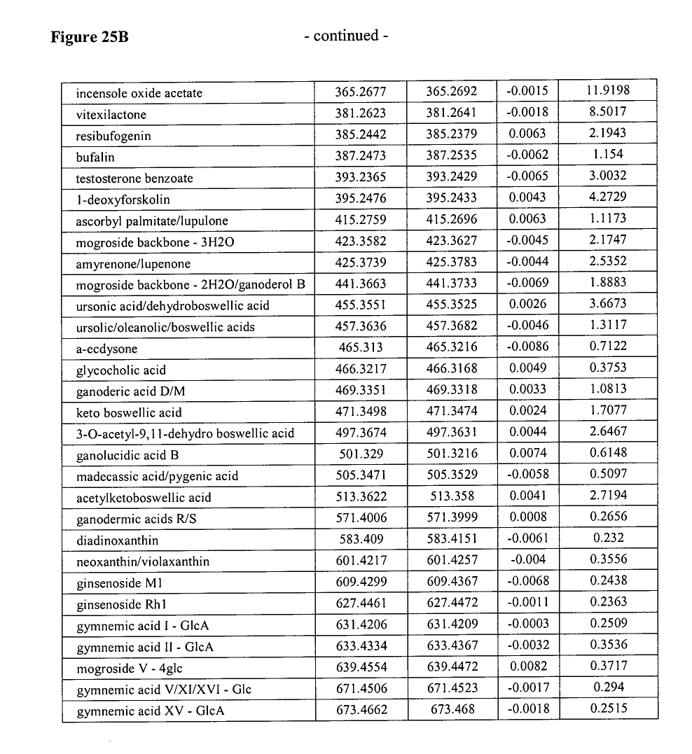

C. DISTILLED WATER EXTRACT OF BOSWELLIA CARTERI AT 40° C. Many gymnemic acids, ginsenosides, gonderderic acids and gingerols were identified in the extract (HS702). The boswellic acids identified in the extract include DHBA, BA, KBA, and AKBA. Coumarin is present in the extract at less than 1% relative abundance. See FIGS. 24 and 25.

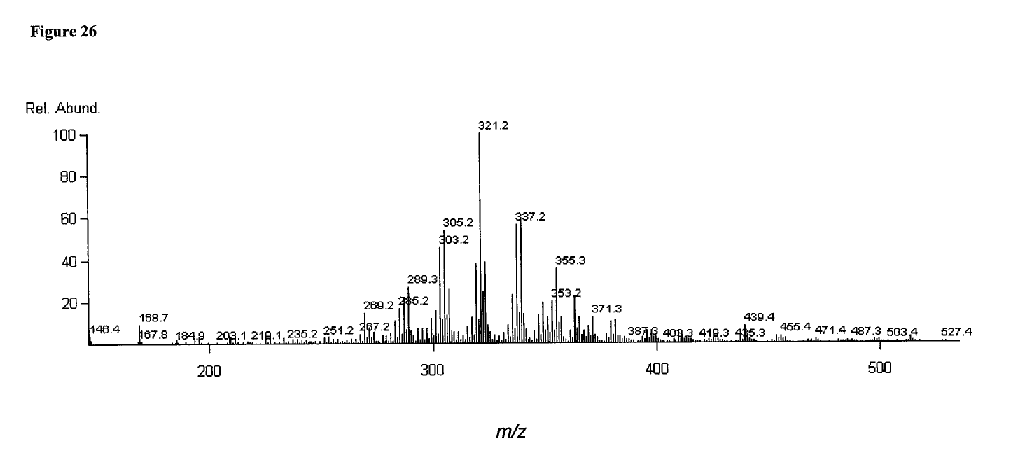

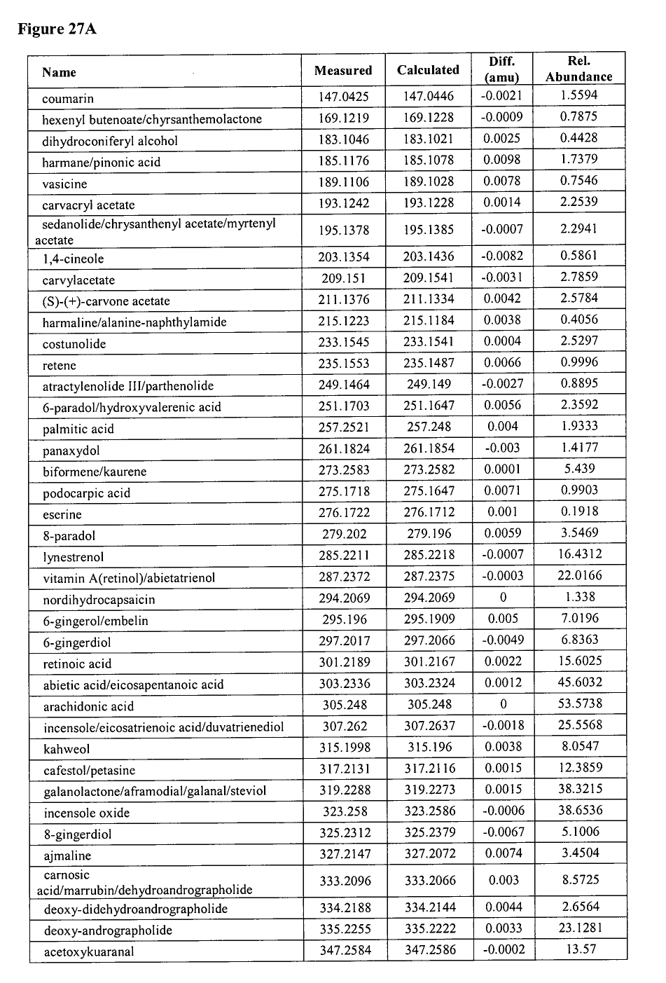

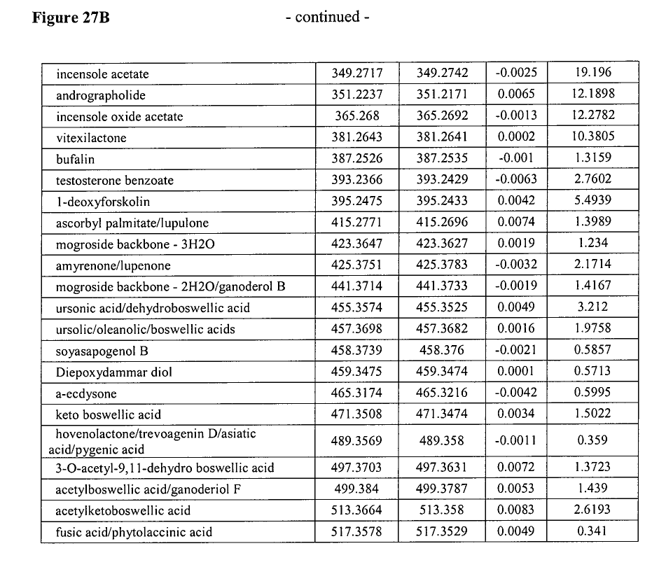

D. 20% ETHANOL EXTRACT OF BOSWELLIA CARTERI AT 40° C. Coumarin is present in the extract in less than 2% relative abundance. Many of the previously discussed compounds are present in the extract including biformene, vitamin A, arachidonic acid, incensole (and incensole derivatives), acetyloxykauranal, norhydrocapsaicin, gingerols, and ganoderols. The boswellic acids identified in the extract include DHBA, BA, KBA, ADHBA, ABA, and AKBA. See FIGS. 26 and 27.

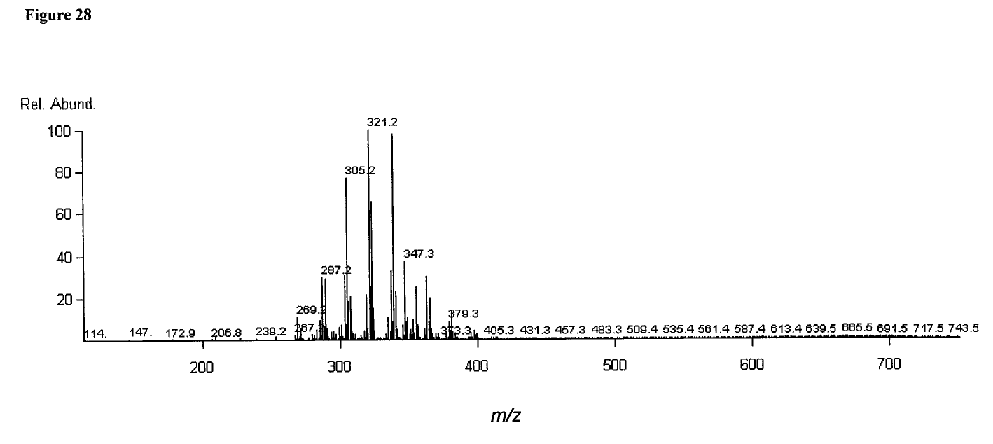

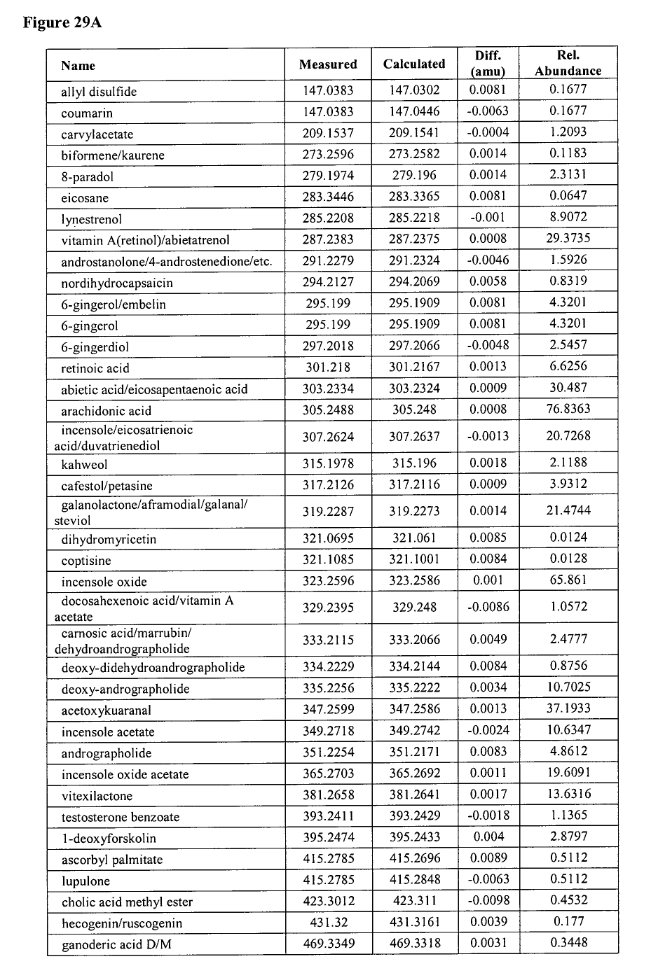

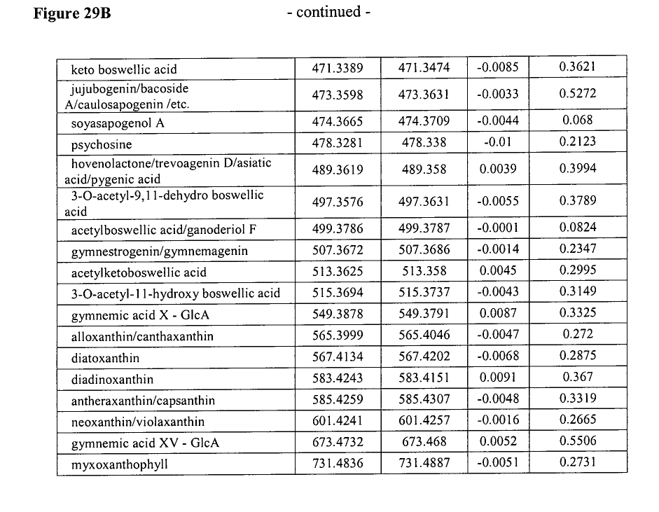

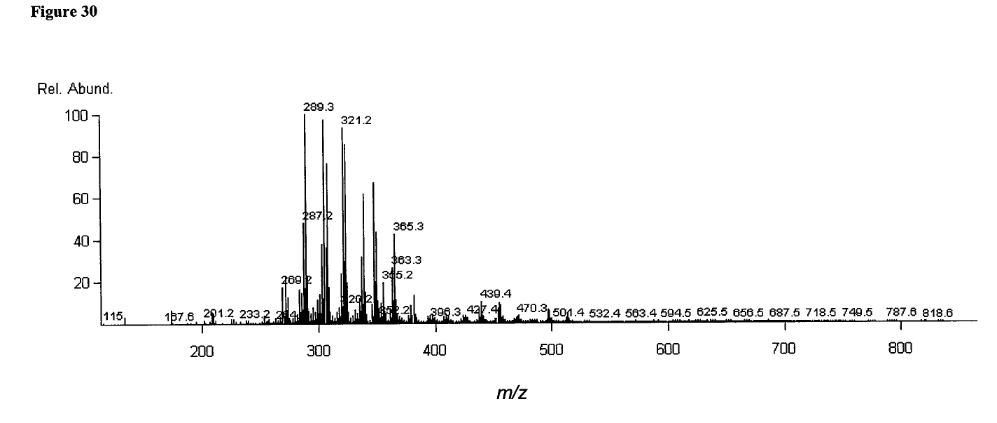

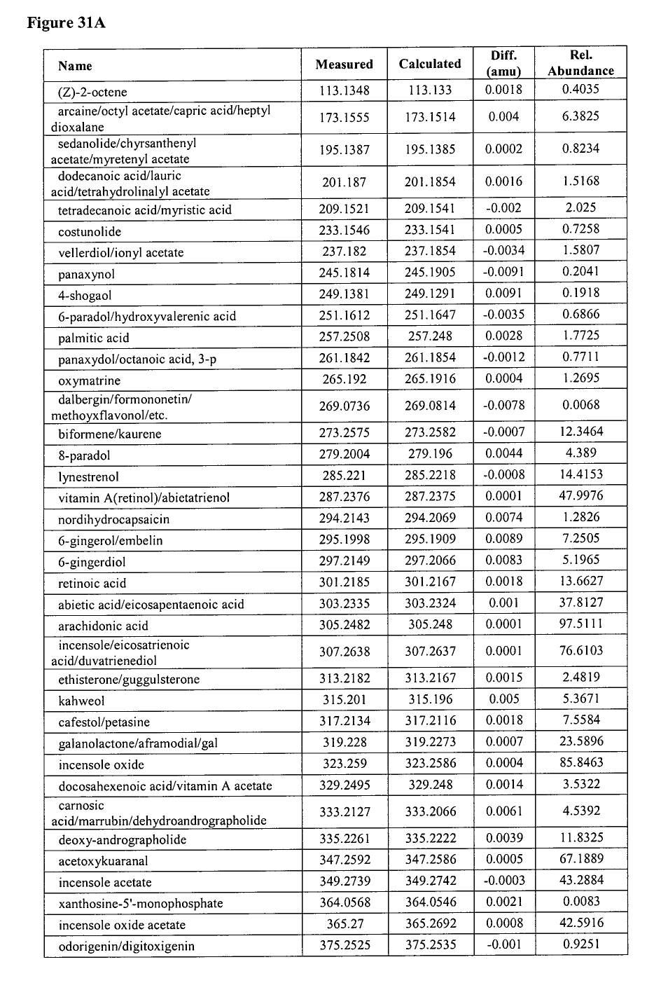

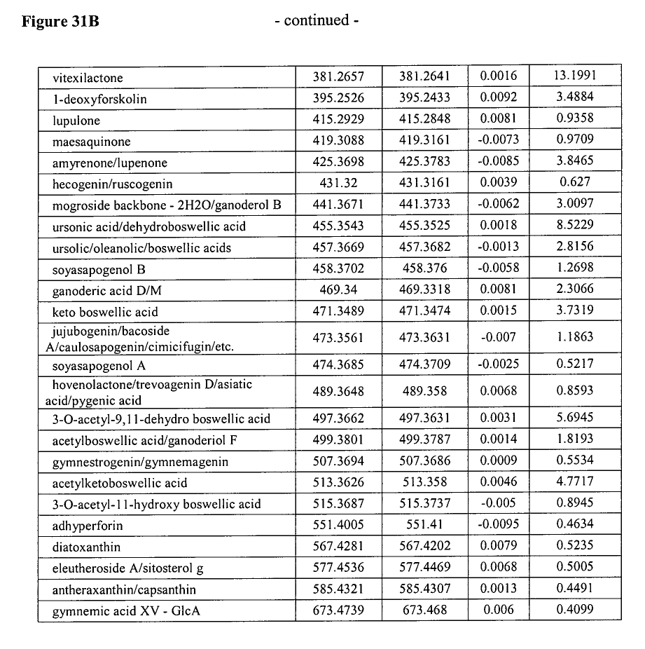

E. 40% ETHANOL EXTRACT (400 C) OF BOSWELLIA CARTERI. Coumarin is present in the extract in less than 1% relative abundance. Many of the previously discussed compounds are present in the extract including biformene, vitamin A, arachidonic acid, incensole (and incensole derivatives), acetyloxykauranal, norhydrocapsaicin, gingerols, gymnemic acids, and ganoderols. The boswellic acids identified in HS704 include KBA, ADHBA, ABA, AKBA, and AHBA. Many xanthin derived compounds have been identified between m/z [M+H]+=565.4 and 731.5. See FIGS. 28 and 29.

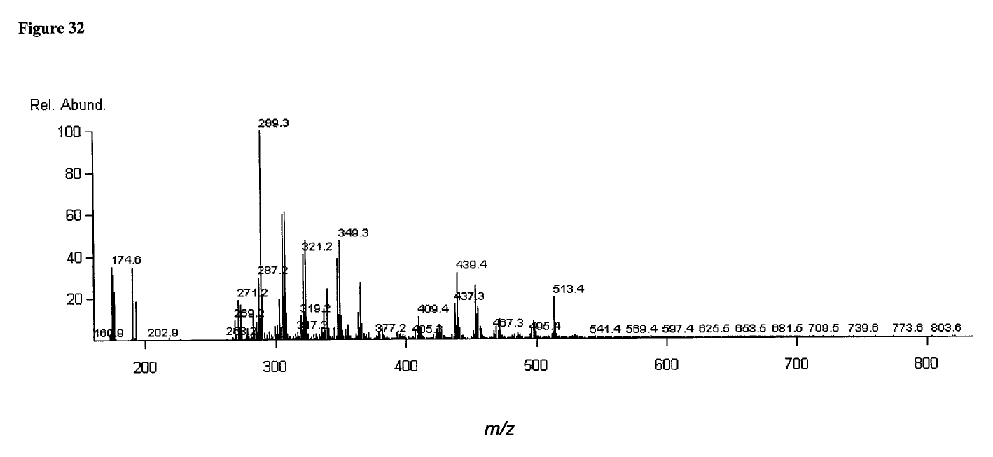

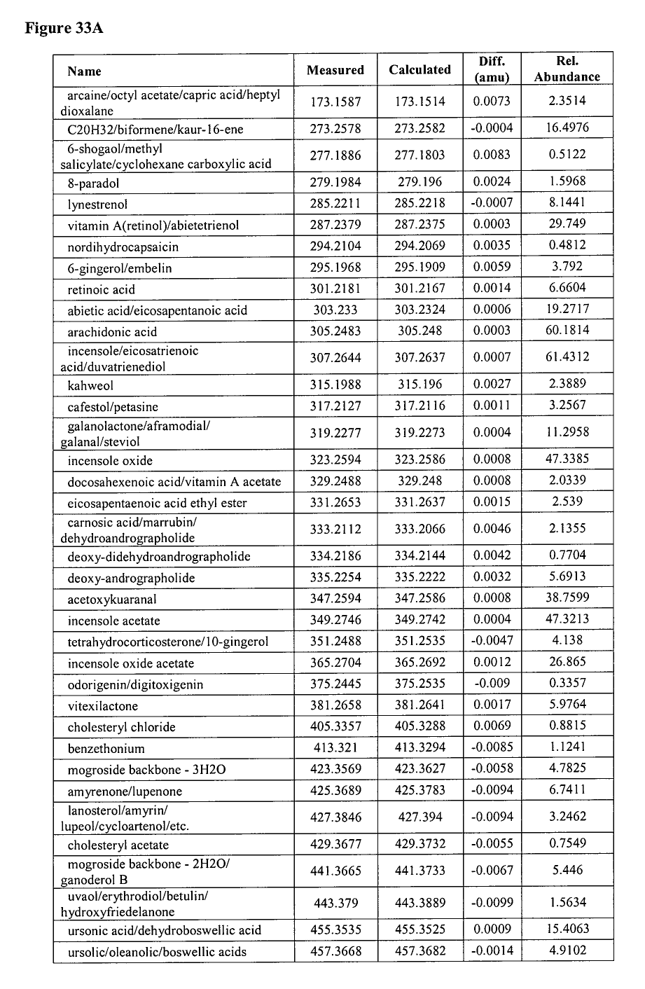

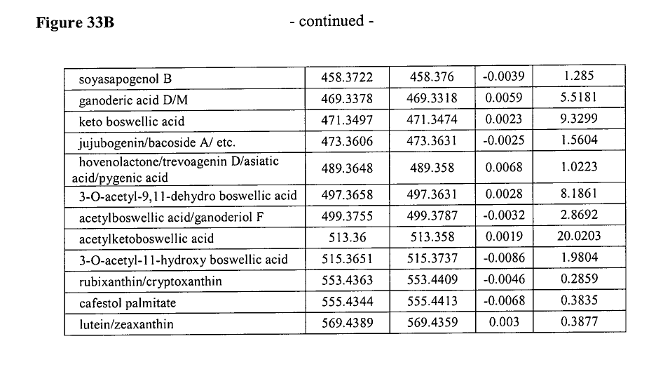

F. 60% ETHANOL EXTRACT (400 C) OF BOSWELLIA CARTERI. Many of the previously discussed compounds are present in the extract including biformene, vitamin A, arachidonic acid, incensole (and incensole derivatives), acetyloxykauranal, norhydrocapsaicin, gingerols, gymnemic acids, and ganoderols. Many xanthin derived compounds have been identified between m/z [M+H]+=565.4 and 731.5. The boswellic acids identified in the extract include KBA, ADHBA, ABA, AKBA, and AHBA. See FIGS. 32 and 33.

G. 80% ETHANOL EXTRACT OF BOSWELLIA CARTERI AT 40° C. Many of the previously discussed compounds are present in the extract including biformene, vitamin A, arachidonic acid, incensole (and incensole derivatives), acetyloxykauranal, norhydrocapsaicin, gingerols, and ganoderols. Many xanthin derived compounds are present between m/z [M+H]+=553.4 and 569.4. The boswellic acids identified in the extract include BA, DHBA, KBA, ADHBA, ABA, AKBA, and AHBA. See FIGS. 32 and 33.

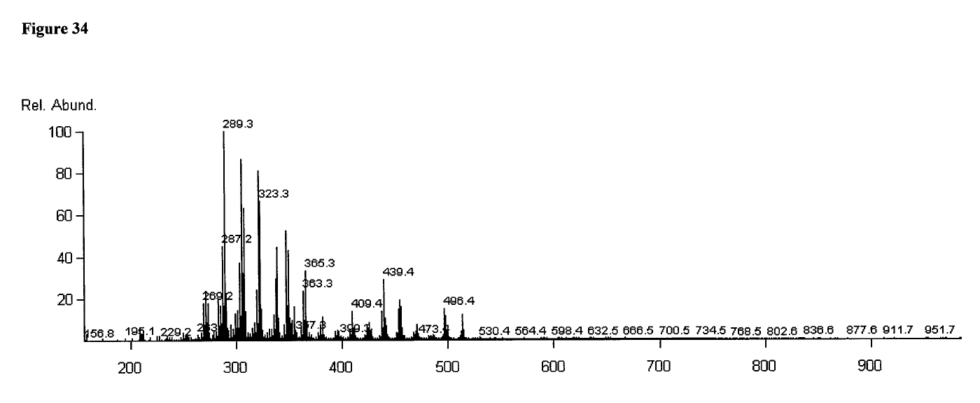

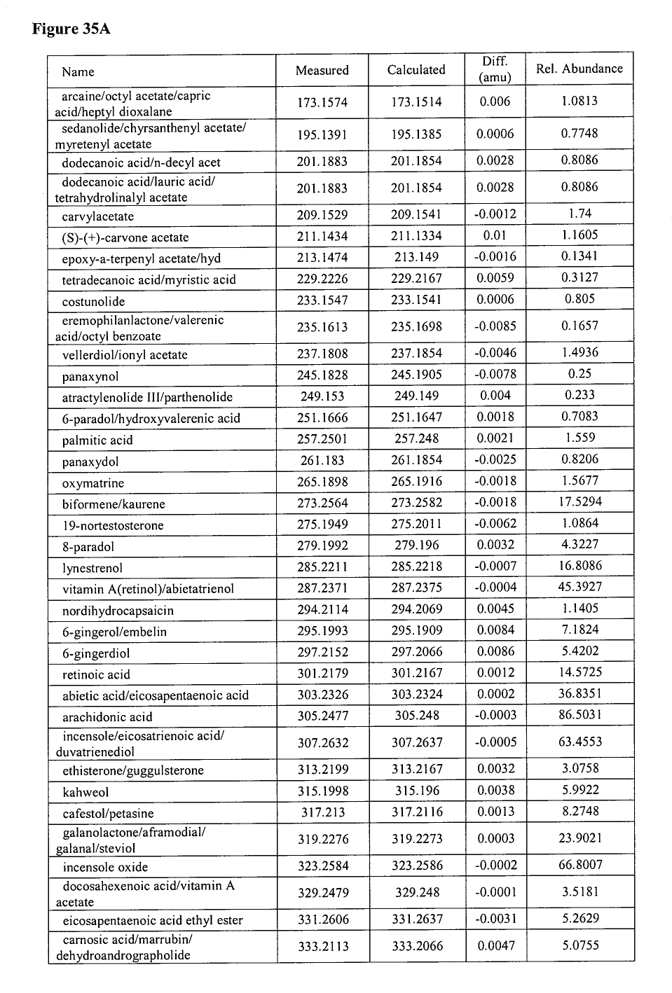

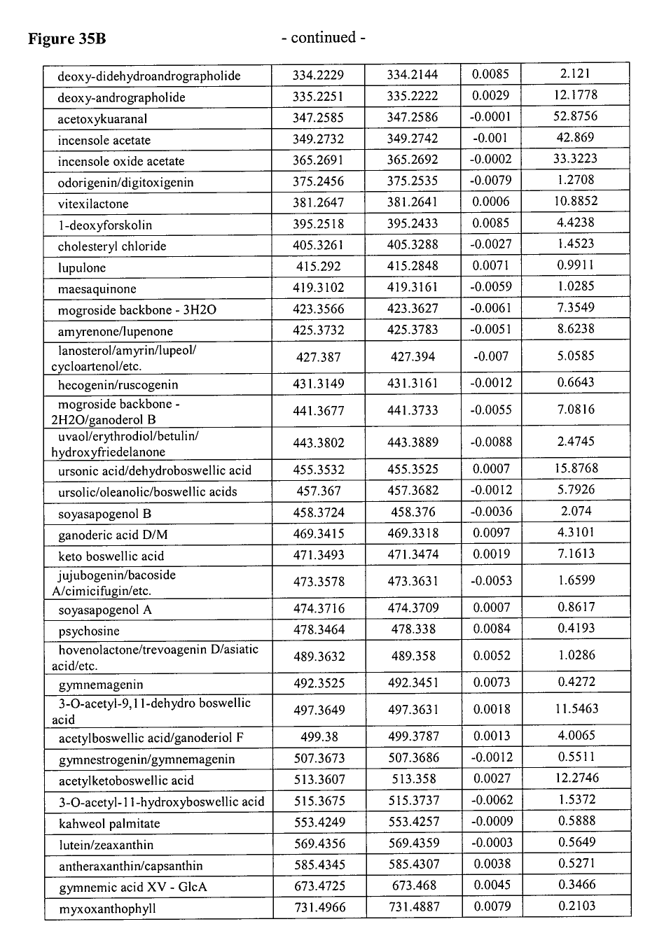

H. 100% ETHANOL EXTRACT OF BOSWELLIA CARTERI AT 40° C. Many of the previously discussed compounds are present in the extract including biformene, vitamin A, arachidonic acid, incensole (and incensole derivatives), acetyloxykauranal, norhydrocapsaicin, gingerols, ganoderols, ganoderic acids, and gymnemic acids. Many xanthin derived compounds are present between m/z [M+H]+=569.4 and 731.5. The boswellic acids identified in the extract include DHBA, BA, KBA, ADHBA, ABA, AKBA, and AHBA. See FIGS. 34 and 35.

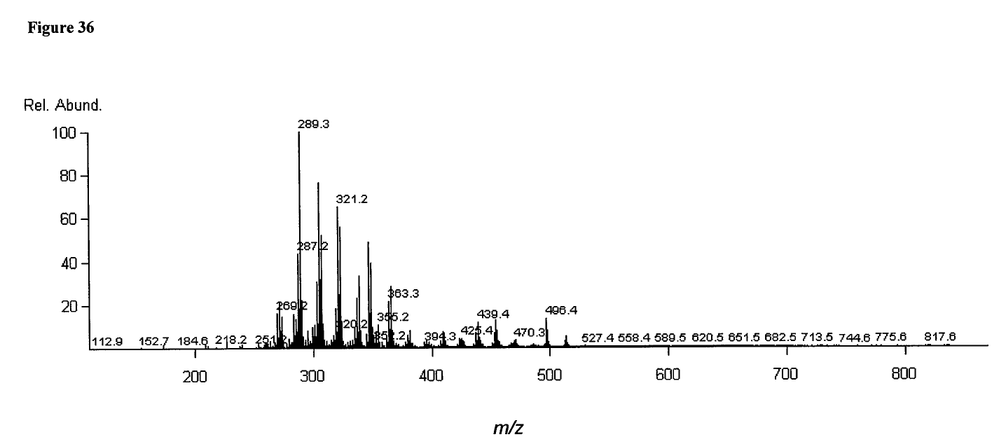

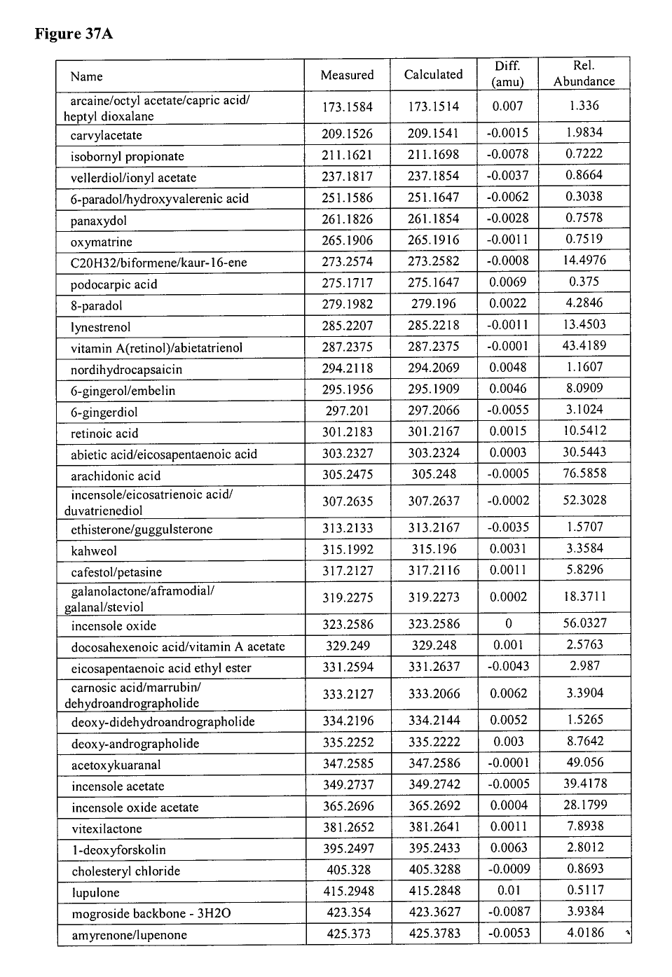

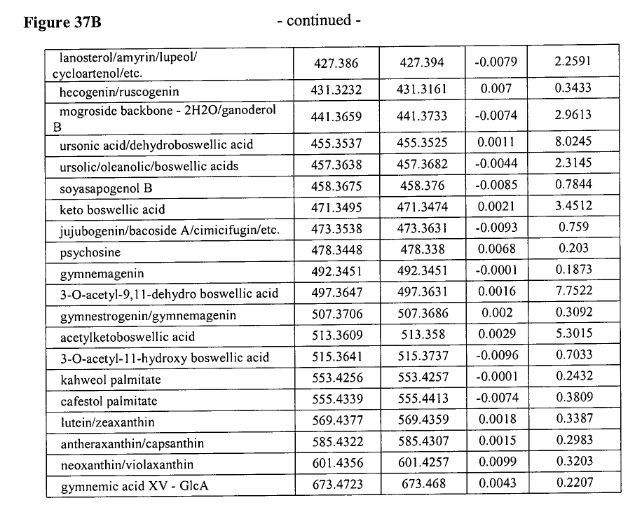

I. 80% ETHANOL EXTRACT OF BOSWELLIA CARTERI AT 60° C. Many of the previously discussed compounds are present in the extract including biformene, vitamin A, arachidonic acid, incensole (and incensole derivatives), acetyloxykauranal, norhydrocapsaicin, gingerols, ganoderols, ganoderic acids, and gymnemic acids. Many xanthin derived compounds are present between m/z [M+H]+=569.4 and 601.4. The boswellic acids identified in the extract include DHBA, BA, KBA, ADHBA, ABA, AKBA, and AHBA. See FIGS. 36 and 37.

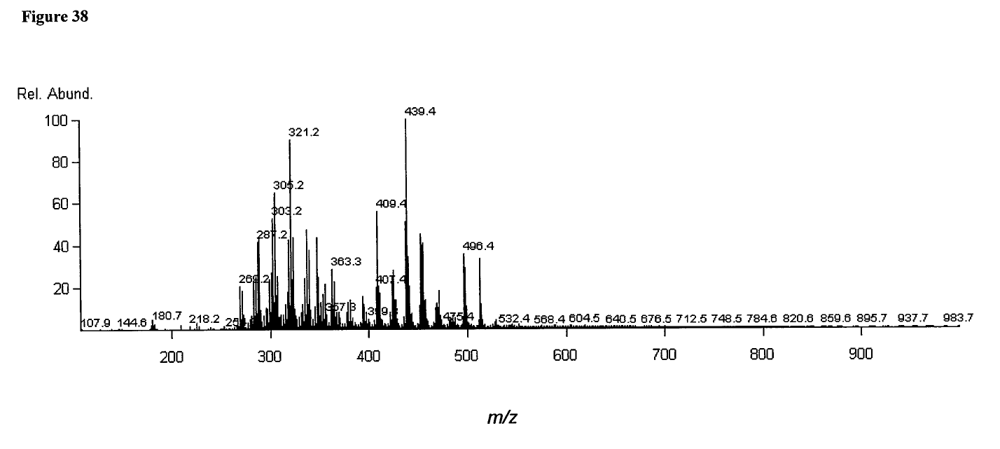

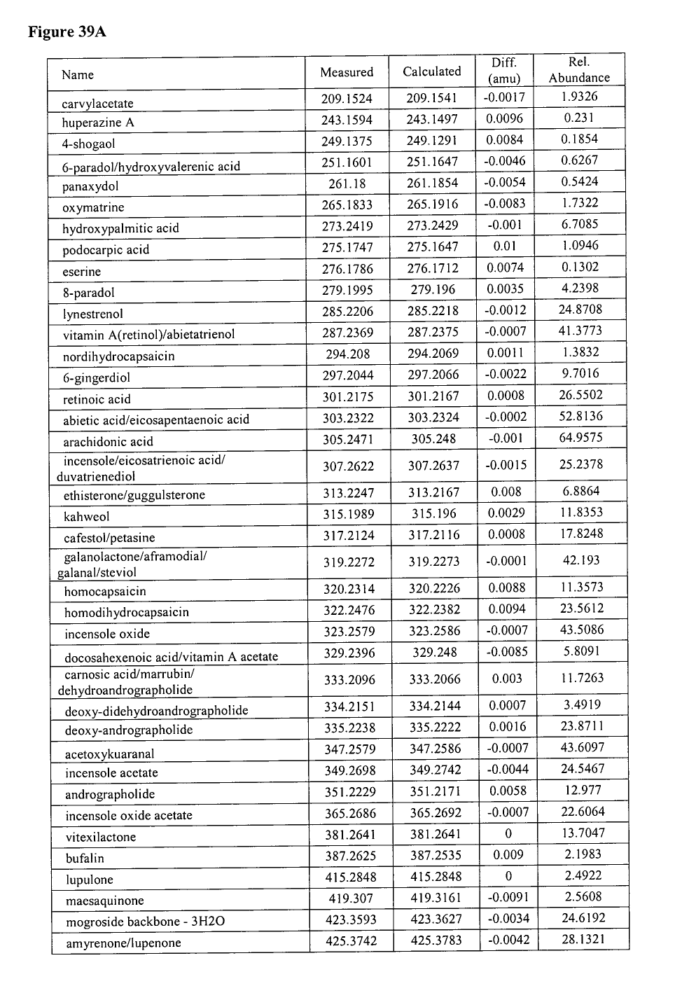

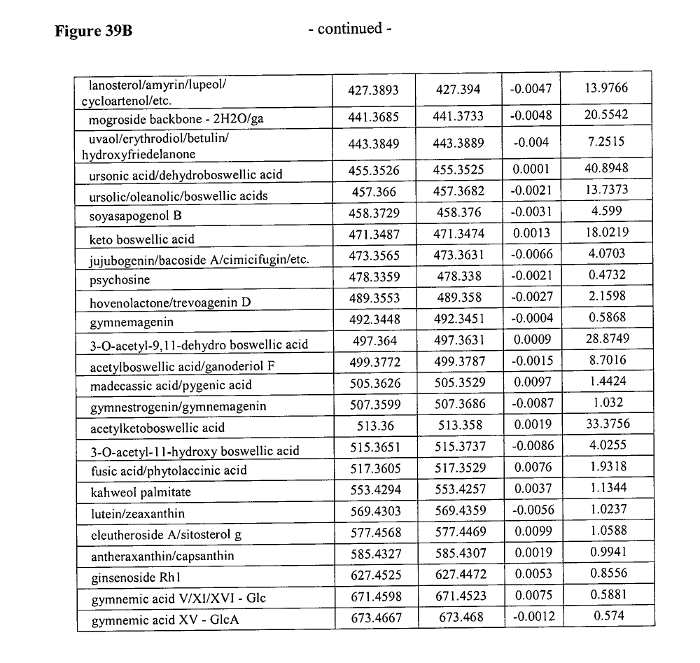

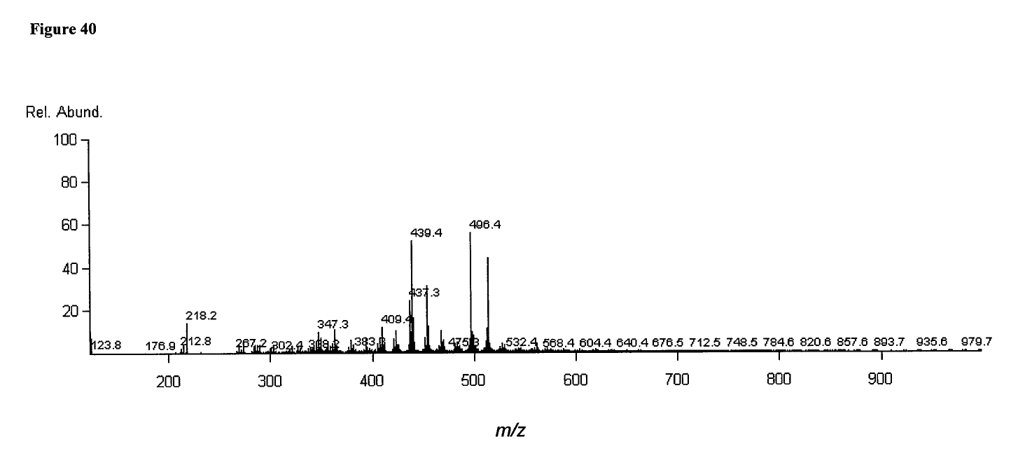

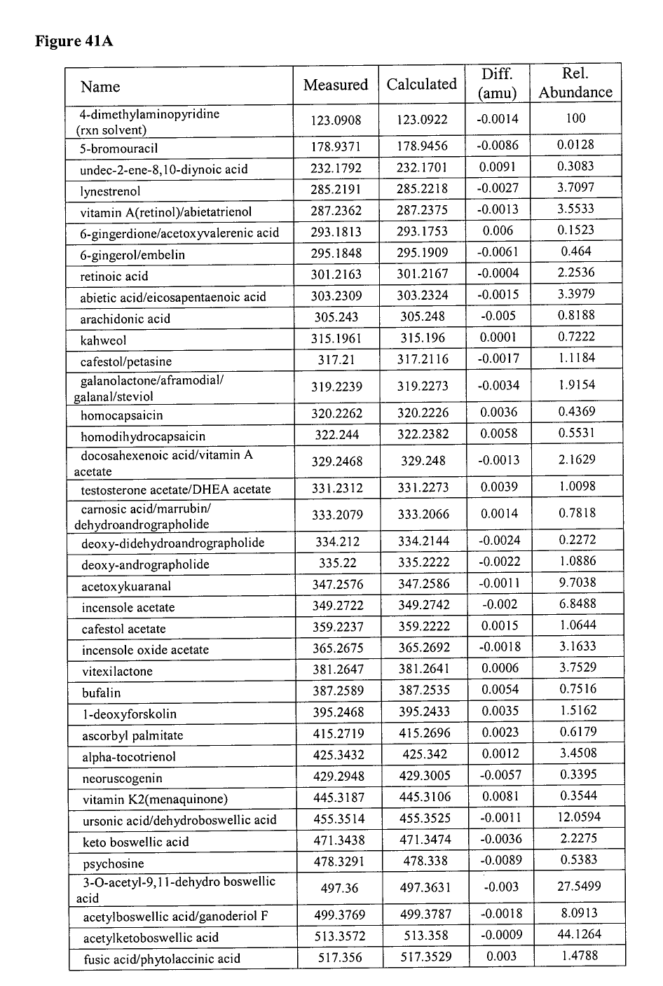

J. 100% ETHANOL EXTRACT OF BOSWELLIA CARTERI AT 60° C. Many of the previously discussed compounds are present in the including biformene, vitamin A, arachidonic acid, incensole (and incensole derivatives), acetyloxykauranal, norhydrocapsaicin, gingerols, ganoderols, ganoderic acids, shogaols, and gymnemic acids. Some xanthin derived compounds are present between m/z [M+H]+=569.4 and 585.4. The boswellic acids identified in the extract include DHBA, BA, KBA, ADHBA, ABA, AKBA, and AHBA. See FIGS. 38 and 39.

### Example 2

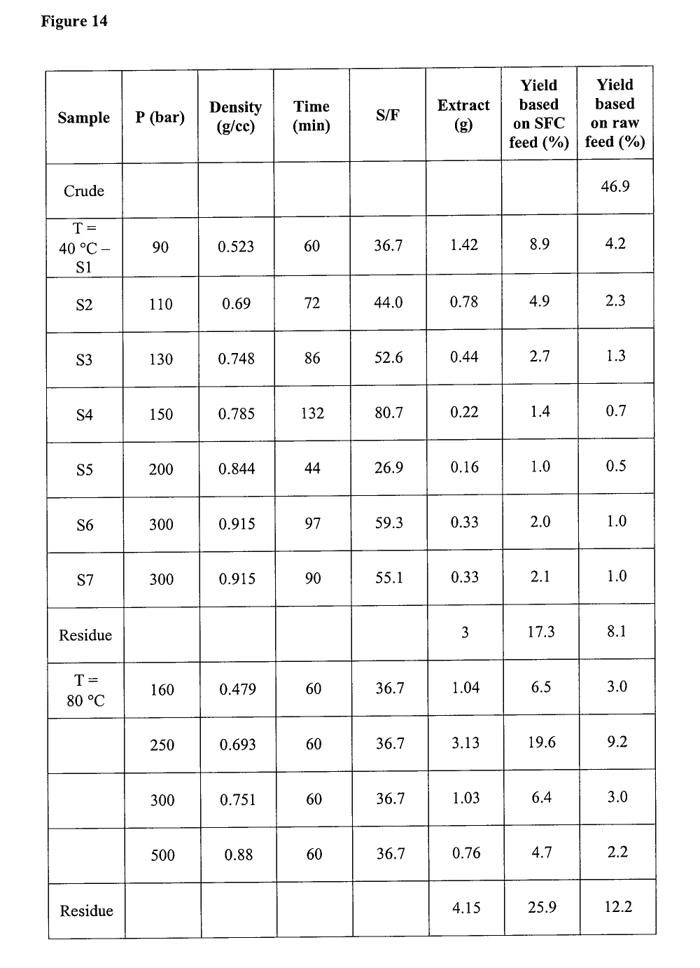

MULTI-STAGE SCCEP OF BOSWELLIA SPECIES LIPID SOLUBLE COMPOUNDS. Multi-stage SCCEP extraction/fractionation was performed using a SFT 250 (Supercritical Fluid Technologies, Inc., Newark, Del., USA). In typical multi-stage SCCEP extractions, silica gel 1 was soaked in ethanol for 12 hours. A 40 mL samples of the concentrated crude ethanol leaching extract solution was mixed with 30 gm of the silica gel. The silica gel-ethanol extract mixture was then loaded into an extraction vessel with an internal volume of 100 mL. The extraction solution was collected in a 40 mL collector vessel connected to the exit of the extraction vessel. The flow rate of CO₂ was set at 5 SLPM (10 g/min). The temperature was fixed at 40° C. The first extraction stage (S1) was performed at a pressure of 90 bar. This extraction stage as well as all subsequent stages was carried out until no further extract was measured at the collection port. The second extraction stage (S2) was performed at a pressure of 110 bar. The third extraction step (S3) was performed at a pressure of 130 bar. A fourth extraction stage (S4) was performed at a pressure of 150 bar. A fifth extraction stage (S5) performed at a pressure of 200 bar. A sixth extraction stage (S6) was performed at a pressure of 300 bar. A seventh extraction stage (S7) was performed at a pressure of 300 bar with ethyl acetate as a co-solvent. The residue in the extraction vessel was collected as the boswellic acid fraction. Samples from these fractions were dissolved in dichloromethane at a concentration of 400 ppm for GC-MS analysis. The analytical results are shown in FIG. 14.

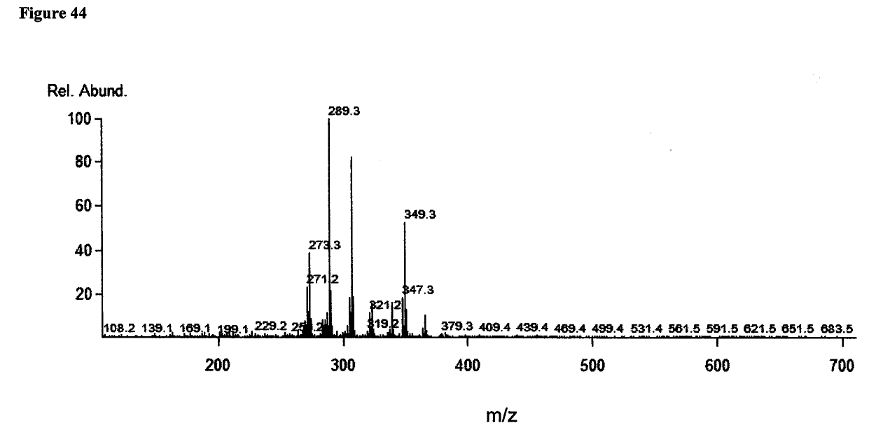

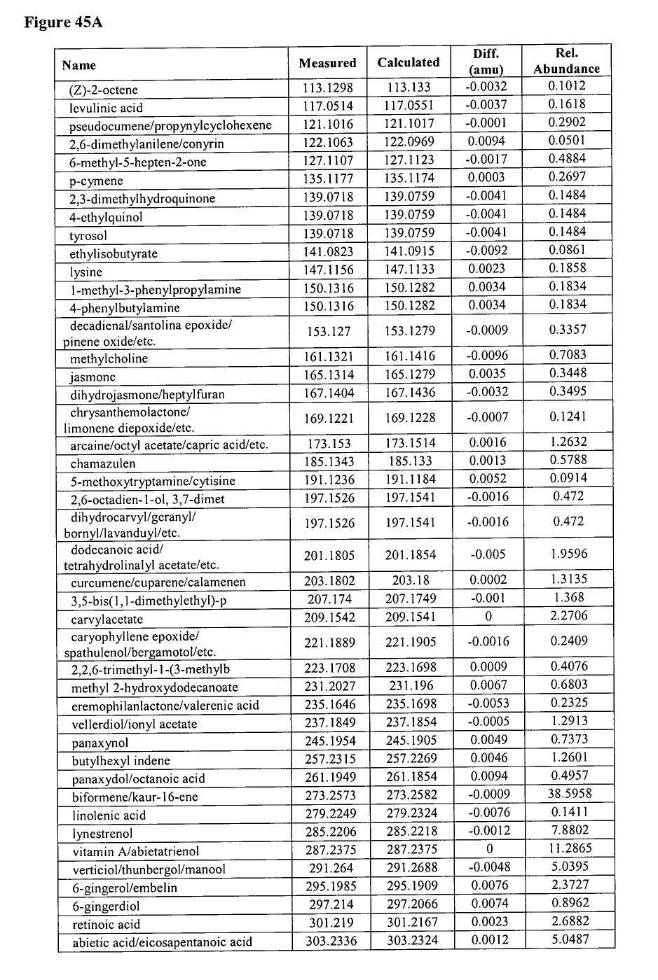

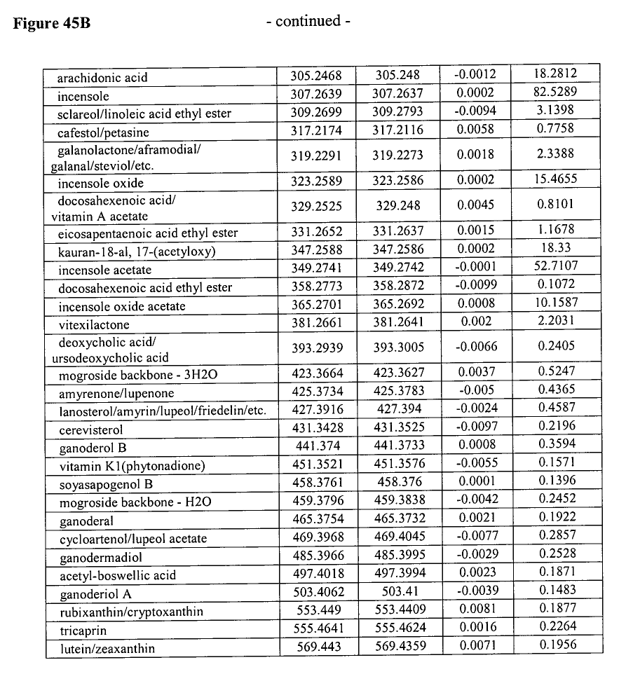

S1 EXTRACTION; T=40° C., STAGE 1, P=90 BAR. Many lipophilic chemicals have been identified in the first stage extraction of boswellia species. Acetyl-boswellic acid (ABA; m/z=497.3994) is identified at 0.2% relative abundance. Gingerols, phytosterols, xanthins, terpenoids, terpenols, ganoderols, vitamins, amino acids and fatty acids are also identified in the S1 extraction. See FIGS. 44 and 45.

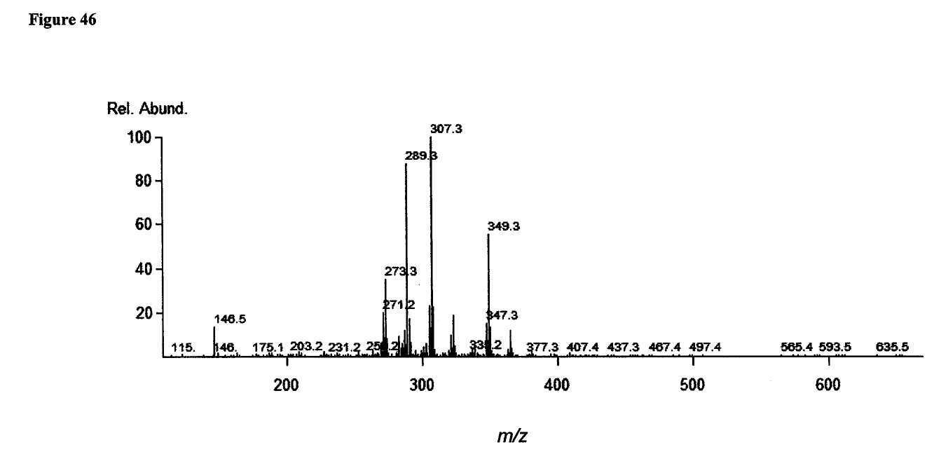

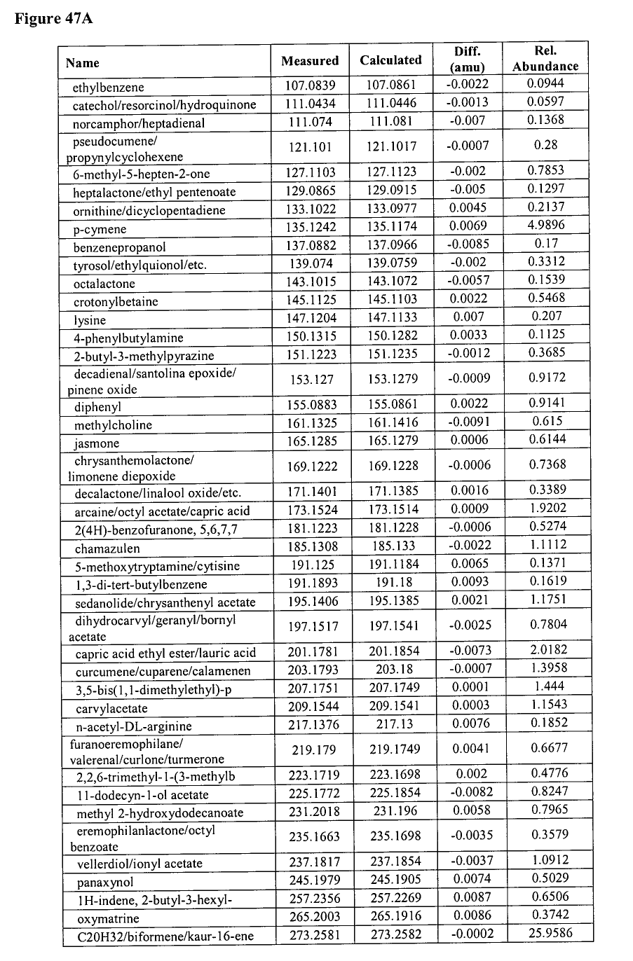

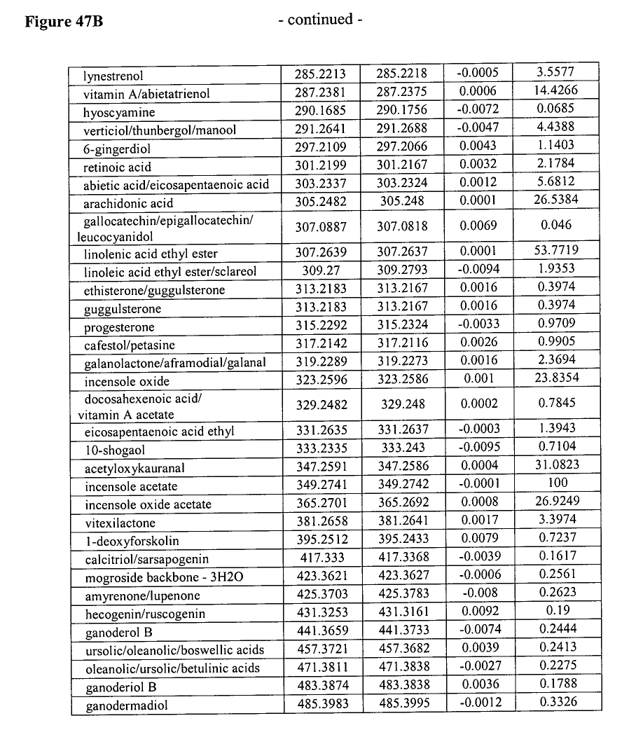

S2 EXTRACTION; T=40° C., STAGE 2, P=110 BAR. Many lipophilic chemicals have been identified in the 2nd stage extraction of boswellia species. Boswellic acid (BA; m/z=457.3682) is identified at 0.2% relative abundance. Gingerols, phytosterols, cholesterols, shogaols, gingerols, terpenoids, terpenols, ganoderols, vitamins, amino acids, flavonoids and fatty acids are identified in the S2 extraction. See FIGS. 46 and 47.

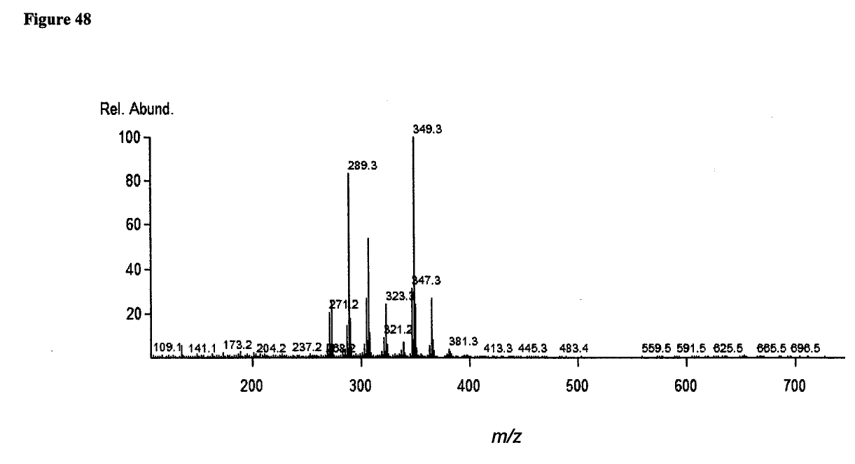

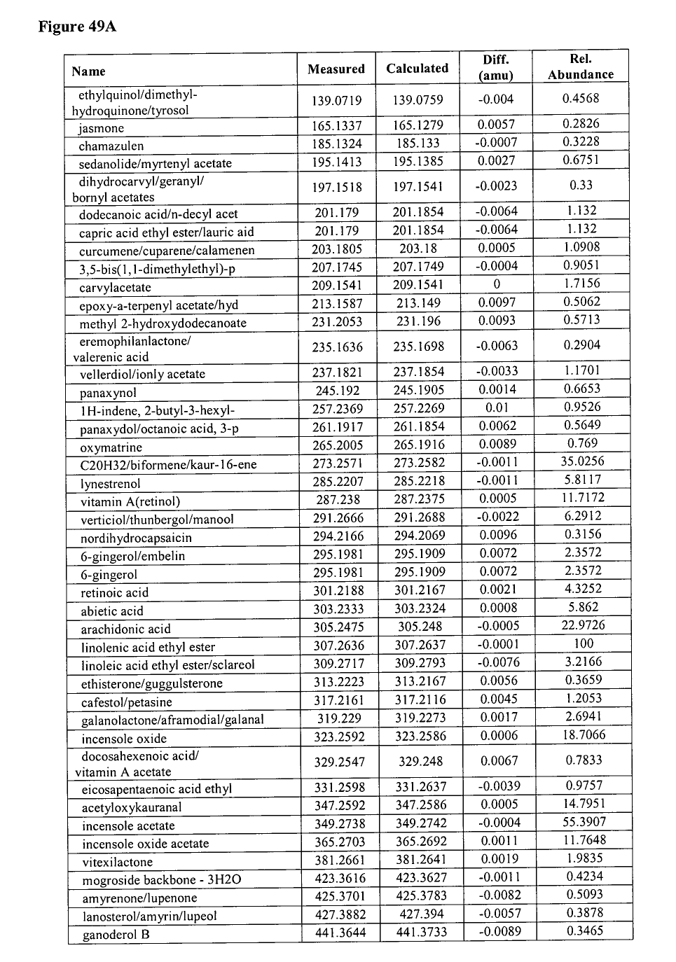

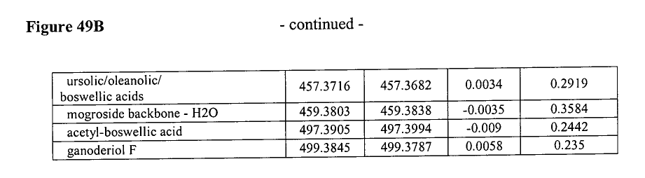

S3 EXTRACTION; T=40° C., stage 3, P=130 bar. Many lipophilic chemicals have been identified in the first stage extraction of boswellia species. ABA and BA are identified at 0.2 and 0.3% relative abundance, respectively. Gingerols, phytosterols, lactones, ganoderols, terpenoids, terpenols, vitamins, fatty acids, gingerols, phenols, and capsaicins are identified in the S3 extract. See FIGS. 48 and 49.

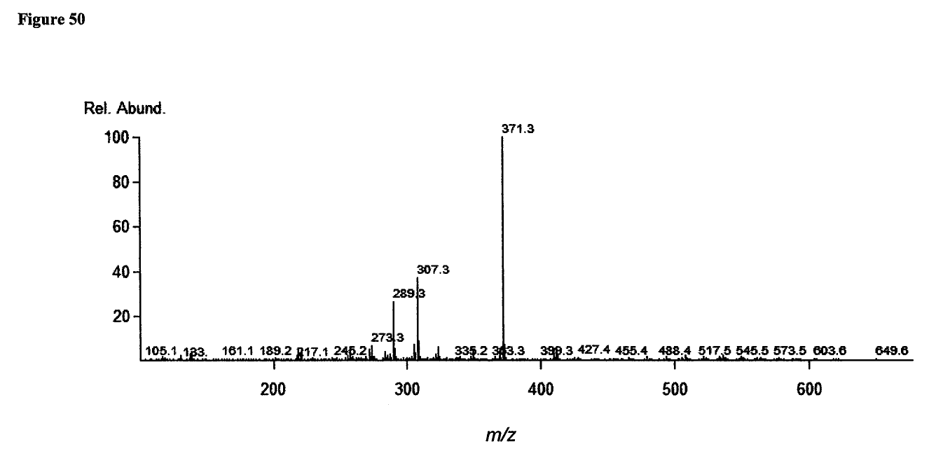

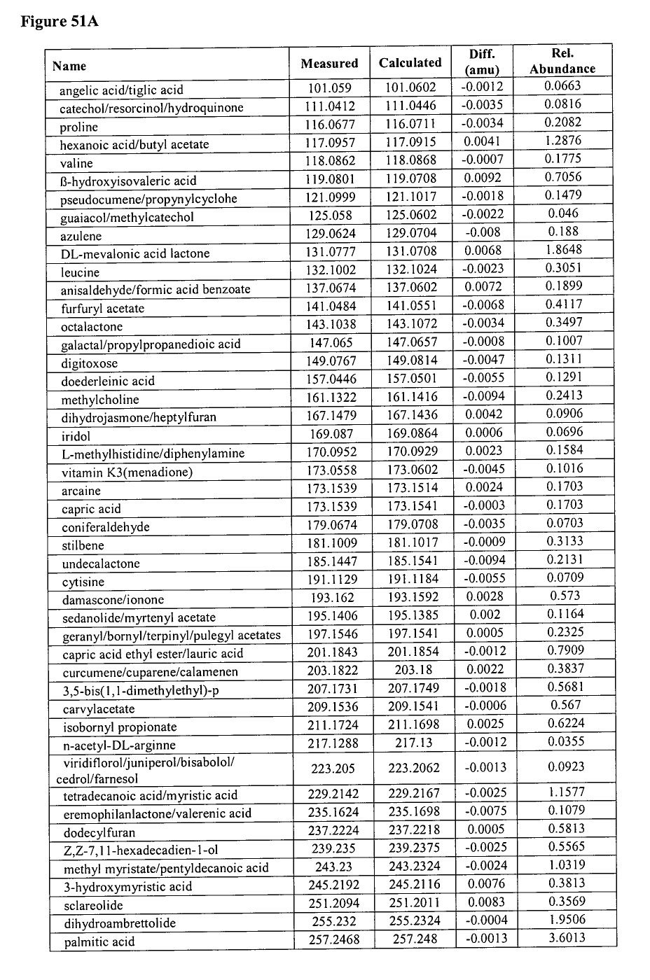

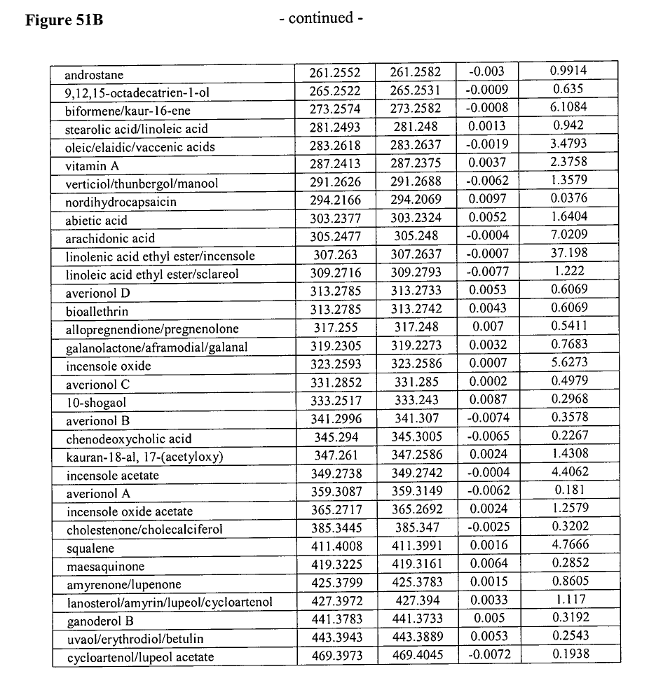

S4 EXTRACTION; T=40° C., STAGE 4, P=150 BAR. No boswellic acids or acetylated boswellic acid have been identified by DART-MS in the S4 extract. Amino acids, vitamins, fatty acids, capsaicins, shogaols, flavonoids (particularly the previously described Averionols at m/z=359.3, 341.3, 331.3, and 313.3), terpenoids, and phytosterols are identified in the S4 extract. See FIGS. 50 and 51.

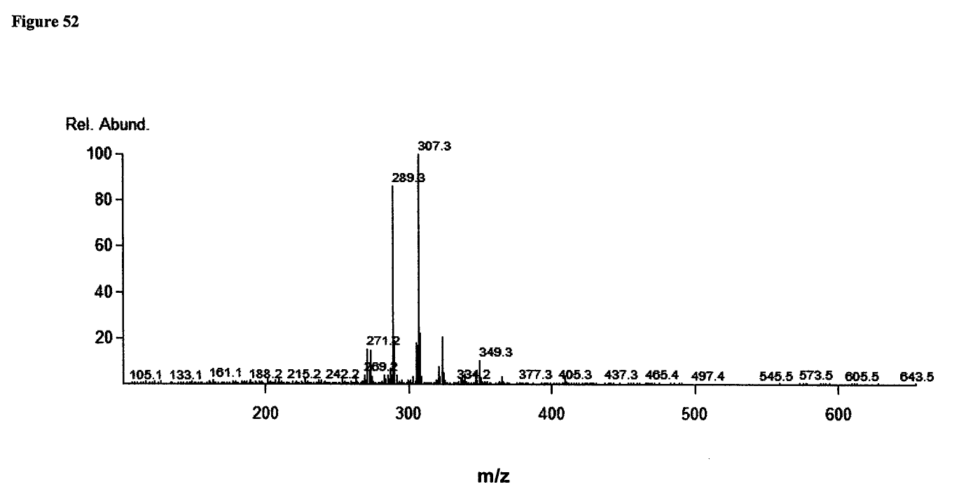

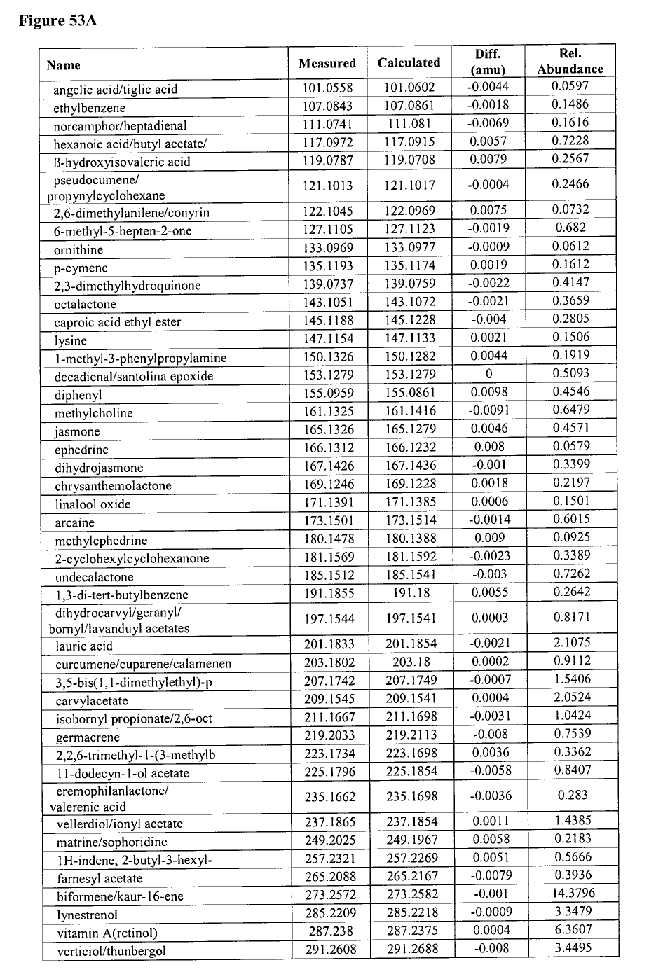

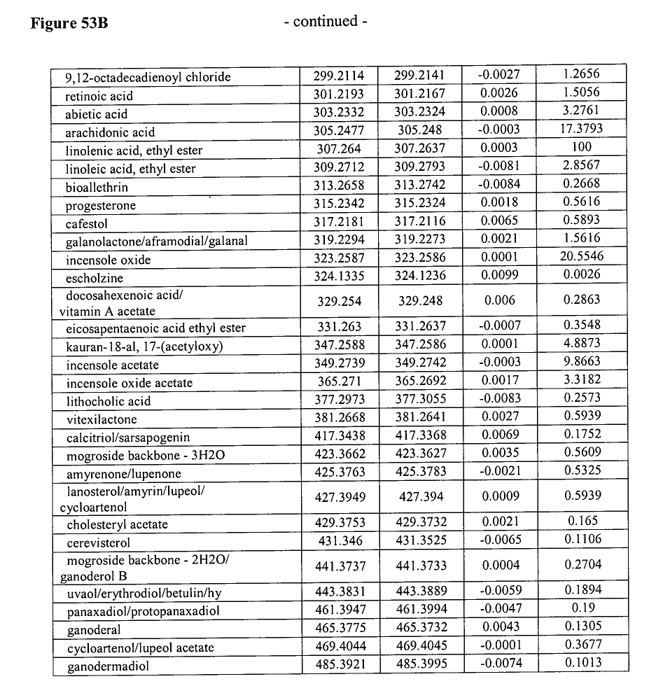

S5 EXTRACTION; T=40° C., STAGE 5, P=200 BAR. No boswellic acids or acetylated boswellic acid derivatives have been identified by DART-MS in the S5 extract. Amino acids, lactones, vitamins, fatty acids, alkaloids, terpenoids, phytosterols, phenols, terpenoids, and ganoderols are identified in the S5 extract. See FIGS. 52 and 53.

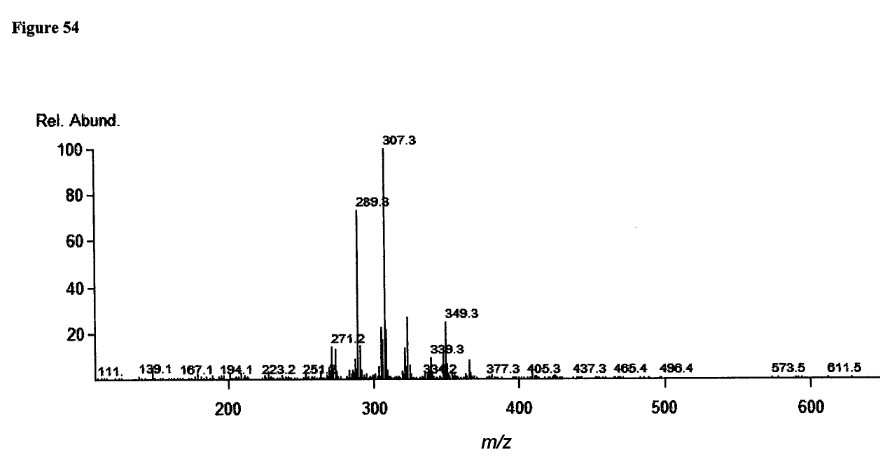

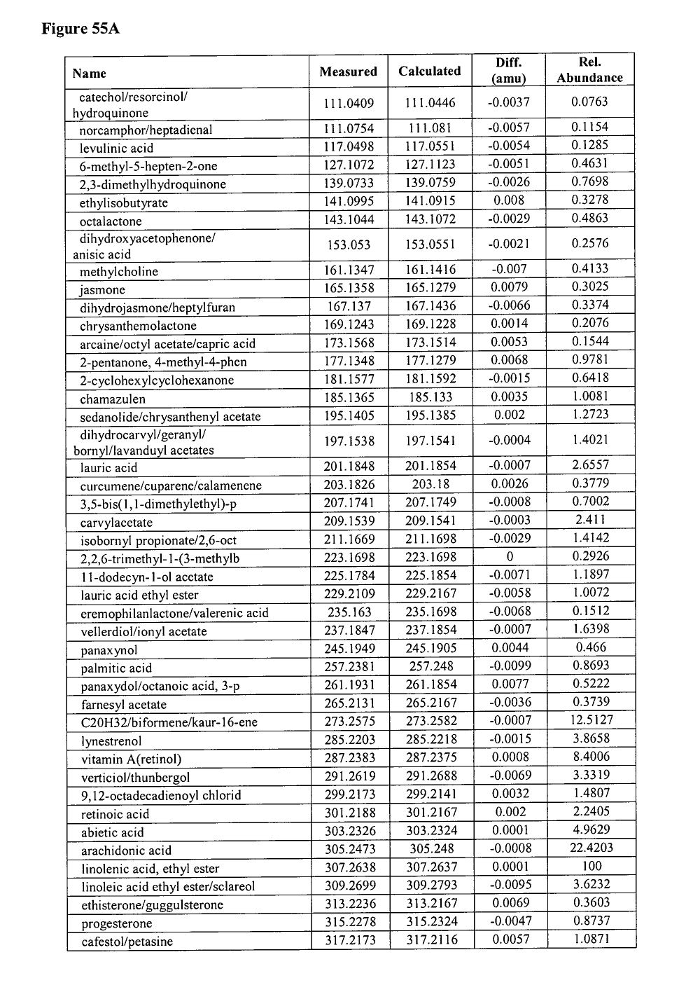

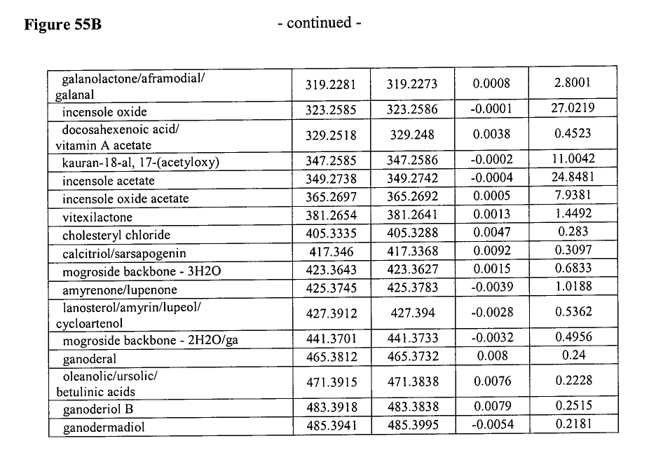

S6 EXTRACTION; T=40° C., STAGE 6, P=300 BAR. No boswellic acids or acetylated boswellic acid derivatives have been identified by DART-MS in the S6 extract. Flavonoids, terpenoids, fatty acids, lactones, phenols, terpenoids, ganoderols, vitamins, alkaloids, phytosterols, and quinones are identified in the S6 extract. See FIGS. 54 and 55.

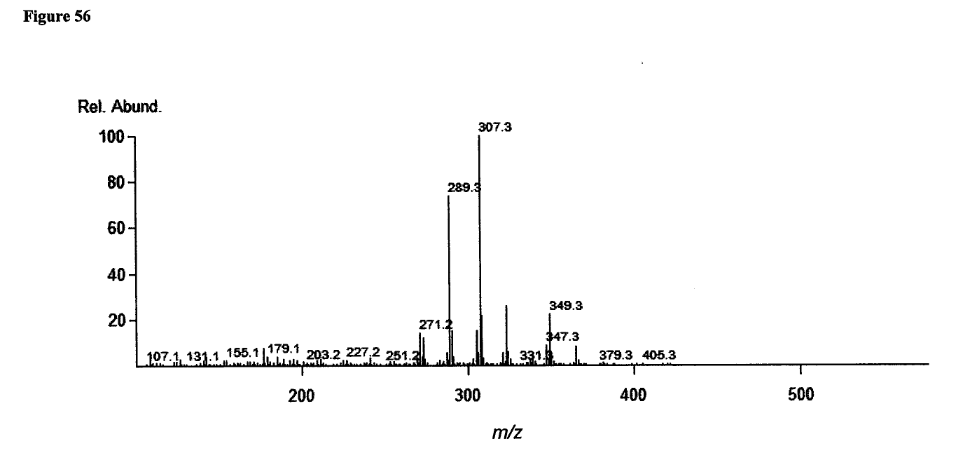

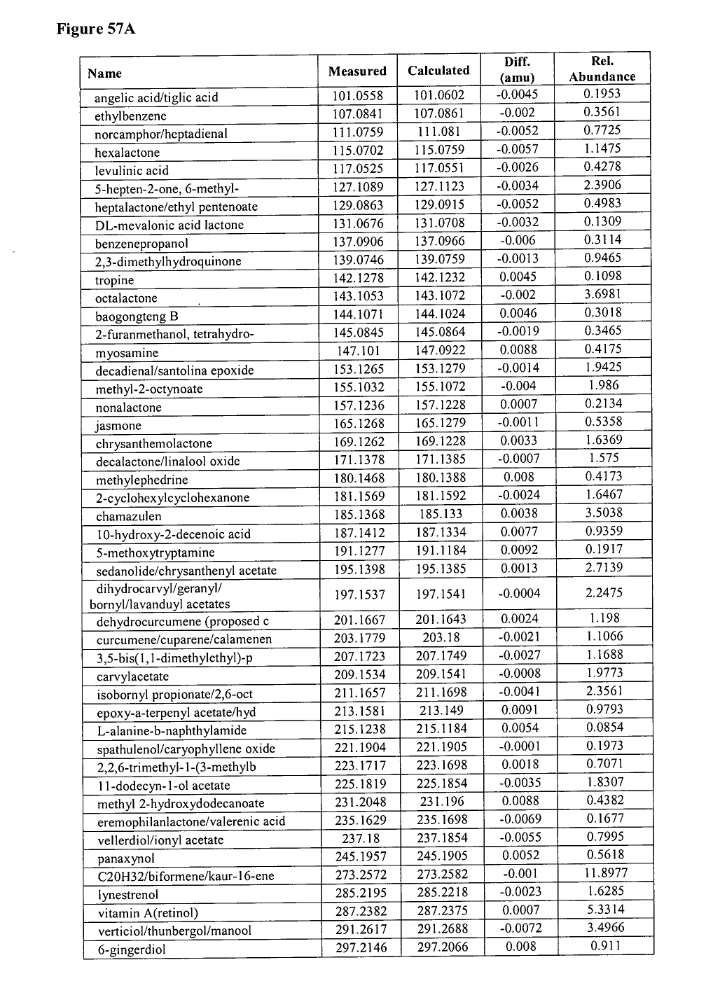

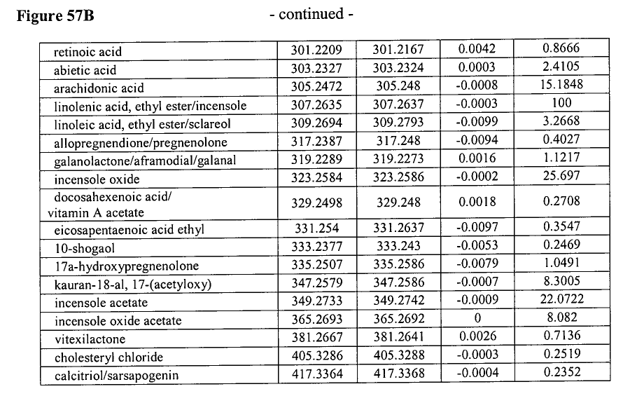

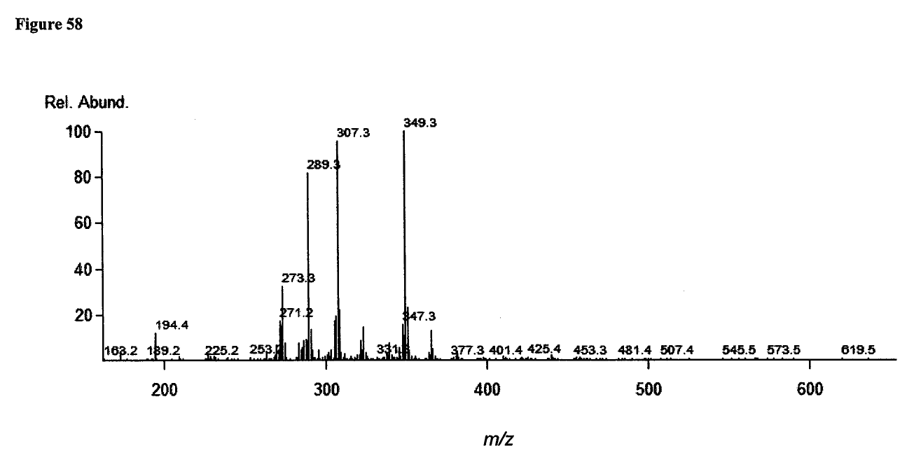

S7 EXTRACTION; T=40° C., STAGE 7, P=300 BAR WITH 3% ETHYL ACETATE. No boswellic acids or acetylated boswellic acid derivatives have been identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 56 and 57.

### Example 3

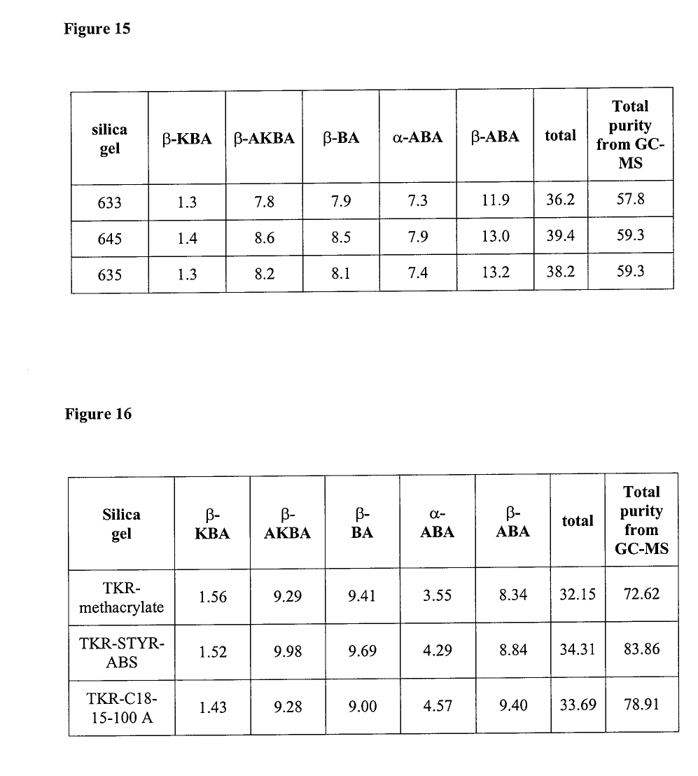

EXAMPLE OF STEP 2 MULTI-STAGE SCCEP OF BOSWELLIA SPECIES LIPID SOLUBLE EXTRACTION PROCESSES USING SILICA GEL AFFINITY ADSORBENTS WITH DIFFERING PARTICLE OR PORE SIZE. In typical experimental multi-stage SCCEP, 40 mL of Boswellia species concentrated ethanol leaching solution (400 mg/mL) were mixed with 30 gm of silica gel in a clean beaker (0.5 gm extract/gm silica gel) and then loaded into a 100 mL SFT250 extraction vessel with both ends packed with glass wool. The extraction unit temperature was set at 40° C. and the system was equilibrium in this temperature for 15 minutes, then CO₂ flow was initiated at a flow rate of 10-20 g/min. The pressure was changed sequentially at 90 bar, 150 bar and 200 bar. At each pressure, the system was allowed to run for at least 1 hour until no further extract was observed at the sample port. Samples at the end of each processing stage were collected and weighed for yield calculation. The residues generated after each silica gel processing were also analyzed by HPLC and the results are shown in FIG. 15.

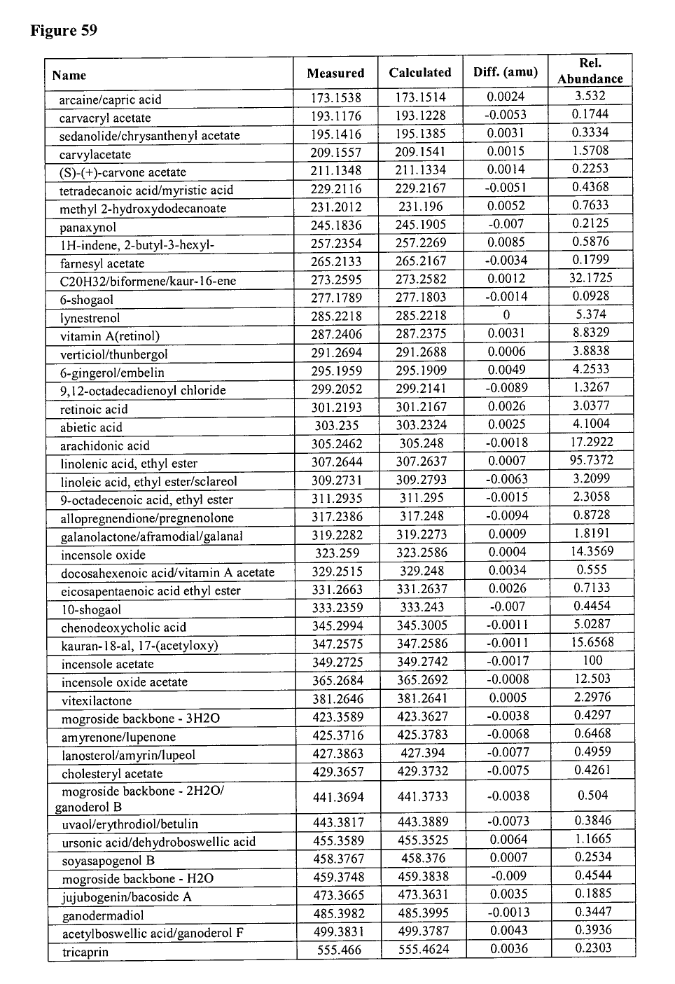

SILICA GEL 633 AT T=40° C., STAGE 1, P=100 BAR. Dehydroboswellic acid (DHBA) and ABA are identified in 1.2 and 0.4% relative abundance by DART-MS. Fatty acids, vitamins, gingerols, phytosterols, shogaols, lactones, terpenoids, ganoderols, and phenols are identified in the stage 1 extract. See FIGS. 58 and 59.

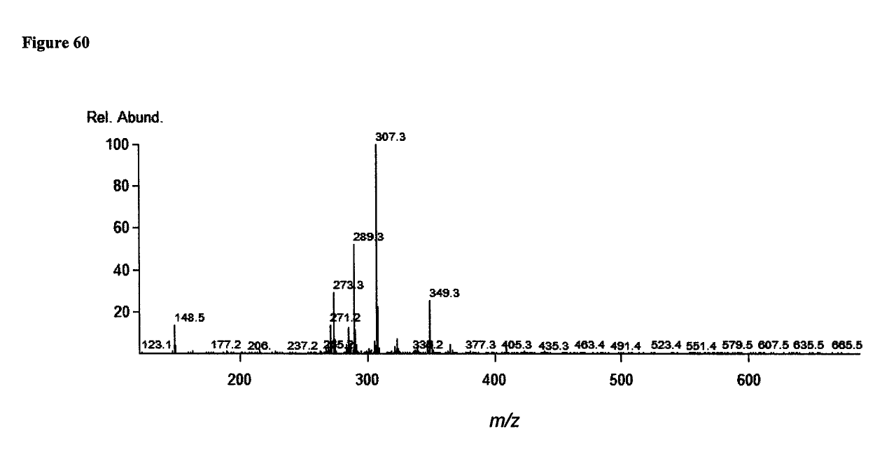

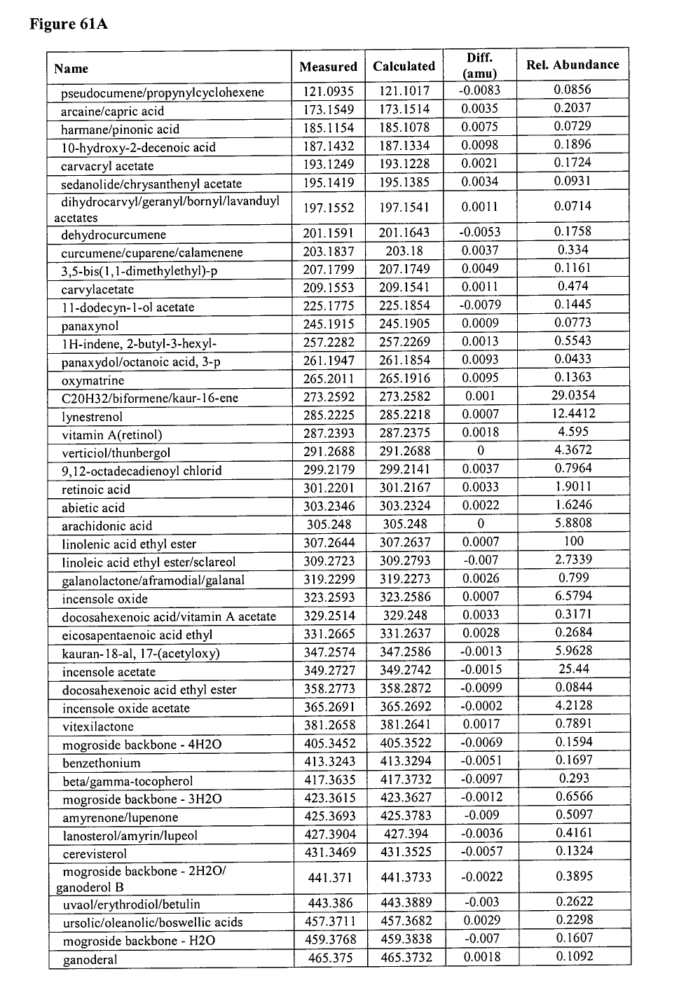

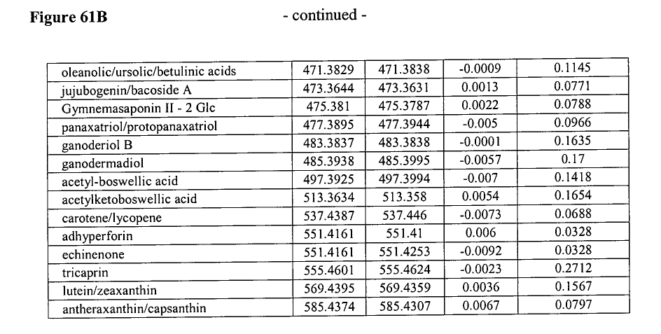

SILICA GEL 633 AT T=40° C., STAGE 2, P=150 BAR. BA, ABA, and acetylketoboswellic acid (AKBA) are have been identified by DART-MS in 0.2, 0.1, and 0.1% relative abundance. Fatty acids, hydrocarbons, terpenoids, phenols, ganoderols, phytosterols, vitamins, gingerols, alkaloids and xanthins are identified in the stage 2 extract. See FIGS. 60 and 61.

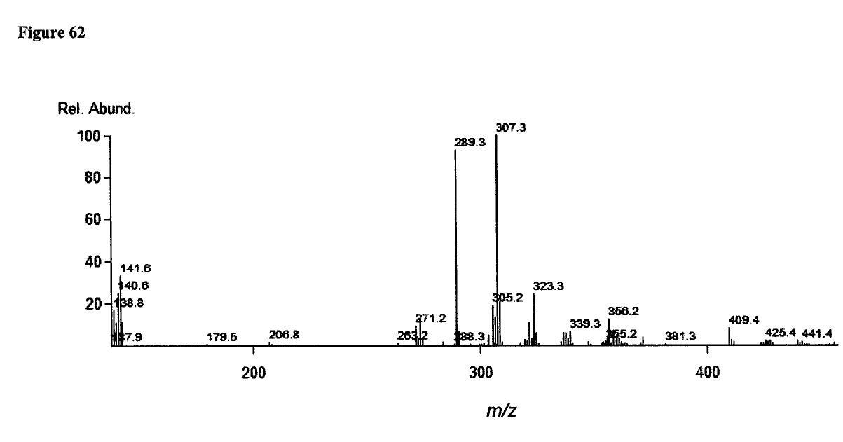

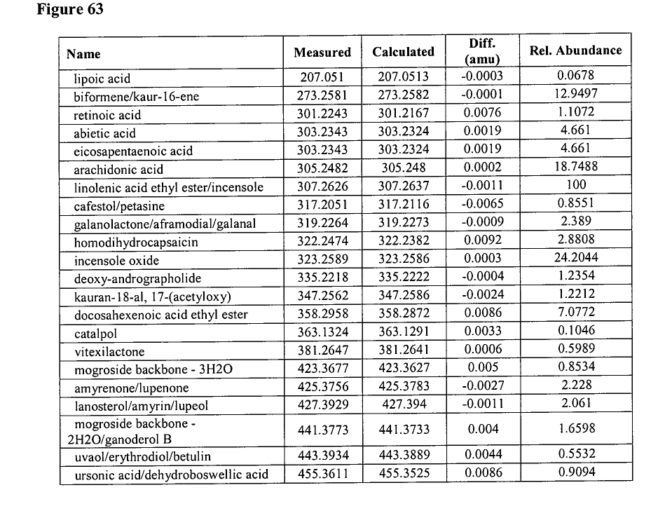

SILICA GEL 633 AT T=40° C., STAGE 3, P=200 BAR. DHBA at 0.9% relative abundance was the only boswellic acid or acetylated boswellic acid derivative identified in the stage 3 extract by DART-MS. The stage 3 extract was comprised mainly of fatty acids and phytosterols, although capsaicins and lactones are identified. See FIGS. 62 and 63.

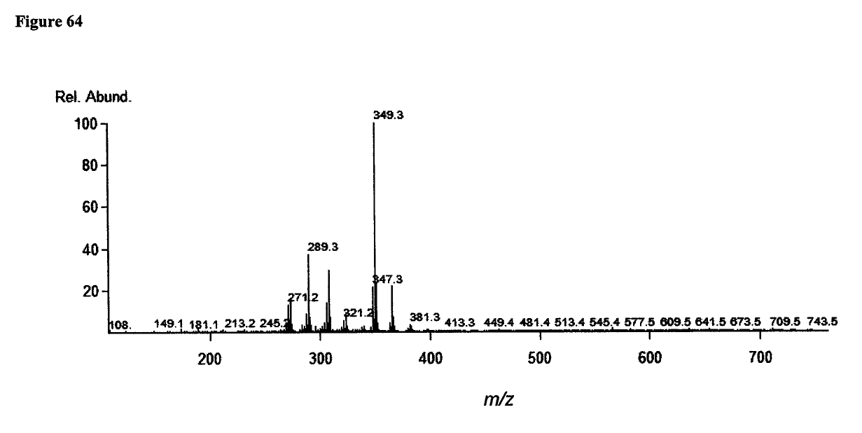

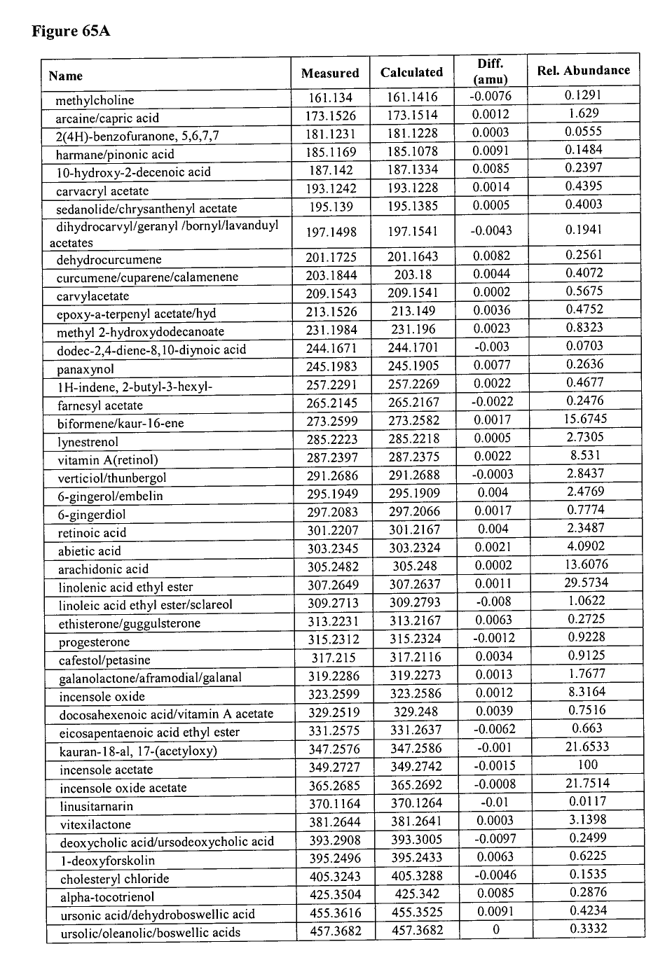

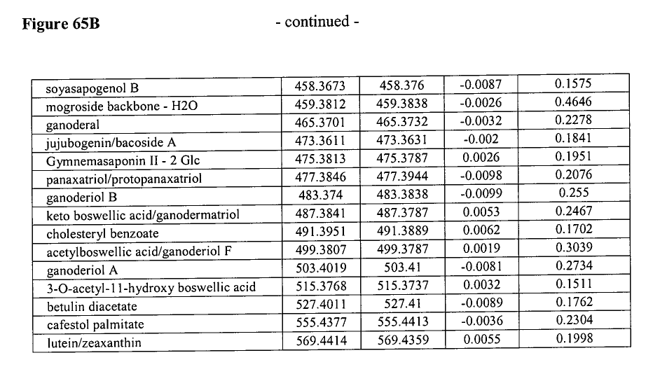

SILICA GEL 635 AT T=40° C., STAGE 1, P=100 BAR. DHBA, BA, ketoboswellic acid (KBA), ABA, acetyl hydroxyboswellic acid (AHBA) are present in the stage 1 extract at 0.4, 0.3, 0.2, 0.3, 0.1% relative abundance by DART-MS analysis. Fatty acids, hydrocarbons, phytosterols, gingerols, xanthins, vitamins and ganoderols are identified in the stage 3 extract. See FIGS. 64 and 65.

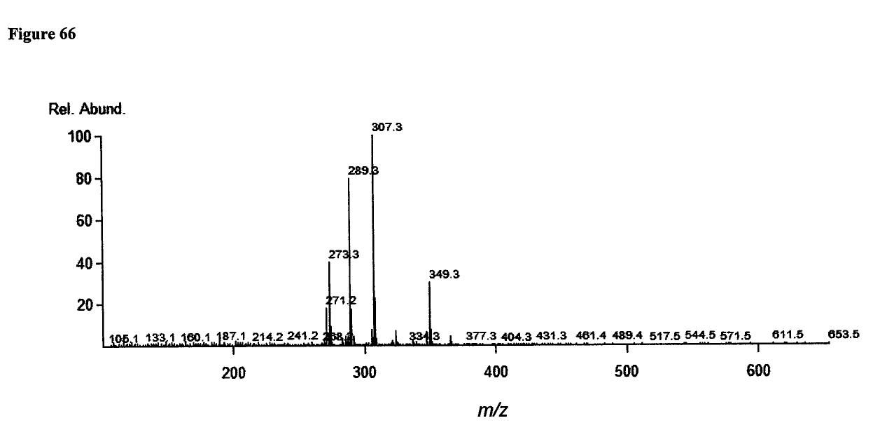

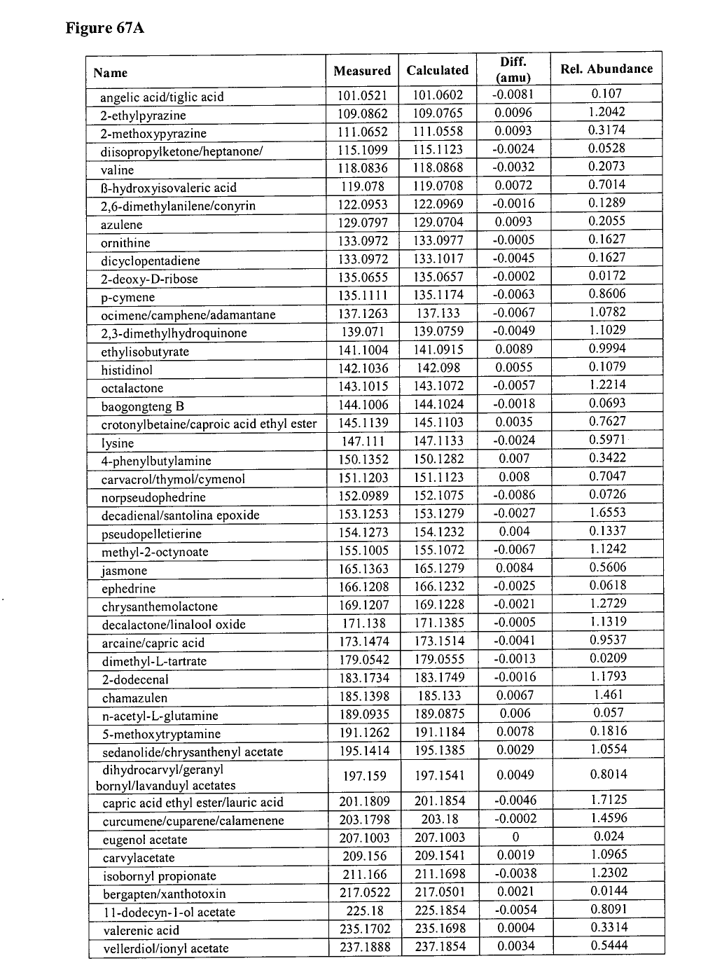

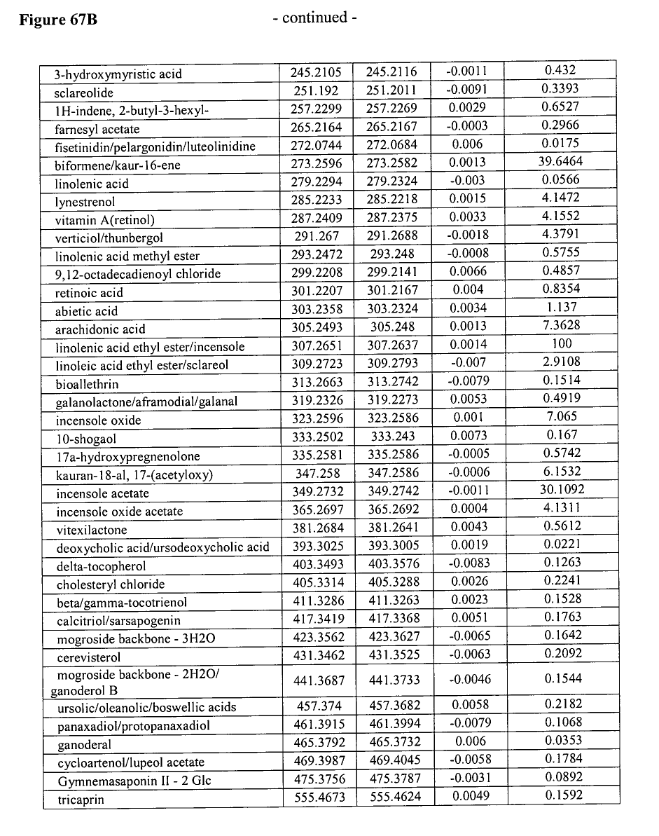

SILICA GEL 635 AT T=40° C., STAGE 2, P=150 BAR. BA is identified in 0.2% relative abundance in the stage 2 extract by DART-MS. Fatty acids, pyrazines, saccharides, alkaloids, hydrocarbons, amino acids, vitamins, lactones, terpenoids, phenols, ganoderols, and phytosterols are identified in the stage 2 extract. See FIGS. 66 and 67.

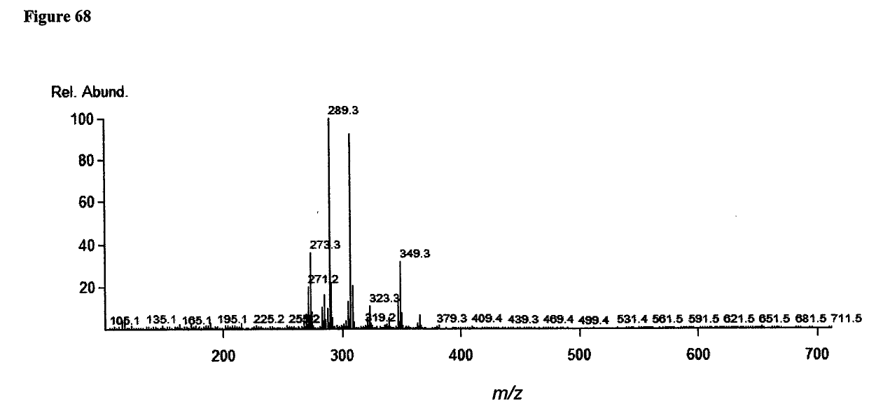

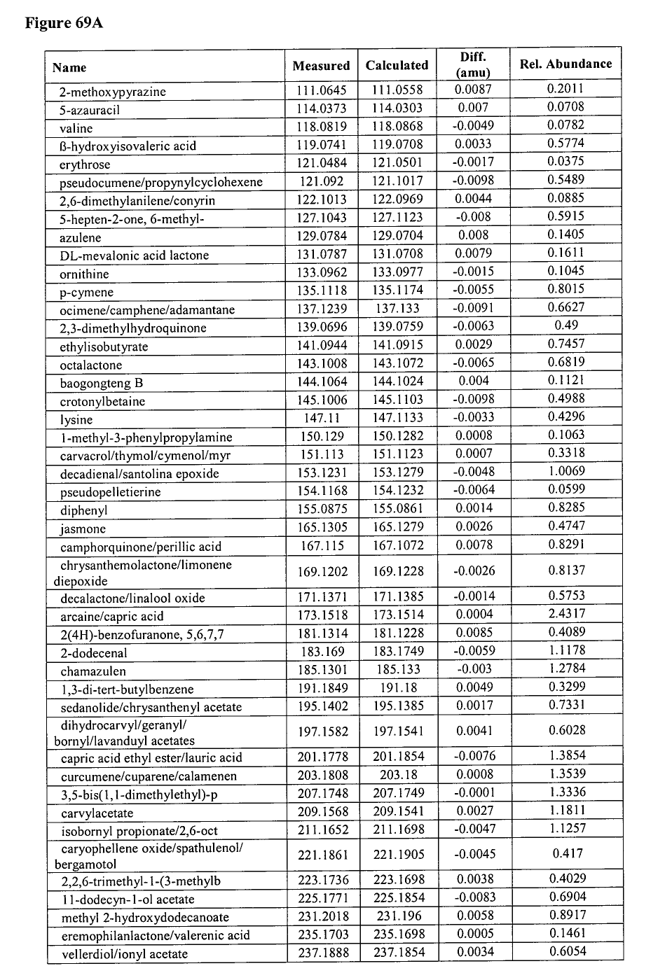

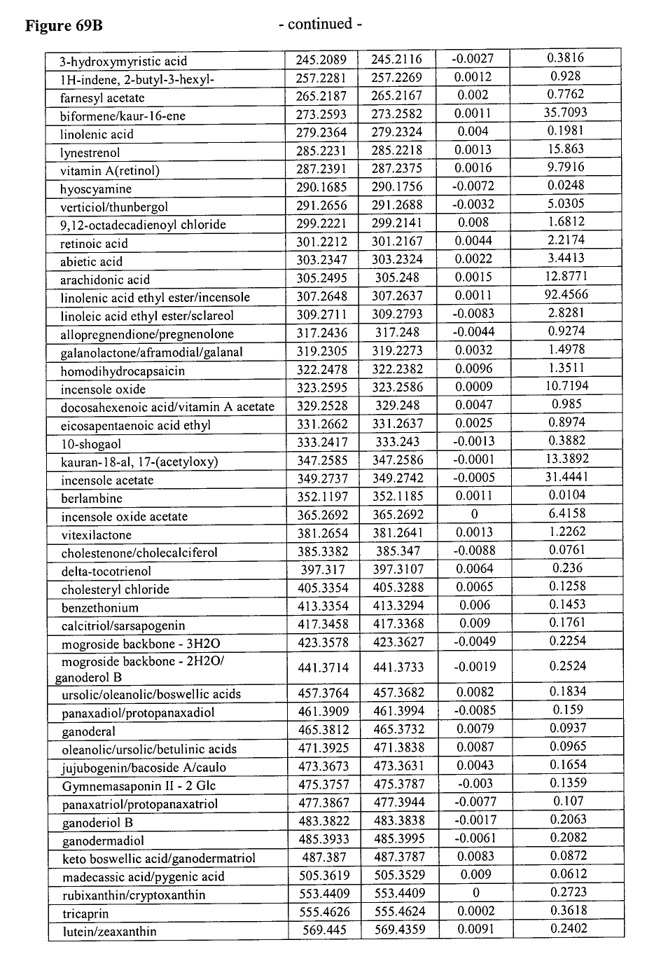

SILICA GEL 635 AT T=40° C., STAGE 3, P=200 BAR. BA and KBA are identified in the stage 3 extract at 0.2 and 0.1% relative abundance, respectively, by DART-MS. The stage 3 extract also contains many fatty acids, phytosterols, ganoderols, vitamins, alkaloids, pyrazines, gingerols, terpenoids, terpenols, vitamins, and lactones. See FIGS. 68 and 69.

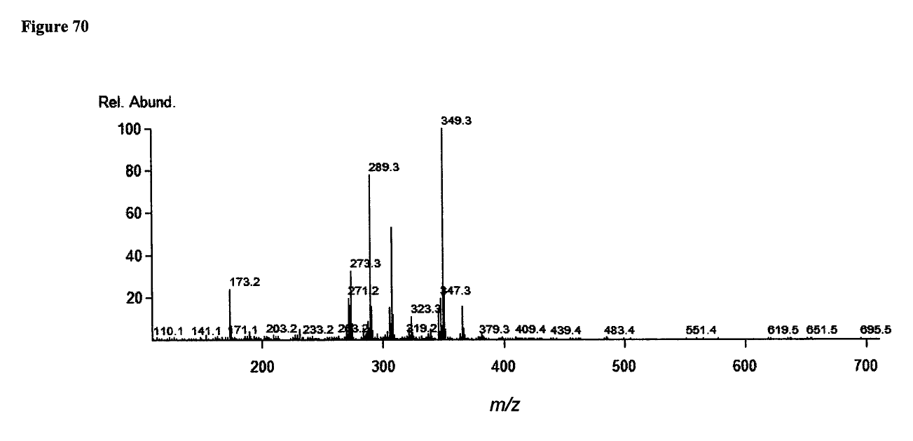

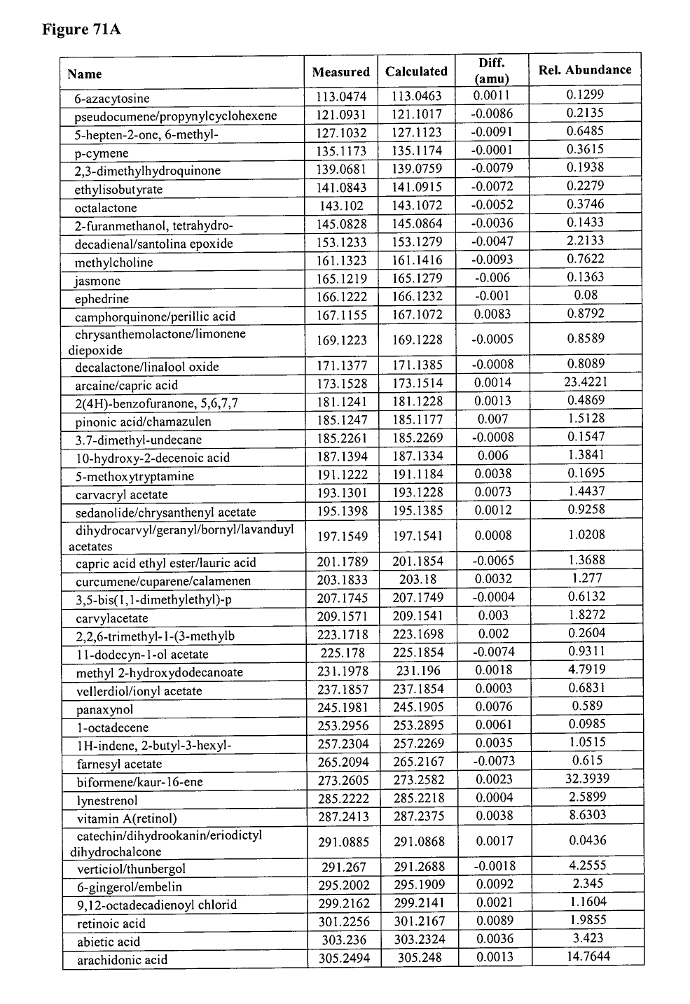

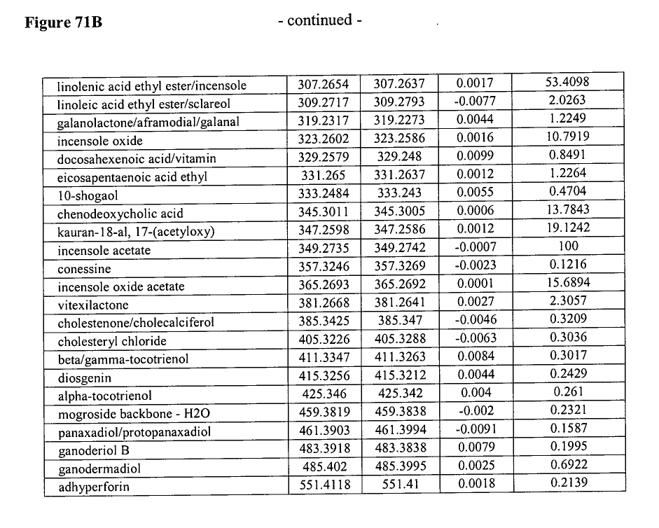

SILICA GEL 645 AT T=40° C., STAGE 1, P=100 BAR. The stage 1 extract contains no boswellic acids or acetylated boswellic acid derivatives as determined by DART-MS. Fatty acids, quinines, lactones, phytosterols, vitamins, gingerols, ganoderol, phenols, flavonoids, alkaloids, and shogaols are present in the stage 1 extract. See FIGS. 70 and 71.

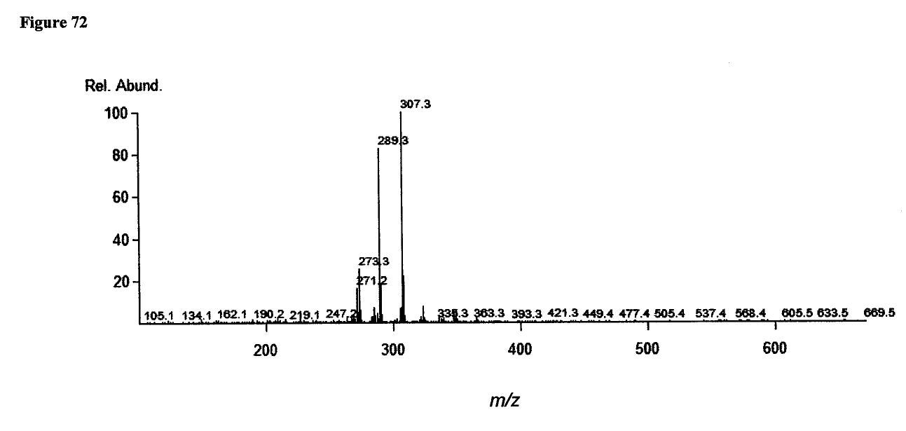

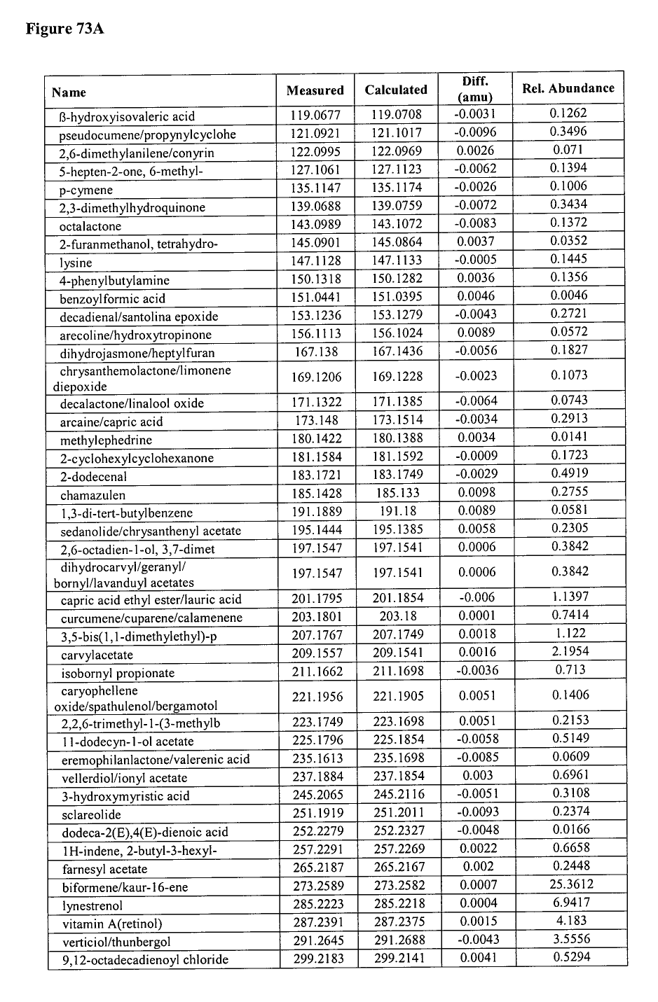

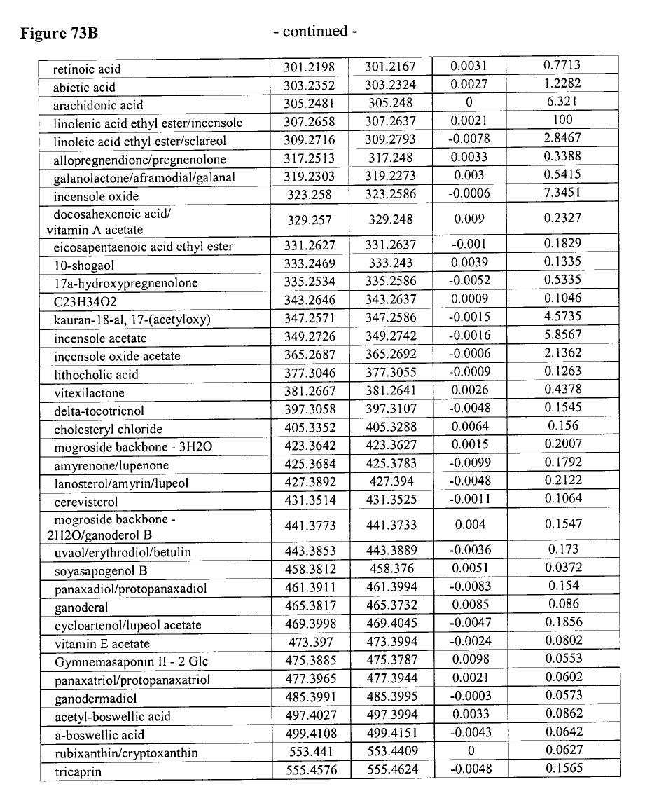

SILICA GEL 645 AT T=40° C., STAGE 2, P=150 BAR. BA and ABA are present in the stage 2 extract both at 0.1% relative abundance by DART-MS. Fatty acids, amino acids, quinones, lactones, alkaloids, terpenoids, phenols, shogaols and phytosterols are identified in the stage 2 extract. See FIGS. 72 and 73.

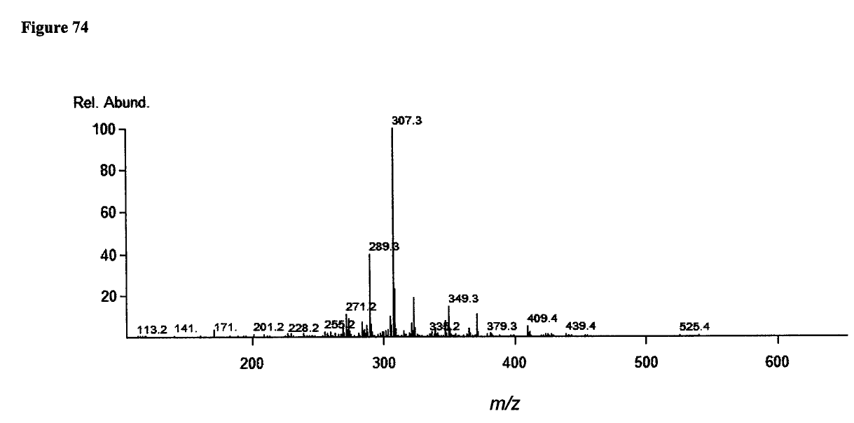

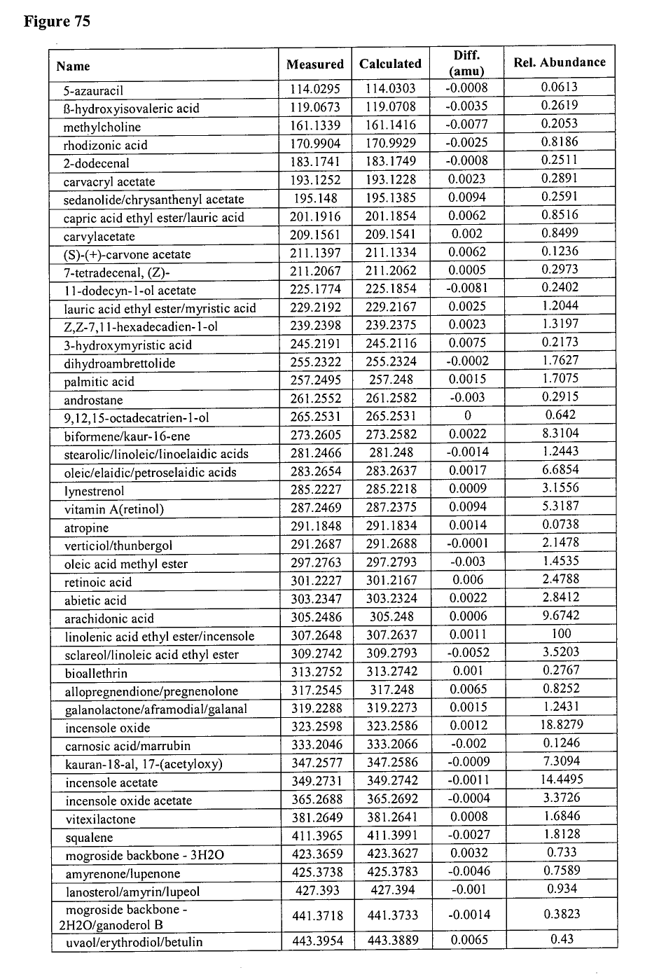

SILICA GEL 645 AT T=40° C., STAGE 3, P=200 BAR. No boswellic acids or acetylated boswellic acid derivatives are identified by DART-MS in the extract. Fatty acids, lactones, terpenoids, phenols, ganoderols, shogaols, phytosterols, vitamins, gingerols, alkaloids, and quinines are identified in the extract. See FIGS. 74 and 75.

### Example 4

EXAMPLE OF STEP 2 EXTRACTION AND FRACTIONATION OF THE LIPID SOLUBLE BOSWELLIA SPECIES CHEMICAL CONSTITUENTS USING SCCEP WITH SURFACE MODIFIED MESOPOROUS SILICA GEL AFFINITY ADSORBENTS. In typical experimental examples, 20 mL of Boswellia concentrated ethanol leaching extract (solute concentration about 600 mg/mL) were mixed with 30 gm silica gel (either TKR-methacrylate, TKR-STYR-ABS, or TKR-C18-15-100A) and then loaded into a SFE250 100 mL extraction vessel with both ends packed with glass wool. The extraction system was set at 40° C. The system was held in equilibrium at this temperature for 15 minutes. The CO₂ flow was instituted at a flow rate of 10 gm/min. The SCCEP pressure was sequentially changed from 90 bar to 200 bar to 200 bar with 5% ethyl acetate as a co-solvent. Each of the three stages was considered completed when no further extraction was obtained at the extraction sampling port. The samples at the completion of each processing stage were collected and weighed for yield computation. The residue obtained from the surface modified silica gel extractions were dissolved in ethyl acetate and then dried in a vacuum oven at 50° C. for 12 hours. The final residue products were white flakes that were analyzed by both GC-MS and HPLC. Typical results of the analyses are shown in FIG. 16.

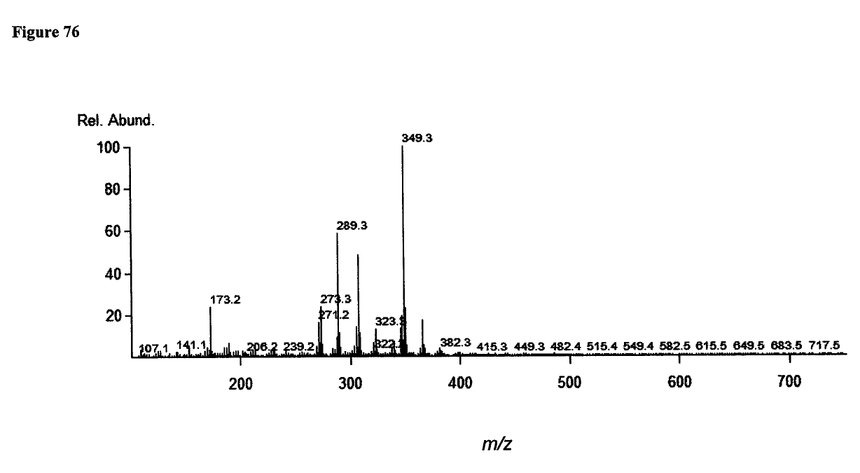

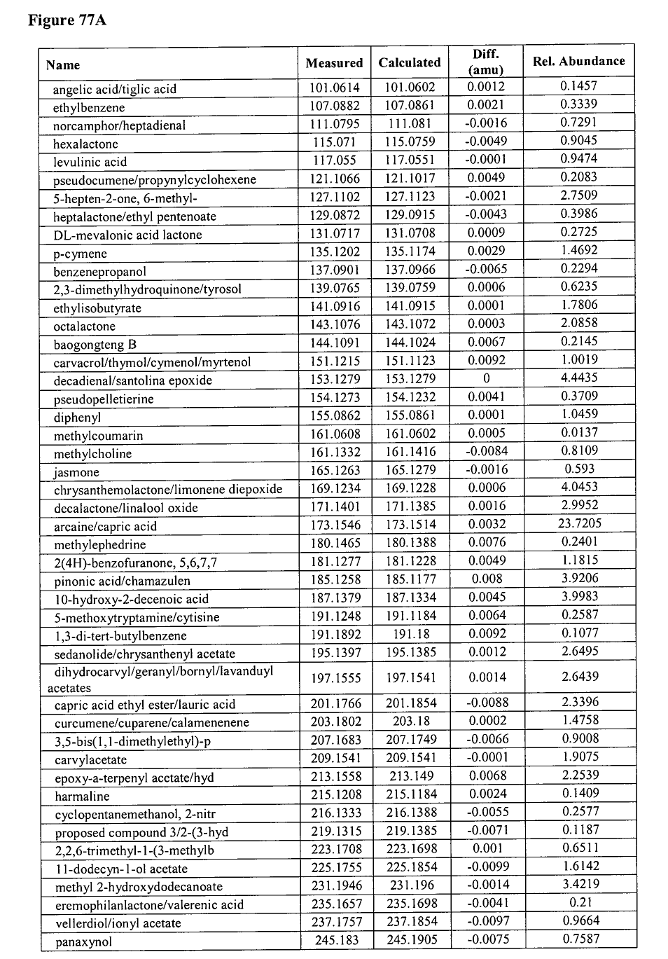

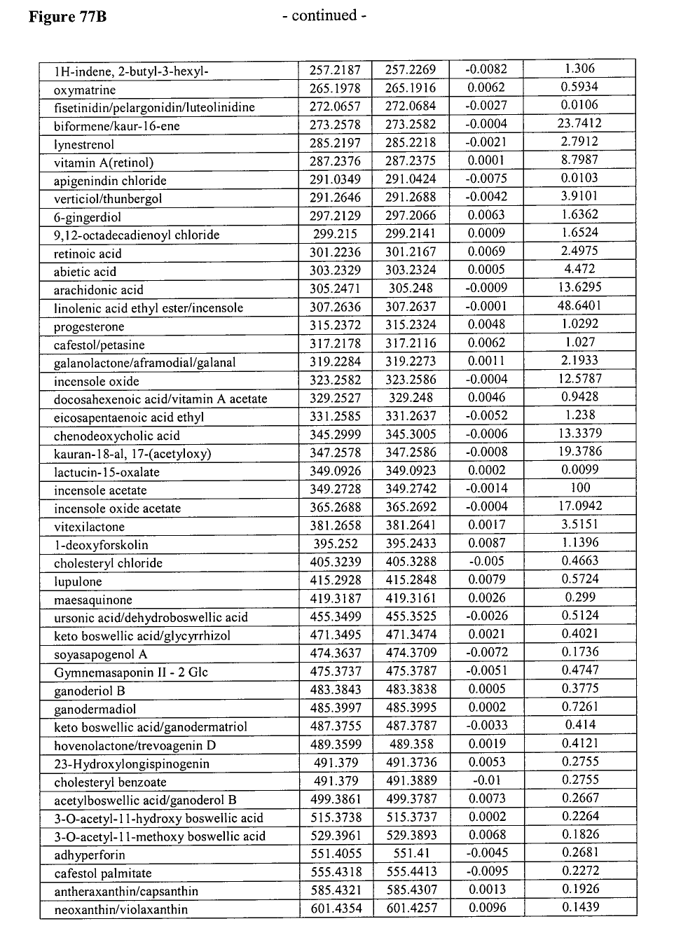

TKR-METHACRYLATE; T=40° C., STAGE 1, P=90 BAR. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 76 and 77.

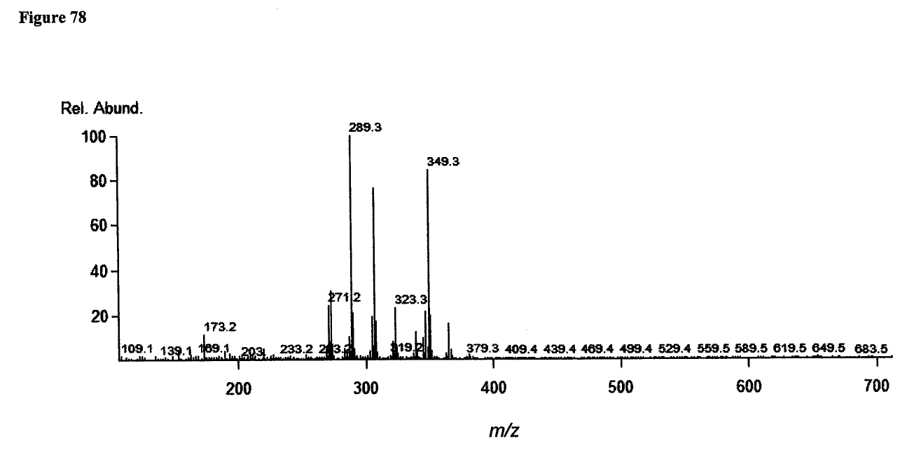

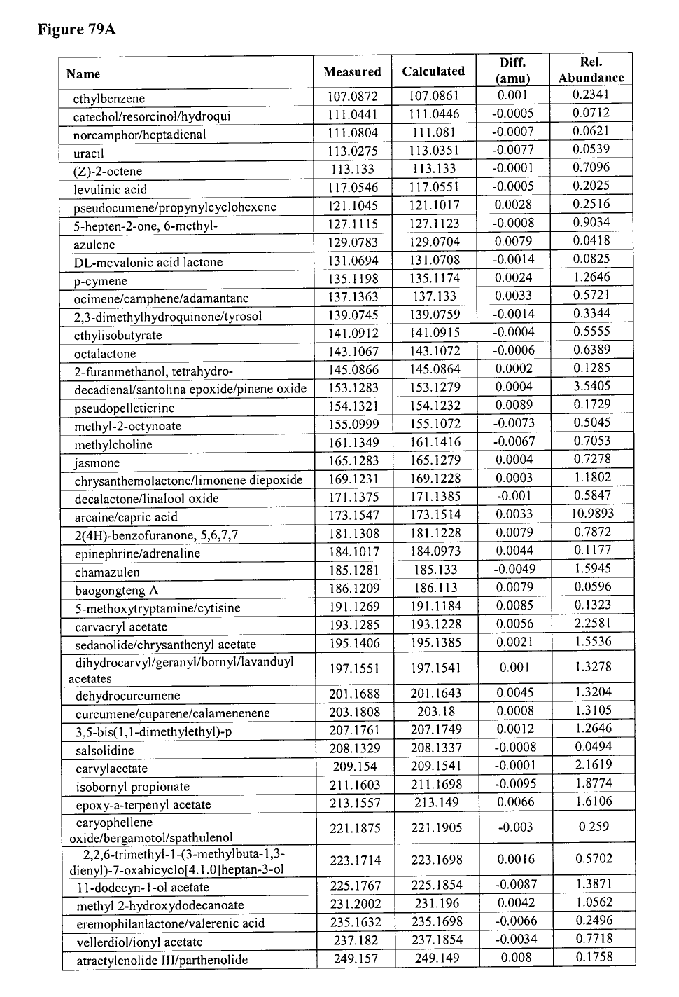

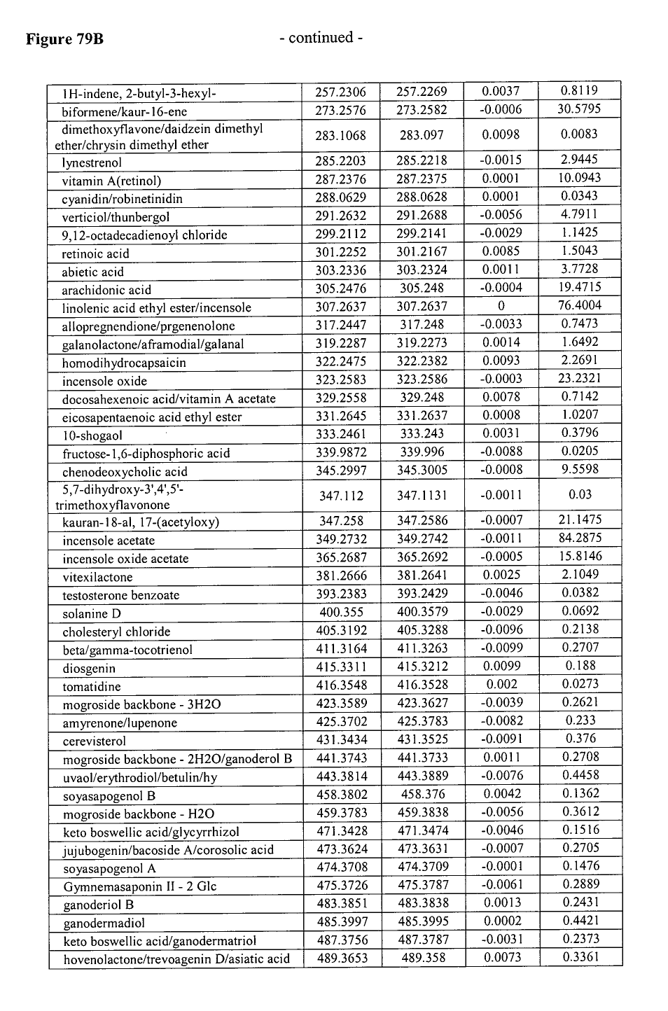

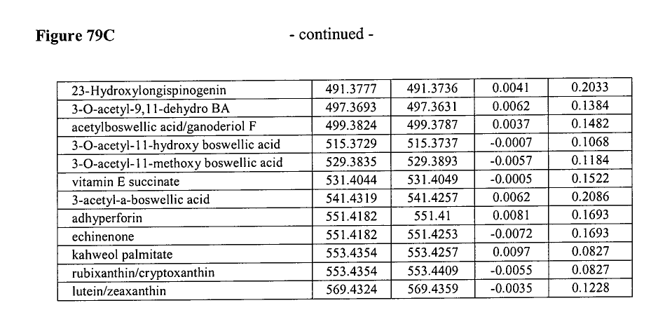

TKR-METHACRYLATE T=40° C., STAGE 2, P=200 BAR. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 78 and 79.

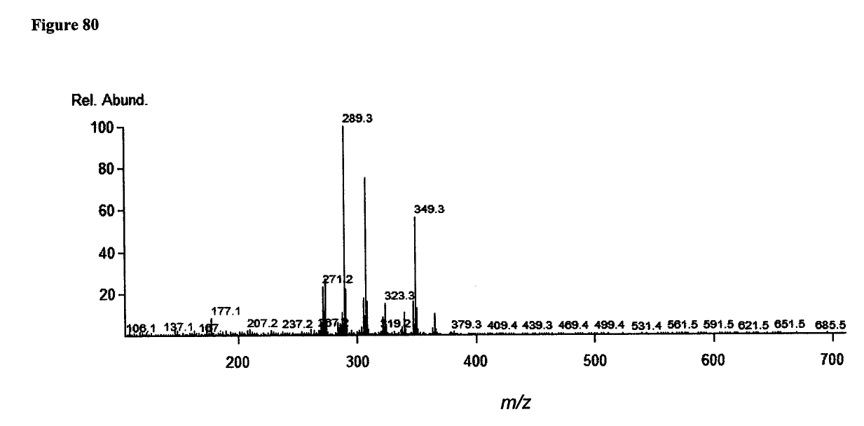

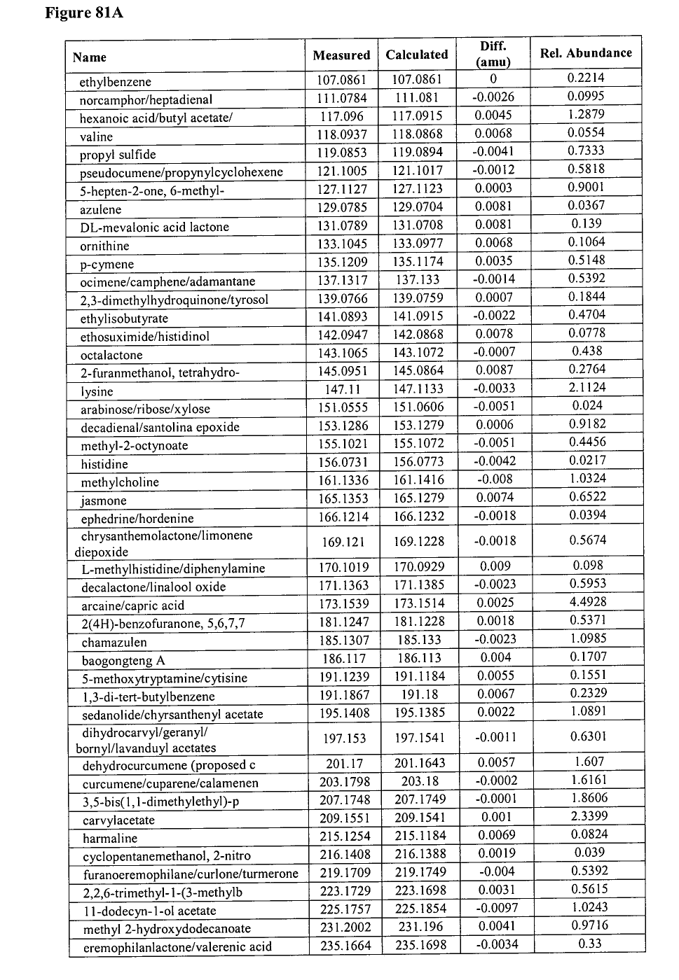

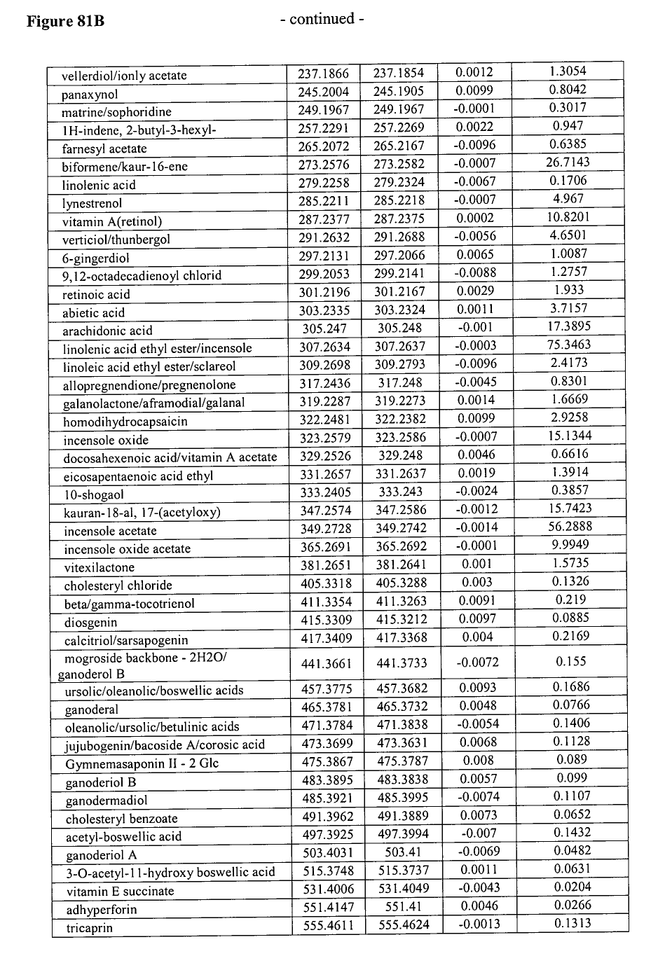

TKR-METHACRYLATE T=40° C., STAGE 3, P=200 BAR WITH 5% ETHYL ACETATE. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 80 and 81.

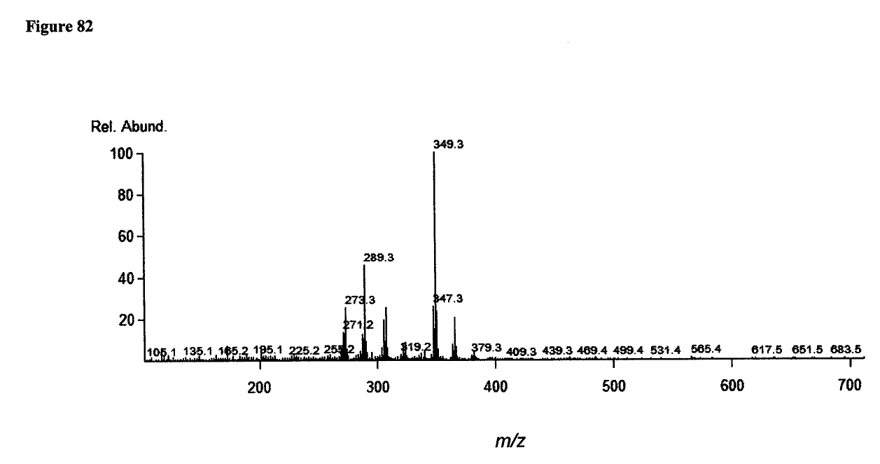

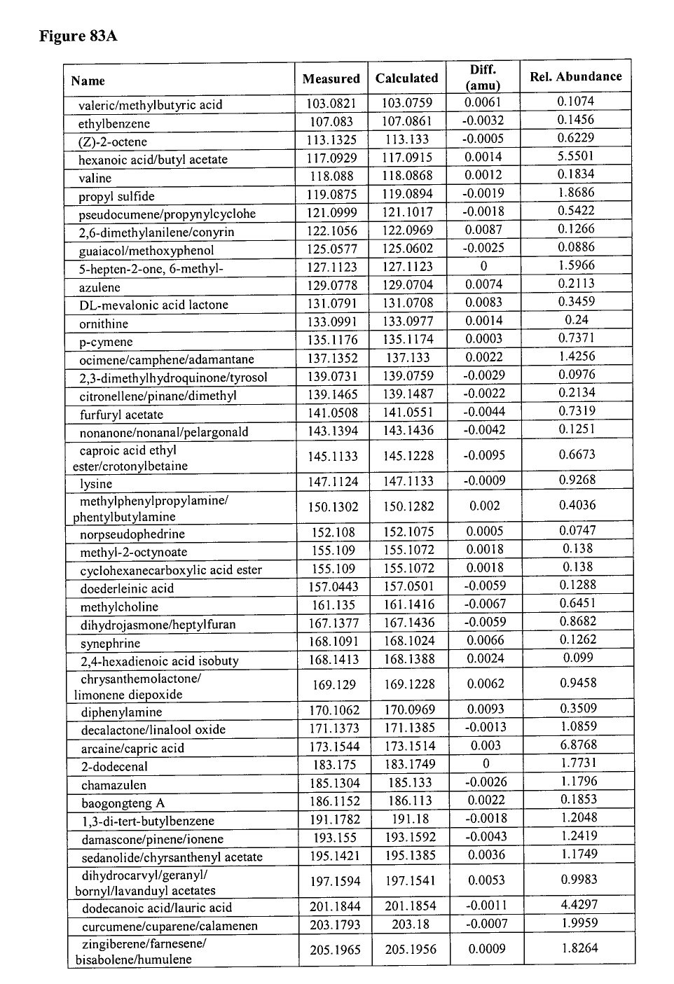

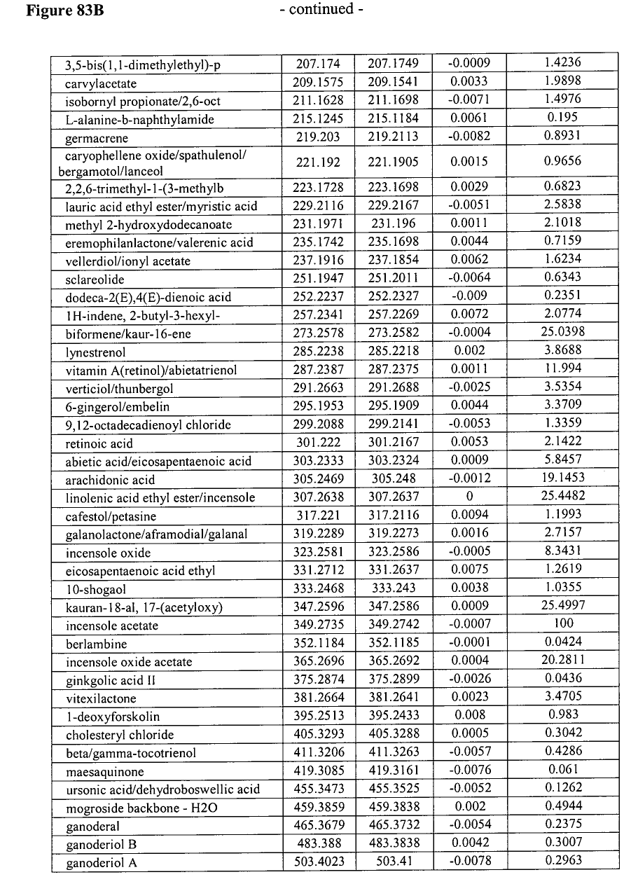

TKR-STYR-ABS T=40° C., STAGE 1, P=90 BAR. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 82 and 83.

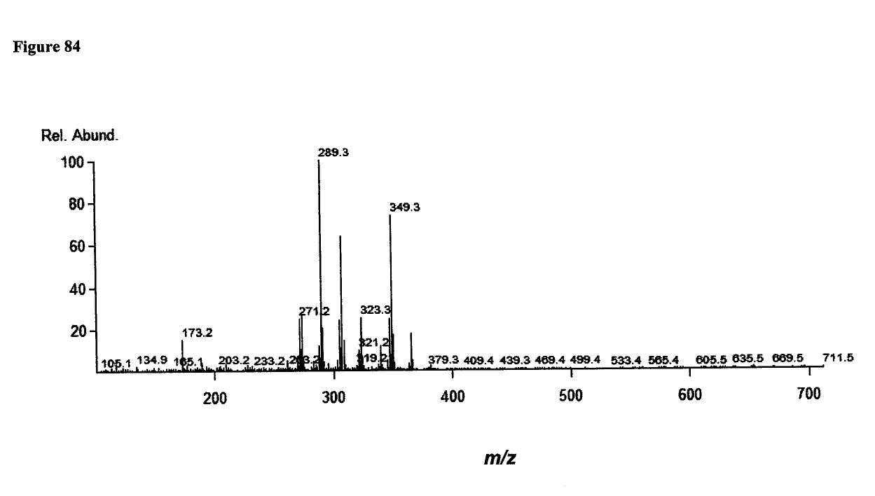

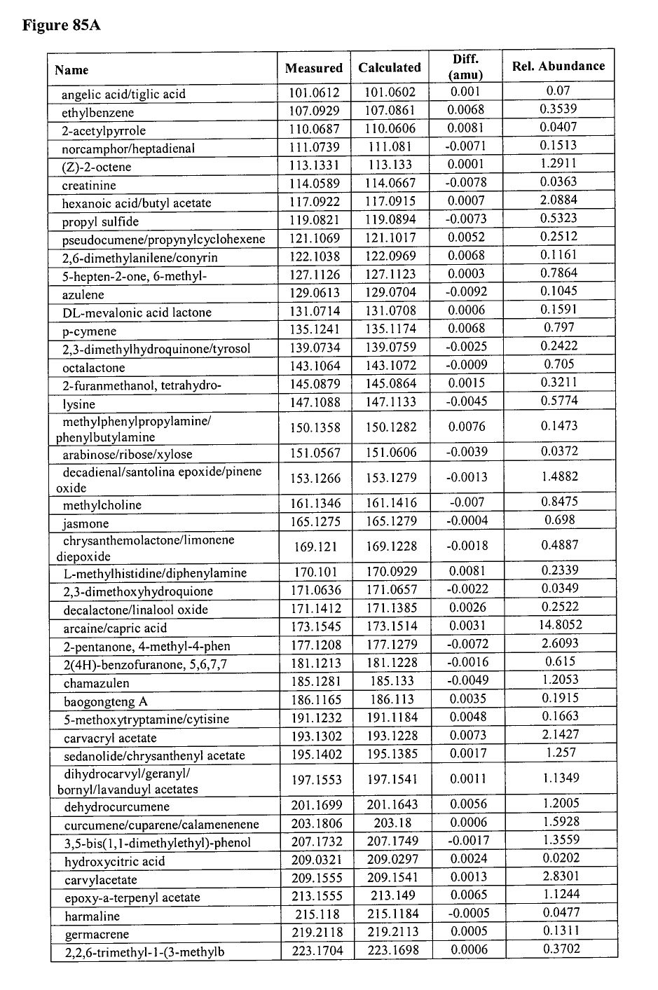

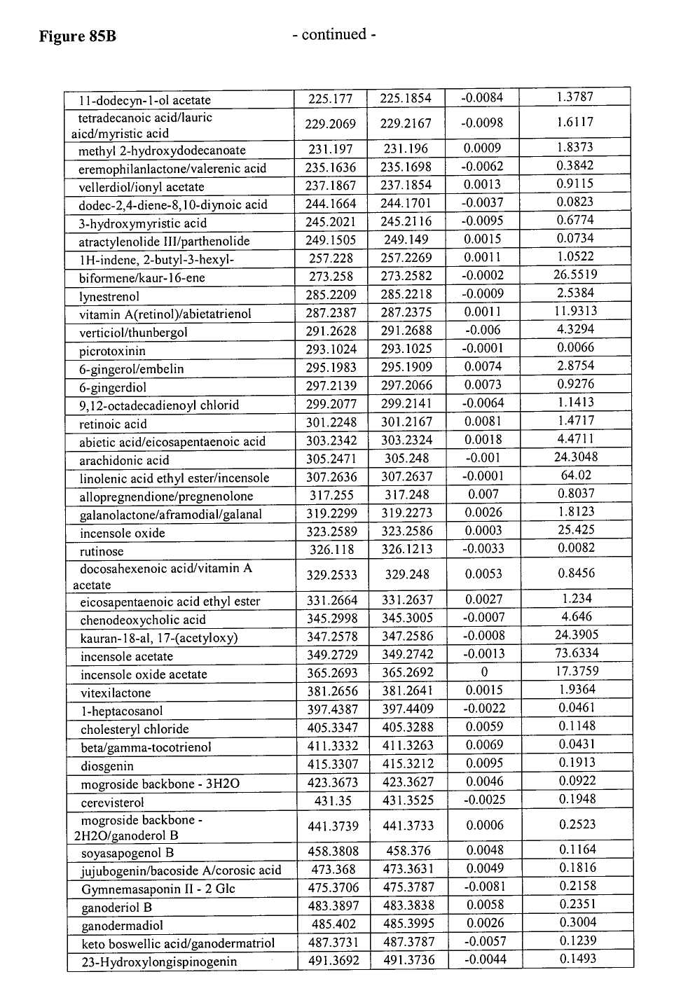

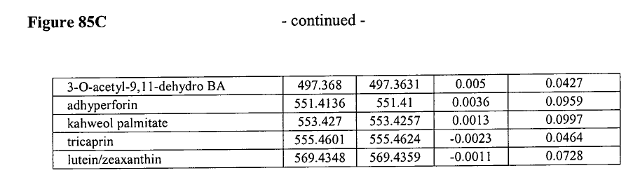

TKR-STYR-ABS T=40° C., STAGE 2, P=200 BAR. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 84 and 85.

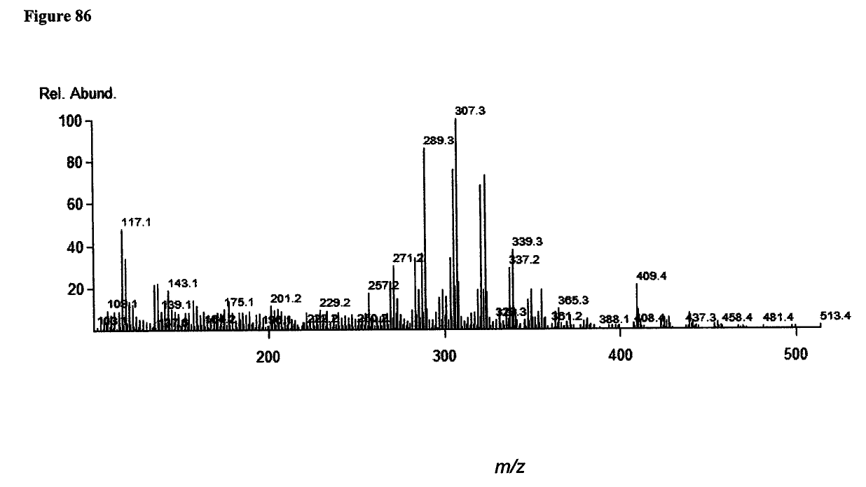

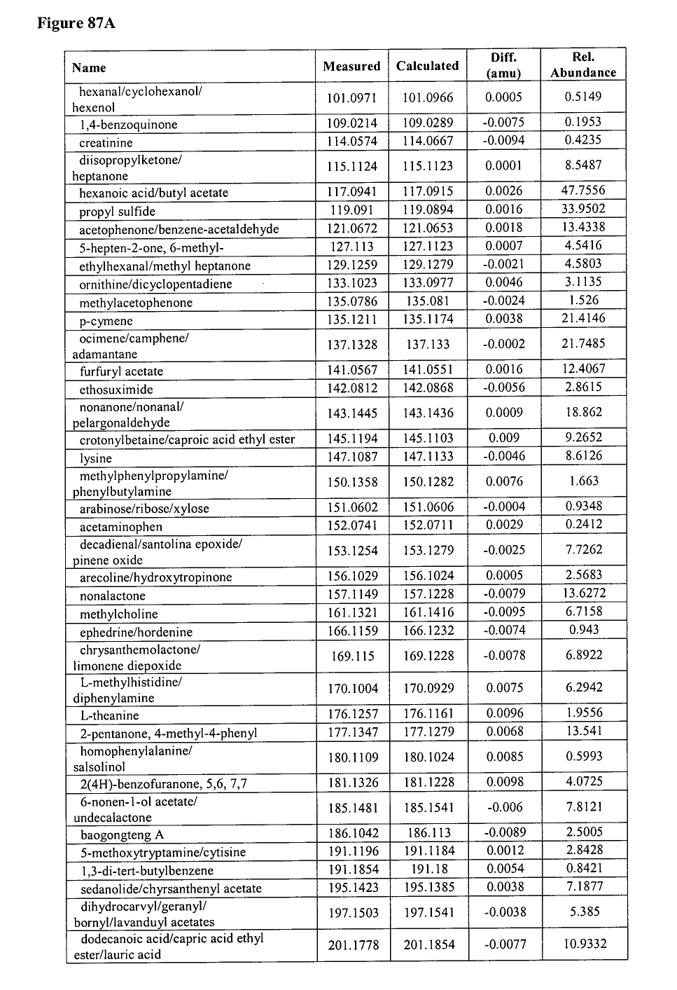

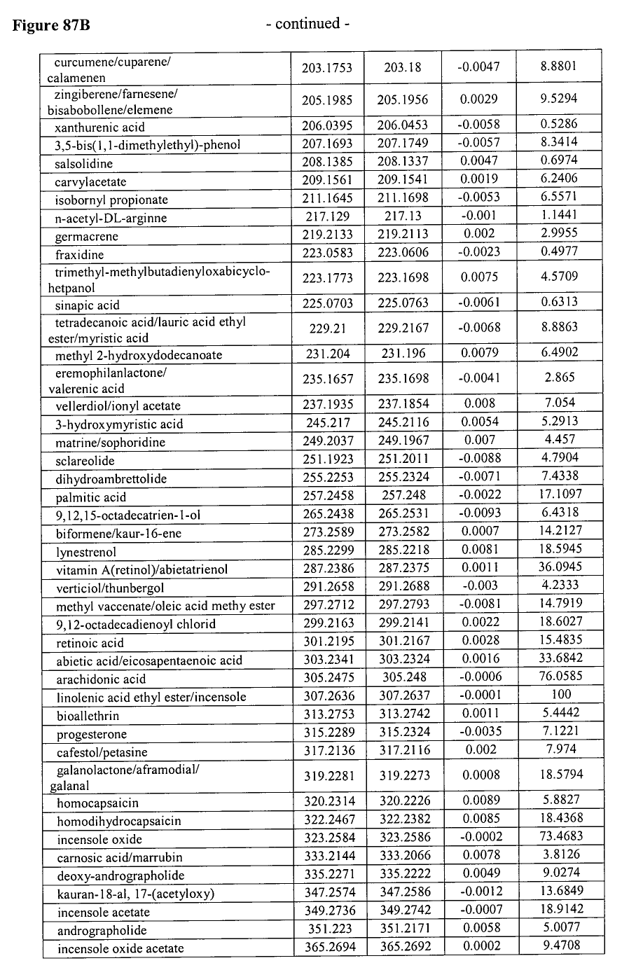

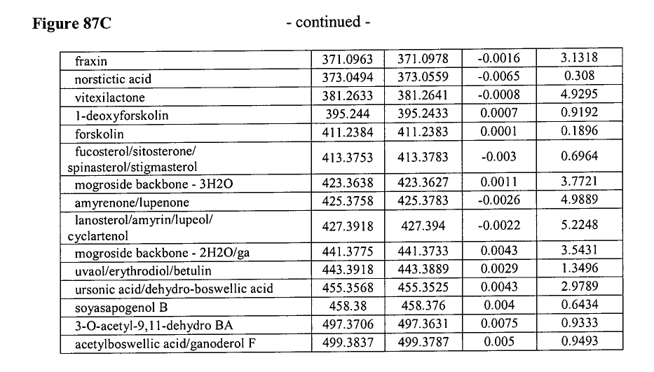

TKR-STYR-ABS T=40° C., STAGE 3, P=200 BAR WITH 5% ETHYL ACETATE. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 86 and 87.

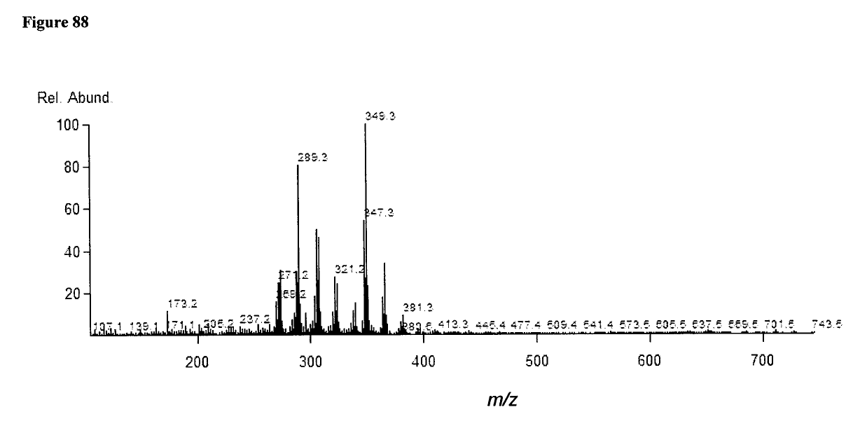

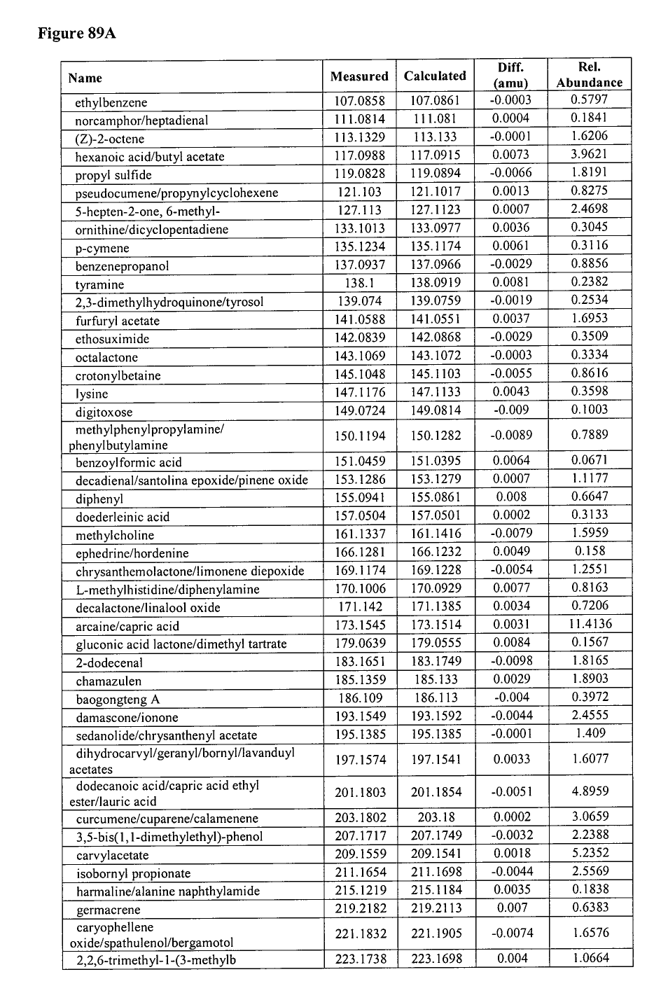

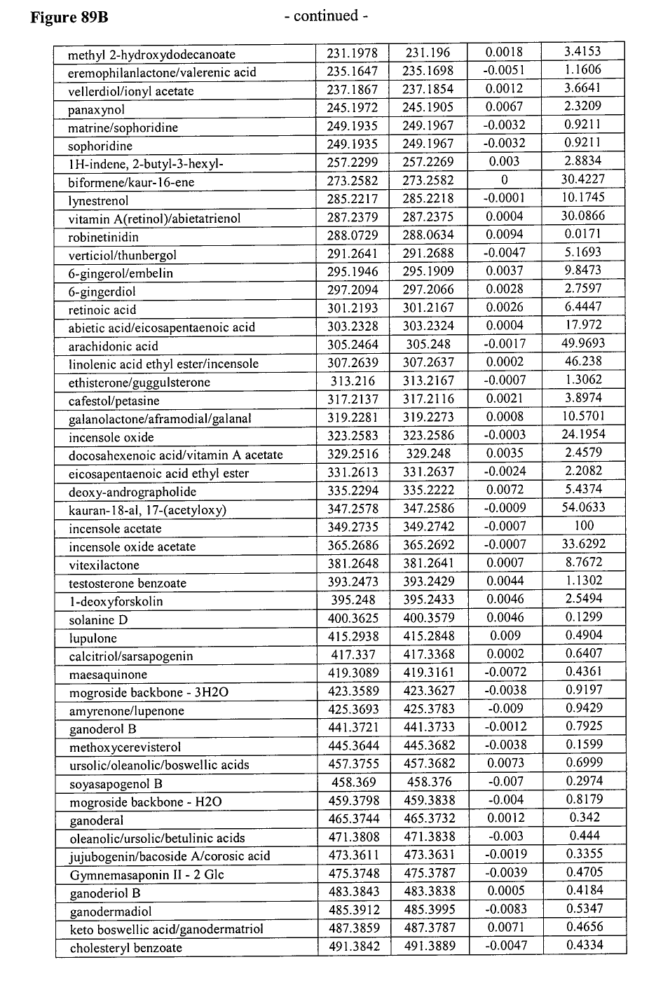

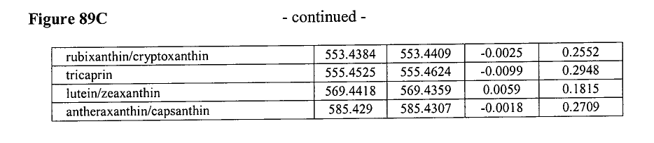

TKR-C18-15-100A T=40° C., STAGE 1, P=90 BAR. No boswellic acids or acetylated boswellic acid derivatives have been identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 88 and 89.

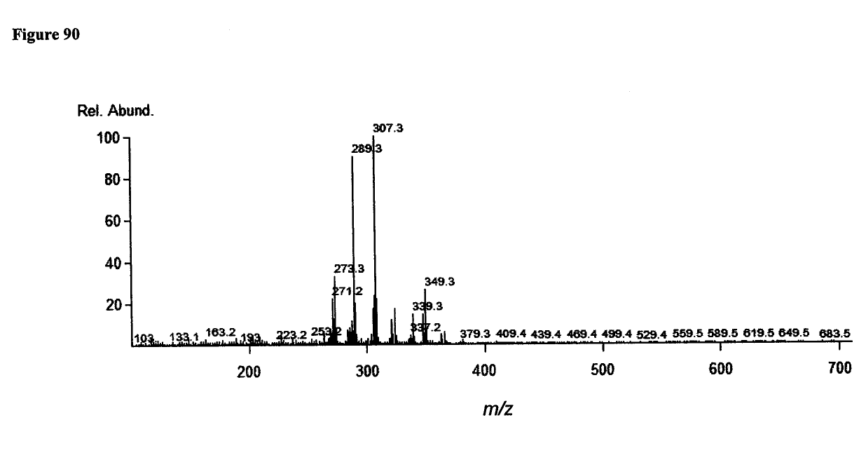

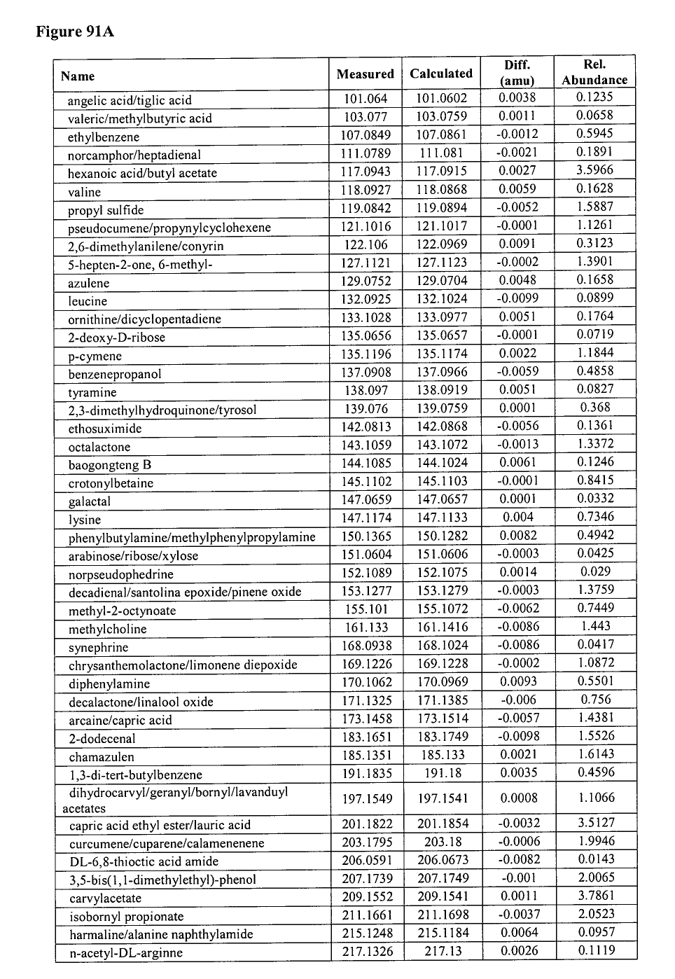

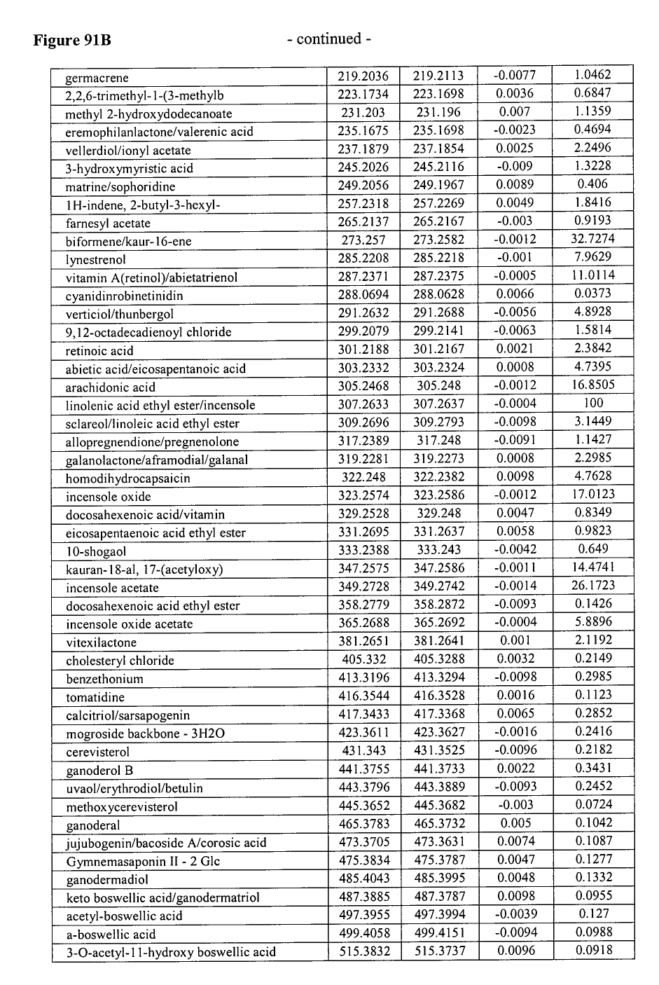

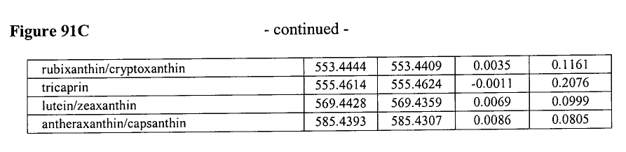

TKR-C18-15-100A T=40° C., STAGE 2, P=200 BAR. No boswellic acids or acetylated boswellic acid derivatives have been identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 90 and 91.

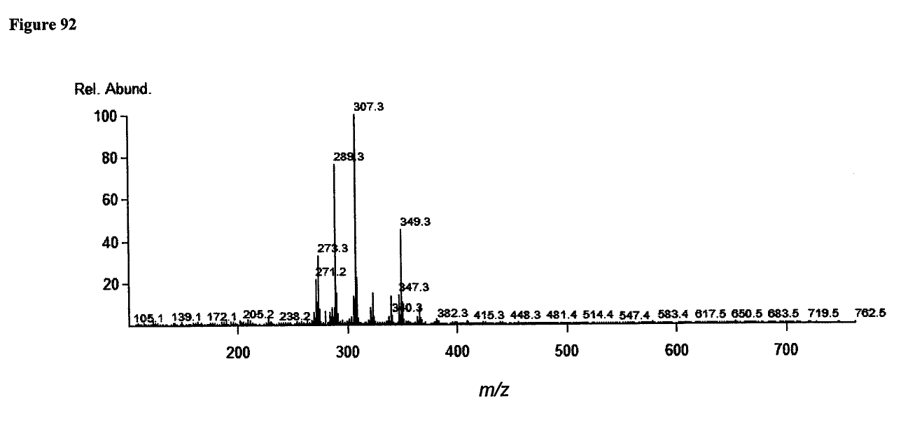

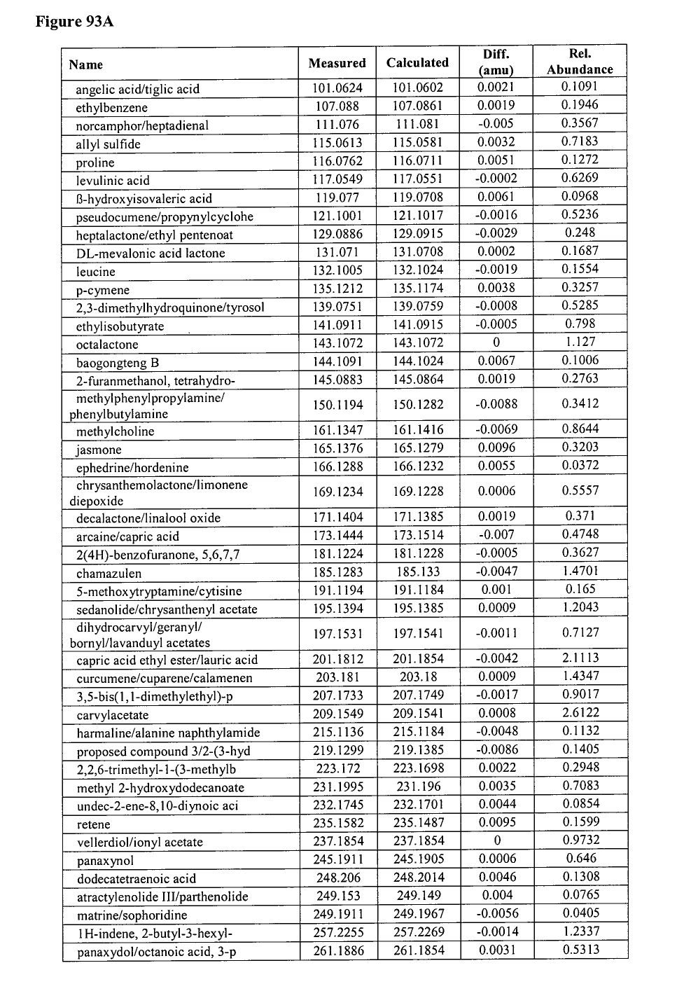

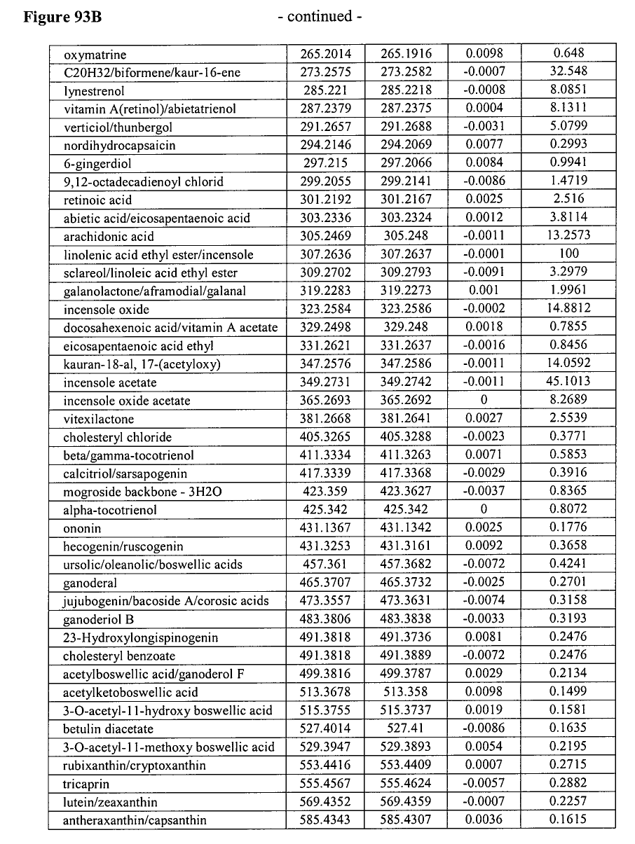

TKR-C18-15-100A T=40° C., STAGE 3, P=200 BAR WITH 5% ETHYL ACETATE. No boswellic acids or acetylated boswellic acid derivatives are identified by DART-MS in the S7 extract. Fatty acids, lactones, terpenoids, phenols, ganoderols, shogaols, phytosterols, vitamins, gingerols, alkaloids, and quinines are identified in the S7 extract. See FIGS. 92 and 93.

### Example 5

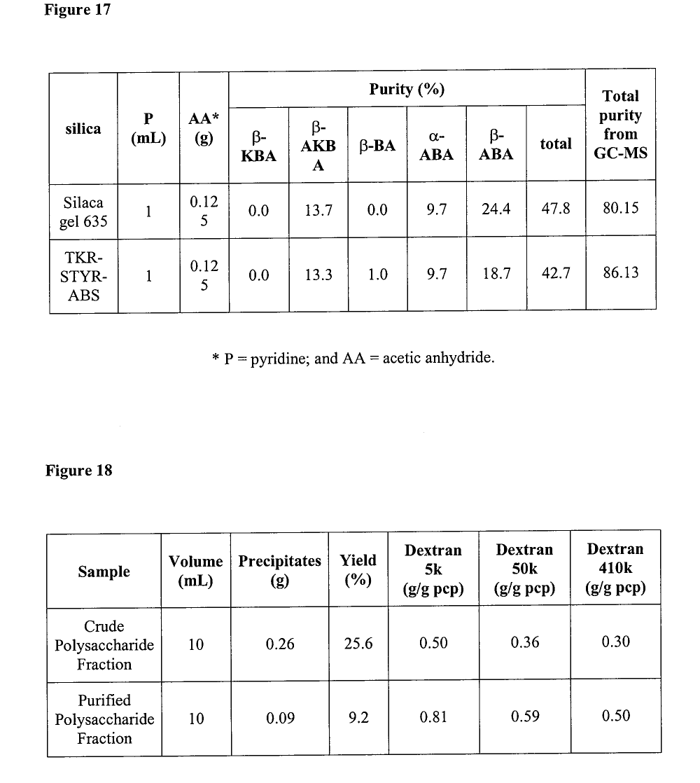

EXAMPLE OF STEP 3 ACETYLATION OF PURIFIED BOSWELLIC ACID FRACTION. In a typical example of the acetylation of a purified boswellic acid fraction, 0.1 gm of purified boswellic acid fraction from Step 2 SCCEP processing was dissolved in 1 mL of pyridine solution. To this mixture, 0.125 gm of acetic anhydride was added. This mixture was stirred for 19 hours and then quenched with 10 gm of distilled water ice. The phases were separated and the aqueous phase was extracted with diethyl ether three times. The combined diethyl ether extractions were combined and dried in a vacuum oven. The dried product was the acetylated purified boswellic acid fraction. Typical results are shown in FIG. 17.

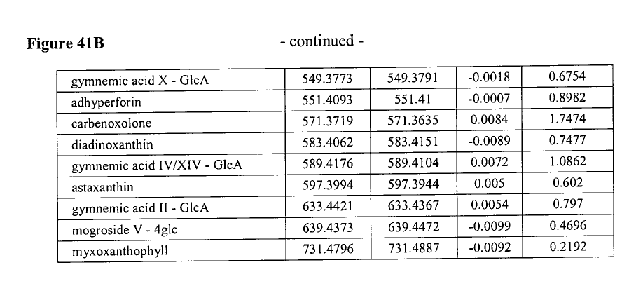

ACETYLATION OF BOSWELLIA CARTERI EXTRACT HS700. The reaction solvent DMAP is the most abundant peak in HS710, most likely due to insufficient drying of the reaction products. Many of the previously discussed compounds are present in HS708 including biformene, vitamin A, arachidonic acid, incensole (and incensole derivatives), acetyloxykauranal, norhydrocapsaicin, gingerols, ganoderols, ganoderic acids, shogaols, and gymnemic acids. Some xanthin derived compounds are present between m/z [M+H]+569.4 and 585.4. The boswellic acids identified in HS708 include DHBA, BA, KBA, ADHBA, ABA, AKBA, and AHBA. See FIGS. 40 and 41.

### Example 6

EXAMPLE OF STEP 5 POLYSACCHARIDE FRACTION EXTRACTION. A typical experimental example of solvent extraction and precipitation of the water soluble, ethanol insoluble purified polysaccharide fraction chemical constituents of Boswellia species is as follows: 100 gm of the solid residue from the ethanol leaching extraction (Step 1) was extracted using 1000 mL of distilled water for two hours at 70° C. Absolute ethanol was added to 10 mL of the extraction solution to make a final solution concentration of 80% ethanol. This solution was centrifuged at 2,000 rpm for 10 minutes and decanted. The “crude” polysaccharide precipitate was collected and dried in a vacuum oven at 50° C. for 12 hours. The supernatant was discarded. The dried crude polysaccharide fraction was dissolved in 10 mL of distilled water. An aliquot of 40 mL of absolute ethanol was added to make an 80% ethanol solution. This solution was then centrifuged at 2,000 rpm and decanted. The purified polysaccharide fraction was collected and vacuum oven dried at 50° C. for 12 hours. The dried polysaccharide fractions were weighed, yield calculated, and dissolved in water for analysis of polysaccharide purity with the colormetric method using dextran as reference standards. Moreover, AccuTOF-DART mass spectrometry was used to further profile the molecular weights of the compounds comprising the purified polysaccharide fraction. The results are shown in FIG. 18.

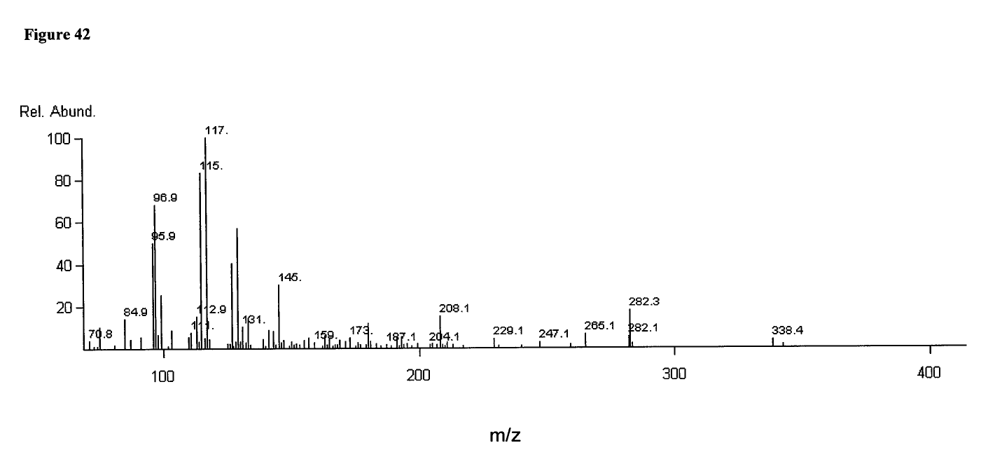

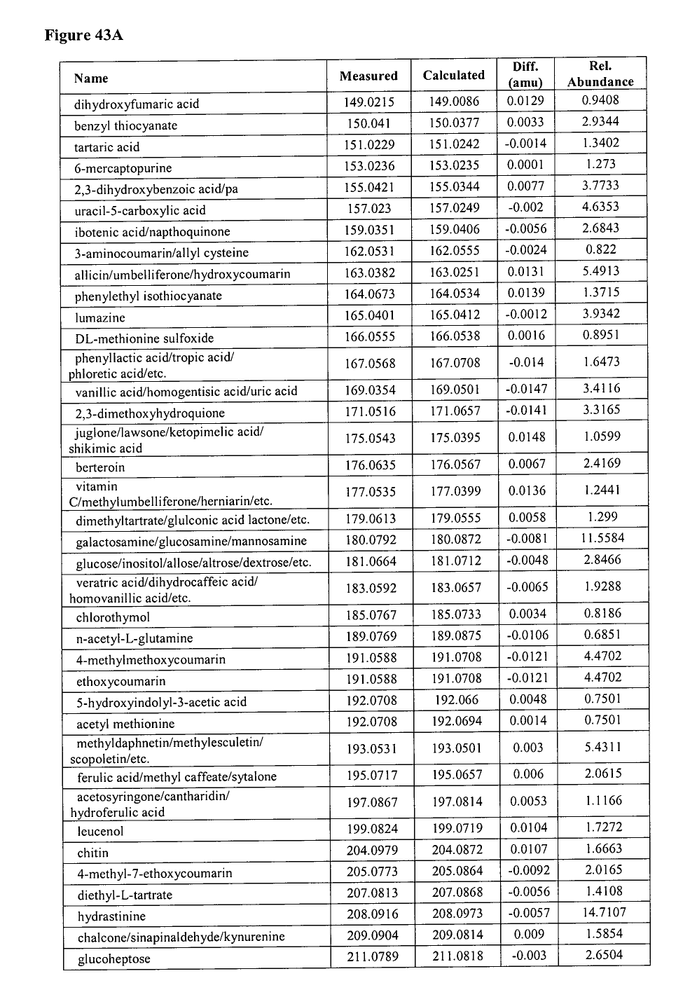

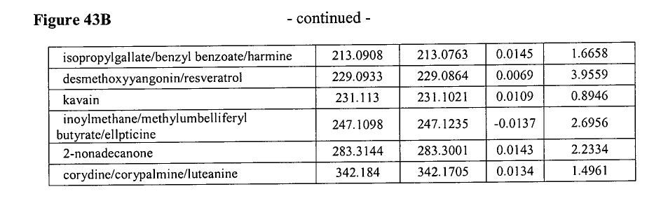

For a Boswellia carteri polysaccharide DART analysis see FIGS. 42 and 43.

### Example 7

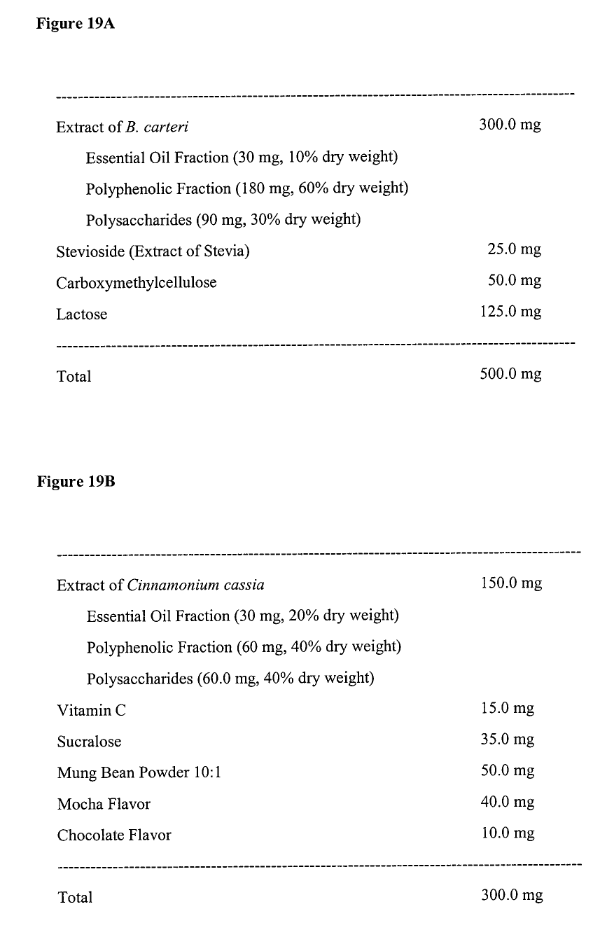

The ingredients in FIG. 19A are mixed to form a formulation. The novel extract of Boswellia species comprises an essential oil fraction, phenolic acid-essential oil fraction, and polysaccharide fraction by % mass weight greater than that found in the natural rhizome material or convention extraction products. The formulations can be made into any oral dosage form and administered daily or to 15 times per day as needed for the physiological and psychological effects desired (enhanced brain function and analgesia) and medical effects (non-insulin dependent diabetes mellitus, anti-platelet aggregation and anti-thrombosis, cardiovascular and cerebrovascular disease prevention and treatment, anti-atherosclerosis, anti-hypercholesterolemia, cardiac protection, nervous system protection, anti-inflammatory, anti-allergic, anti-arthritis, anti-rheumatic, anti-gout, gastro-intestinal disorders, cough, common cold, fever, lipolytic, improved wound healing, anti-bacterial, anti-fungal, and anti-cancer).

### Example 8

The ingredients in FIG. 19B were mixed to form a formulation. The novel extract composition of Boswellia chuangxiong comprises an essential oil, phenolic acid-essential oil, and polysaccharide chemical constituent fractions by % mass weight greater than that found in the natural plant material or conventional extraction products. The formulation can be made into any oral dosage form such as, but not limited to, an oral rapid dissolve tablet for oral absorption and administered safely up to 15 times per day as needed for the physiological, psychological and medical effects desired (see Example 1, above).

### REFERENCES

For reference in the text, when number appears in parentheses (#), the numbers refers to the following references:
1. Culioli G et al. Phytochemistry 62:537, 2003.
2. Ammon H P. Wien Med Wochenschr 152(15-16):373, 2002.
3. Shao Y et al. Planta Med 64:328, 1998.
4. Hamm S et al. J Chromatography, A 1018(1):73, 2003.
5. Buchelle B et al. J Chromatography B 791:21, 2003.
6. Winterstein A et al. Z Physiol Chem 208:9, 1932.
7. Pardy R S & Bhattacharyya S C. Indian J Chem 16B: 176, 1978.
8. Fattorusso E et al. Phytochem 22:2868, 1983.
9. Koch A & Hahn-Deinstrop E. Bioforum 6:352, 1998.
10. Krohn K et al. Phytochem Anal 12:374, 2001.
11. Han B H et al. Arch Pharm Res 8:213, 1985.
11a. Xaasan C F et al. Rend Acad Sci Fis Mat 51:93, 1984.
11b. Zhou J Y et al. Chinese Chem Lett 13:65, 2002.
12. Etzel R. Phytomed 3(1):91, 1996.
13. Safayhi H et al. Planta Med 66:110, 2000.
14. Schweitzer S et al. J Nat Prod 63:1058, 2000.
15. Badria F A et al. Z Naturforsch [C] 58(7-8), 2003.
16. Sharma S et al. Phytomed 11(2-3):255, 2004.
17. Singh G B et al. Phytomed 3(1):87, 1996
18. Singh G B et al. Phytomed 3(1):81, 1996.
19. Tripathi Y B et al. Inflammopharmacology 12(2):131, 2004.
20. Darshan S & Doreswamy R. Phytother Res 18(5):343, 2004.
21. Kimmatkar N et al. Phytomed 10(1), 3, 2003.
22. Kulkarni R R et al. J Ethnopharmacol 33(1-2), 1991.
23. Reichling J et al. Schweiz Arch Tierheilkd 146(2):71, 2004.
24. Gupta I et al. Eur J Med Res 3(11):511, 1997.
25. Jaber R. Prim Care 29(2):231, 2002.
26. Gupta I et al. Eur J Med Res 2(1):37, 2002.
27. Gupta I et al. Planta Med 67(5):391, 2001.
28. Gerhardt H et al. Zeitshrift fur Gastroenterologie 39(1):11, 2001.
29. Krieglstein C F et al. Int J Colorectal Dis 16(2):88, 2001.
30. Parks Y S et al. Adv Exp Med Biol 507:387, 2002.
31. Zhao W et al. Cancer Detect Prev 27(1):67, 2003.
32. Hostanska K et al. Anticancer Res 22(5):2853, 2002.
33. Wallace J M. Integr Cancer Ther 1(1):7, 2002.
34. Liu J J et al. Carcinogenesis 23(12):2087, 2002.
35. Mikhaeil B R et al. Z Naturforsch [C] 58(3-4):230, 2003.
36. Pungle P et al. Indian J Exp Biol 41(12):1460, 2003.
37. Badria F A et al. Biosciences, Biotech Res Asia 1(1):1, 2003.
38. Safayhi H et al. J Pharmacol Exp Therapeutics 281(1):460, 1997.
39. Park Y S et al. Planta Med 68(5):397, 2002.
40. Park Y S et al. Adv Exp Med Biol 507(5):387, 2002.
41. Syrovets T et al. Molecular Pharmacol 58(1):71, 2000.
42. Bowellia (Boswellia serrata). Harvard Medical School. http://www.intelihealth.com.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A composition comprising α- and/or β-boswellic acid and/or their C-acetates in an amount greater than 65% by weight.

2. The composition of claim 1, wherein the α- and β-boswellic acids and/or their C-acetates comprise alpha-boswellic acid (α-BA), acetyl-alpha-boswellic acid (α-ABA), beta-boswellic acid (β-BA), acetyl-beta-boswellic acid (β-ABA), 9.11-dehydro-alpha-boswellic acid (D-β-BA), acetyl-9.11-dehydro-alpha-boswellic acid (D-α-ABA), 9.11-dehydro-beta-boswellic acid (D-β-BA), acetyl-9.11-dehydro-beta-boswellic acid (D-β-ABA), lupeolic acid (LA), acetyl-lupeolic acid (ALA), 11-keto-beta-boswellic acid (β-KBA), or acetyl-11-keto-beta-boswellic acid (β-AKBA).

3. The composition of claim 1, wherein the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

4. The composition of claim 1, wherein the amount of α- and/or β-boswellic acid and/or their C-acetates is greater than 70, 75, 80, 85, 90, or 95% by weight.

5. The composition of claim 1, further comprising n-octyl acetate, incensole, incensole acetate, linalol, borneol, camphene, elemene, caryophyllene, incensole oxide, incensole oxide acetate, or combinations thereof.

6. The composition of claim 5, wherein the α- and β-boswellic acids and/or their C-acetates comprise α-BA, α-ABA, β-BA, β-ABA, D-α-BA, D-α-ABA, D-β-BA, D-β-ABA, LA, ALA, β-KBA, or β-AKBA.

7. The composition of claim 5, wherein the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

8. The composition of claim 5, wherein the amount of n-octyl acetate, incensole, incensole acetate, linalol, borneol, camphene, elemene, caryophyllene, incensole oxide, incensole oxide acetate, or combinations thereof is 5, 10, 15, 20, 25, or 30 by weight.

9. The composition of claim 5, further comprising 1,3-di-t-butylbenzene, 1-undecanol, dodecanoic acid, 4-tetradecanol, viridiflorol, nerodidol isobutyrate, octadecanoic acid, butyl acetate, dimer of alpha-phellandrene, alpha-amyrenone, beta-amyrin, 3-epi-alpha-amyrin, 3-epi-beta-amyrin, or lupeenone.

10. The composition of claim 5, wherein the amount of α- and/or β-boswellic acid and/or their C-acetates is greater than 70, 75, 80, 85, 90, or 95% by weight.

11. The composition of claim 1, further comprising a polysaccharide.

12. The composition of claim 11, wherein the polysaccharide comprises dextran, glucose, arabinose, galactose, rhamnose, xylose or uronic acid.

13. The composition of claim 11, wherein the polysaccharide comprises dextran, glucose, arabinose, galactose, rhamnose, xylose and uronic acid.

14. The composition of claim 11, wherein the α- and β-boswellic acids and/or their C-acetates comprise α-BA, α-ABA, β-BA, β-ABA, D-α-BA, D-α-ABA, D-β-BA, D-β-ABA, LA, ALA, β-KBA, or β-AKBA.

15. The composition of claim 11, wherein the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

16. The composition of claim 11, wherein the amount of polysaccharide is 5, 10, 15, 20, 25, 30, or 35% by weight.

17. The composition of claim 11, further comprising 1,3-di-t-butylbenzene, 1-undecanol, dodecanoic acid, 4-tetradecanol, viridiflorol, nerodidol isobutyrate, octadecanoic acid, butyl acetate, dimer of alpha-phellandrene, alpha-amyrenone, beta-amyrin, 3-epi-alpha-amyrin, 3-epi-beta-amyrin, or lupeenone.

18. The composition of claim 11, wherein the amount of α- and/or β-boswellic acid and/or their C-acetates is greater than 70, 75, 80, 85, 90, or 95% by weight.

19. The composition of claim 1, further comprising n-octyl acetate, incensole, incensole acetate, linalol, borneol, camphene, elemene, caryophyllene, incensole oxide, incensole oxide acetate, or combinations thereof, and a polysaccharide.

20. The composition of claim 19, wherein the α- and β-boswellic acids and/or their C-acetates comprise α-BA, α-ABA, β-BA, β-ABA, D-α-BA, D-α-ABA, D-β-BA, D-β-ABA, LA, ALA, β-KBA, or β-AKBA.

21. The composition of claim 19, wherein the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

22. The composition of claim 19, wherein the amount of n-octyl acetate, incensole, incensole acetate, linalol, borneol, camphene, elemene, caryophyllene, incensole oxide, incensole oxide acetate, or combinations thereof is 5, 10, 15, 20, or 25 by weight.

23. The composition of claim 19, wherein the polysaccharide comprises dextran, glucose, arabinose, galactose, rhamnose, xylose or uronic acid.

24. The composition of claim 19, wherein the polysaccharide comprises dextran, glucose, arabinose, galactose, rhamnose, xylose and uronic acid.

25. The composition of claim 19, wherein the amount of polysaccharide is 5, 10, 15, 20, or 25% by weight.

26. The composition of claim 19, further comprising 1,3-di-t-butylbenzene, 1-undecanol, dodecanoic acid, 4-tetradecanol, viridiflorol, nerodidol isobutyrate, octadecanoic acid, butyl acetate, dimer of alpha-phellandrene, alpha-amyrenone, beta-amyrin, 3-epi-alpha-amyrin, 3-epi-beta-amyrin, or lupeenone.

27. The composition of claim 19, wherein the amount of α- and/or β-boswellic acid and/or their C-acetates is greater than 70, 75, 80, 85, 90, or 95% by weight.

28. A composition comprising 30-70% by weight α- and/or β-boswellic acid and/or their C-acetates; 5-25% by weight n-octyl acetate, incensole, incensole acetate, linalol, borneol, camphene, elemene, caryophyllene, incensole oxide, incensole oxide acetate, or combinations thereof, and 5-50% by weight polysaccharide.

29. The composition of claim 28, wherein the amount of α- and/or β-boswellic acid and/or their C-acetates is 40-60% by weight; the amount of n-octyl acetate, incensole, incensole acetate, linalol, borneol, camphene, elemene, caryophyllene, incensole oxide, incensole oxide acetate, or combinations thereof is 10-20% by weight; and the amount of polysaccharide is 30-40% by weight.

30. The composition of claim 29, wherein the α- and β-boswellic acids and/or their C-acetates comprise α-BA, α-ABA, β-BA, β-ABA, D-α-BA, D-α-ABA, D-β-BA, D-β-ABA, LA, ALA, β-KBA, or β-AKBA.

31. The composition of claim 29, wherein the α- and β-boswellic acids and their C-acetates comprise α-BA, α-ABA, β-BA, β-ABA, D-α-BA, D-α-ABA, D-β-BA, D-β-ABA, LA, ALA, β-KBA, and β-AKBA.

32. The composition of claim 29, wherein the polysaccharide comprises dextran, glucose, arabinose, galactose, rhamnose, xylose or uronic acid.

33. The composition of claim 29, wherein the polysaccharide comprises dextran, glucose, arabinose, galactose, rhamnose, xylose and uronic acid.

34. A pharmaceutical composition comprising the composition of claim 1 and a pharmaceutical carrier.

35. A method for making a boswellia species extract having at least one predetermined characteristic comprising:
a. sequentially extracting a boswellia species plant material to yield an essential oil fraction, a boswellic acid or acetylated boswellic acid fraction, and a polysaccharide fraction, wherein
b. the lipid soluble chemical constituents are derived by extracting plant feedstock material by solvent extraction;
c. the essential oil fraction or sub-fractions is extracted from the lipid soluble extraction by supercritical chromatography extraction;
d. the boswellic acid fraction is the remainder of the supercritical chromatography extraction process and may be acetylated to produce an acetylated boswellica acid fraction; and
e. the polysaccharide fraction is derived by hot water extraction of the remainder of the lipid soluble chemical constituent extraction.

36. The method of claim 35, wherein the method for essential extraction comprises:
a. loading in an extraction vessel, an affinity adsorbent and a lipid soluble chemical constituent extract mixture;
b. adding carbon dioxide under supercritical conditions;
c. contacting the feedstock-affinity adsorbent mixture and the carbon dioxide for a time; and
d. collecting an essential oil fraction in a collection vessel.

37. The method of claim 35, wherein a supercritical chromatography carbon dioxide fractional separation system is used for fractionation, purification, and profiling (altering the essential oil chemical constituent compound ratios) of the essential oil fraction.

38. The method of claim 35, wherein the method for boswellic acid fraction extraction comprises optimizing the supercritical chromatography extraction conditions to produce a highly concentrated boswellic acid (triterpenoid) residue extract.

39. The method of claim 35, comprising the additional step of acetylating the purified boswellic acid fraction to produce a highly concentrated acetylated boswellic acid composition wherein about 90% of the non-acetylated boswellic acids have been converted into their acetylated forms.

40. The method of claim 35, wherein the method for polysaccharide fraction extraction comprises:
a. contacting a remainder of a feedstock material from a lipid soluble chemical constituent solvent extraction or ground Boswellia oleo-gum-resin material with a hot water solution for a time sufficient to extract polysaccharide chemical constituent; and
b. separating and purifying the solid polysaccharides from the solution by ethanol precipitation.

41. A method of treating arthritis in a subject comprising administering to a subject in need thereof a therapeutically effective amount of the pharmaceutical composition of claim 34.

42. The method of claim 41, wherein the subject is a primate, bovine, ovine, equine, porcine, rodent, feline, or canine.

43. The method of claim 41, wherein the subject is a human.

44. The method of claim 41, wherein the pharmaceutical composition further comprises a synergistic amount of curcumin.

45. A boswellia species extract comprising a fraction having a Direct Analysis Real Time (DART) mass spectrometry chromatogram of any one of FIG. 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, or 92.

46. The boswellia species extract of claim 45, wherein the extract comprises a compound selected from the group consisting of essential oils, boswellic acids, acetylated boswellic acids, and polysaccharides.

47. The boswellia species extract of claim 46, wherein said compound is selected from the group consisting of the α- and β-boswellic acids and/or their C-acetates comprise alpha-boswellic acid (α-BA), acetyl-alpha-boswellic acid (α-ABA), beta-boswellic acid (β-BA), acetyl-beta-boswellic acid (β-ABA), 9.11-dehydro-alpha-boswellic acid (D-α-BA), acetyl-9.11-dehydro-alpha-boswellic acid (D-α-ABA), 9.11-dehydro-beta-boswellic acid (D-β-BA), acetyl-9.11-dehydro-beta-boswellic acid (D-β-ABA), lupeolic acid (LA), acetyl-lupeolic acid (ALA), 11-keto-beta-boswellic acid (β-KBA), acetyl-11-keto-beta-boswellic acid (β-AKBA), and combinations thereof.

48. The boswellia species of claim 46, wherein the polysaccharide is selected from the group consisting of glucose, arabinose, galactose, rhamnose, xylose uronic acid and combinations thereof.

49. The boswellia species of claim 46, wherein the amount of boswellic acids or acetylated boswellic acids is greater than 65% by weight.

50. The boswellia species extract of claim 46, wherein the amount of essential oil is from 65% to 90% by weight.

51. The boswellia species extract of claim 46, wherein the amount of polysaccharide is greater than 15% by weight.

52. The boswellia species extract of claim 46, wherein the amount of polysaccharide is from 25% to 90% by weight.

53. The boswellia species extract of claim 46, wherein the amount of polysaccharide is from 50% to 90% by weight.

54. The boswellia species extract of claim 46, wherein the amount of polysaccharide is from 75% to 90% by weight.

55. Food or medicament comprising the boswellia species extract of claim 45.
